(19) United States
(12) Patent Application Publication
(10) Pub. No.: US 2024/0226221 A9
(43) Pub. Date: Jul. 11, 2024

(54) CHELIDONIUM MAJUS EXTRACTS AND COMPOSITIONS

(71) Applicant: PHYTHERA THERAPEUTICS INC, San Leandro, CA (US)

(72) Inventors: Igor O. Nasonkin, Irvine, CA (US); Charles Irving, Irvine, CA (US)

(21) Appl. No.: 18/493,383

(22) Filed: Oct. 24, 2023

Related U.S. Application Data
(60) Provisional application No. 63/380,694, filed on Oct. 24, 2022.

(51) Int. Cl. A61K 36/71; A61K 31/4725; A61P 35/00

(52) U.S. Cl. CPC A61K 36/71; A61K 31/4725; A61P 35/00

(57) ABSTRACT

A method of treating a condition comprising: administering a composition, wherein the composition comprises a Chelidonium majus extract and a tariquidar component, and wherein the condition is a cancer related to a gene selected from the group of genes including one or more of: STAG1A, STAG1B, FAM72C, ATF7-NPFF, SLX1B, MTRNR2L10, TMSB15A, Tb15, TbNB, TMSL8, TMSNB, TMSB15, TMSB15B, GP2, CSAG3, DNAJC25-GNG10, PIF1, BIVM, ERCC5, CSNK2A3, CT47A10, DRD2, BRINP2, PNPLA3, KLK13, and/or SERPINA6.

### CROSS-REFERENCE TO RELATED APPLICATIONS

This U.S. Non-Provisional Utility patent application claims the benefit of and priority to U.S. Provisional Patent Application No. 63/380,694 filed on Oct. 24, 2023, by inventor Igor O. Nasonkin the contents of which are expressly incorporated herein by this reference.

### FIELD OF USE

The present disclosure relates generally to methods and compositions related to the Chelidonium genus. More specifically, the present disclosure relates to a using botanical extracts and modifications from botanicals, including Chelidonium majus as a treatment for cancer.

### BACKGROUND

Cancer continues to be a leading cause of morbidity and mortality worldwide, necessitating the exploration of innovative therapeutic strategies. Natural compounds, particularly those derived from plants, have gained substantial attention in recent years due to their potential anti-cancer properties. Certain plant extracts have been identified for their cytotoxic effects on cancer cells, inducing apoptosis and inhibiting the proliferation of malignant cells.

Additionally, modifying agents that enhance the efficacy of these natural compounds in targeting cancer cells are of paramount importance. These modifiers can augment the bioavailability, stability, and specificity of the active components, ensuring their optimal performance in the complex biological milieu of cancerous and/or other tissues.

Accordingly, what is needed is cancer treatment compounds that utilize existing plant extracts that are modified to increase efficacy.

### SUMMARY

To minimize the limitations in the cited references, and to minimize other limitations that will become apparent upon reading and understanding the present specification, the present specification discloses extraction methods and compounds using the extracts to treat cancer.

One embodiment may be a composition comprising: a botanical substance; and a modulator. The botanical substance has antitumor properties. The botanical substance may be a synthesized substance. The botanical substance may be a modified natural substance. The botanical substance may be a Chelidonium majus extract. The modulator may be tariquidar. In other embodiments, the modulator may be tariquidar analogs. The modulator may be a transporter enhancer, such that the modulator causes an increase in uptake into one or more cells of the Chelidonium majus extract. The modulator may be a transporter inhibitor, such that the modulator causes a decrease in outflow from one or more cells of the Chelidonium majus extract. The modulator may be a P-glycoprotein inhibitor, or inhibitor of other cellular functions. The Chelidonium majus extract and tariquidar may be molecularly linked to generate a linked structure.

Another embodiment may be a method of treating a condition comprising the step of: administering a composition; wherein the composition comprises a Chelidonium majus extract and a tariquidar component; and wherein the condition may be a cancer. The cancer may be breast cancer, prostate cancer, lung cancer, gastrointestinal cancer, or skin cancer. The condition may be related to a gene selected from the group consisting of: STAG1A, STAG1B, FAM72C, ATF7-NPFF, SLX1B, MTRNR2L10, TMSB15A, Tb15, TbNB, TMSL8, TMSNB, TMSB15, TMSB15B, GP2, CSAG3, DNAJC25-GNG10, PIF1, BIVM, ERCC5, CSNK2A3, CT47A10, DRD2, BRINP2, PNPLA3, KLK13, and SERPINA6. The administration of the composition may be by application to a user's skin. The administration of the composition may be conducted intravenously. The administration of the composition may be conducted orally. The administration of the composition may be conducted by introducing the composition directly to a tumor. The administration of the composition may be introduced into-cavity (e.g., but not limited to rectal, vaginal, cerebrospinal, ventricular [brain ventricles], ocular etc., and may be administered in gradually dissolving gel-like substance but not limited to that form). The method of treating a condition, wherein nanograms to 100 g (preferably 0.1-10 g) in dry weight of the composition component 1 (botanical extract) and 0.1 mg to 1 g (2-4 mg preferred) of the component 2 (the modulator) per kg of user may be administered per day, or every other day, or weekly, or biweekly. The method of treating a condition, wherein 0.001 micrograms to 100 milligrams (preferably 0.1 to 500 micrograms) in total CLD alkaloid weight of the composition component 1 (botanical extract) and 0.1 mg to 1 g (2-4 mg preferred) of the component 2 (the modulator) per kg of user may be administered per day, or every other day, or weekly, or biweekly. The administration may be once a day, or up to 10 times a day but preferentially 3 times a day, spread more or less evenly in 24 time period. The administration of modulators may be daily, weekly, bi-weekly or other, but preferentially weekly.

One embodiment is the use of the botanical (herbal) drug extracted from the plant belonging to the genus celandine, CLD (Chelidonium majus) and/or the whole genus Chelidonium, or celandine, grown in southern Kazakhstan (around the city of Zharkent) under certain climate conditions, harvested at a certain time of the year, and other similar locations, prepared with a certain protocol from certain parts of the plant, and used for the treatment of all cancer patients with various types of cancers including and especially poorly/not treatable advanced metastatic cancer diseases (metastatic breast cancer, metastatic lung cancer, metastatic melanoma, metastatic brain cancer [neuroblastoma, glioblastoma], metastatic gastro-intestinal & stomach cancers, metastatic prostate cancer, blood cancers, metastatic bone cancer, pancreatic cancers, thyroid cancers, and any other type of tumor or cancer.

Another embodiment may be a special method of preparation of this botanical drug to achieve the maximum treatment efficacy (maximum potency), which includes harvesting of the plant as well as extraction.

Another embodiment may be a special method of administering this botanical drug (treatment regimen) to cancer patients and in general, to any patients, to maximize the treatment efficacy while avoiding exerting toxicity on the body.

Another embodiment may be further modification of this botanical drug with a small molecule, to dramatically enhance this botanical extract's efficacy of killing cancer cells and/or modulating other cellular pathways, which may be therapeutic or affect treatment in some way.

Other features and advantages will become apparent to those skilled in the art from the following detailed description and its accompanying drawings.

### BRIEF DESCRIPTION OF THE DRAWINGS

FIG. 1 shows an outline for an experiment for testing one embodiment of MetPhyte and Tariquidar.

FIG. 2 shows a condition for an experiment for testing one embodiment of MetPhyte and Tariquidar.

FIG. 3 shows the progress of one experiment for testing one embodiment of MetPhyte and Tariquidar after 2 weeks.

FIG. 4 shows the progress of one experiment for testing one embodiment of MetPhyte and Tariquidar after 2 weeks.

FIG. 5 shows the progress of one experiment for testing one embodiment of MetPhyte and Tariquidar after 2 weeks.

FIG. 6 shows the progress of one experiment for testing one embodiment of MetPhyte and Tariquidar after 2 weeks.

FIG. 7 shows the progress of one experiment for testing one embodiment of MetPhyte and Tariquidar after 2 weeks.

FIG. 8 shows the progress of one experiment for testing one embodiment of MetPhyte and Tariquidar after 2 weeks.

FIG. 9 shows the progress of one experiment for testing one embodiment of MetPhyte and Tariquidar after 2 weeks.

FIG. 10 shows the progress of one experiment for testing one embodiment of MetPhyte and Tariquidar after 2 weeks.

FIG. 11 shows the progress of one experiment for testing one embodiment of MetPhyte and Tariquidar after 2.5 weeks.

FIG. 12 shows the progress of one experiment for testing one embodiment of MetPhyte and Tariquidar after 2.5 weeks.

FIG. 13 shows the progress of one experiment for testing one embodiment of MetPhyte and Tariquidar after 2.5 weeks.

FIG. 14 shows the progress of one experiment for testing one embodiment of MetPhyte and Tariquidar after 2.5 weeks.

FIG. 15 shows the progress of one experiment for testing one embodiment of MetPhyte and Tariquidar after 2.5 weeks.

FIG. 16 shows the progress of one experiment for testing one embodiment of MetPhyte and Tariquidar after 2.5 weeks.

FIG. 17 shows the progress of one experiment for testing one embodiment of MetPhyte and Tariquidar after 2.5 weeks.

FIG. 18 shows the progress of one experiment for testing one embodiment of MetPhyte and Tariquidar after 2.5 weeks.

FIG. 19 shows the progress of one experiment for testing one embodiment of MetPhyte and Tariquidar after 2.5 weeks.

FIG. 20 shows the progress of one experiment for testing one embodiment of MetPhyte and Tariquidar after 2.5 weeks.

FIG. 21 shows the progress of one experiment for testing one embodiment of MetPhyte and Tariquidar after 3 weeks.

FIG. 22 shows the progress of one experiment for testing one embodiment of MetPhyte and Tariquidar after 3 weeks.

FIG. 23 shows the progress of one experiment for testing one embodiment of MetPhyte and Tariquidar after 3 weeks.

FIG. 24 shows the progress of one experiment for testing one embodiment of MetPhyte and Tariquidar after 3 weeks.

FIG. 25 shows the progress of one experiment for testing one embodiment of MetPhyte and Tariquidar after 3 weeks.

FIG. 26 shows the progress of one experiment for testing one embodiment of MetPhyte and Tariquidar after 3 weeks.

FIG. 27 shows the progress of one experiment for testing one embodiment of MetPhyte and Tariquidar after 3 weeks.

FIG. 28 shows the progress of one experiment for testing one embodiment of MetPhyte and Tariquidar after 3 weeks.

FIG. 29 shows the progress of one experiment for testing one embodiment of MetPhyte and Tariquidar after 3 weeks.

FIG. 30 shows an outline for an experiment for testing one embodiment of MetPhyte and Tariquidar.

FIG. 31 shows the progress of one experiment for testing one embodiment of MetPhyte and Tariquidar with different amounts of MetPhyte.

FIG. 32 shows the progress of one experiment for testing one embodiment of MetPhyte and Tariquidar with different amounts of MetPhyte.

FIG. 33 shows the progress of one experiment for testing one embodiment of MetPhyte and Tariquidar with different amounts of MetPhyte.

FIG. 34 shows an outline for an experiment for testing one embodiment of MetPhyte and Tariquidar.

FIG. 35 shows the progress of one experiment for testing one embodiment of MetPhyte and Tariquidar with different amounts of MetPhyte.

FIG. 36 shows the progress of one experiment for testing one embodiment of MetPhyte and Tariquidar with different amounts of MetPhyte.

FIG. 37 shows the progress of one experiment for testing one embodiment of MetPhyte and Tariquidar with different amounts of MetPhyte.

FIG. 38 shows an outline for an experiment for testing one embodiment of MetPhyte and Tariquidar.

FIG. 39 shows the progress of one experiment for testing one embodiment of MetPhyte and Tariquidar with different amounts of MetPhyte.

FIG. 40 shows the progress of one experiment for testing one embodiment of MetPhyte and Tariquidar with different amounts of MetPhyte.

FIG. 41 shows the progress of one experiment for testing one embodiment of MetPhyte and Tariquidar with different amounts of MetPhyte.

FIG. 42 shows the progress of one experiment for testing one embodiment of MetPhyte and Tariquidar with different amounts of MetPhyte.

FIG. 43 shows an outline for an experiment for testing one embodiment of MetPhyte and Tariquidar.

FIG. 44 shows the progress of one experiment for testing one embodiment of MetPhyte and Tariquidar with different amounts of MetPhyte.

FIG. 45 shows the progress of one experiment for testing one embodiment of MetPhyte and Tariquidar with different amounts of MetPhyte.

FIG. 46 shows an outline for an experiment for testing one embodiment of MetPhyte and Tariquidar.

FIG. 47 shows the progress of one experiment for testing one embodiment of MetPhyte and Tariquidar with different amounts of MetPhyte.

FIG. 48 shows the progress of one experiment for testing one embodiment of MetPhyte and Tariquidar with different amounts of MetPhyte.

FIG. 49 shows the progress of one experiment for testing one embodiment of MetPhyte and Tariquidar with different amounts of MetPhyte.

FIG. 50 shows an outline for an experiment for testing one embodiment of MetPhyte and Tariquidar.

FIG. 51 shows the progress of one experiment for testing one embodiment of MetPhyte and Tariquidar with different amounts of MetPhyte.

FIG. 52 shows the progress of one experiment for testing one embodiment of MetPhyte and Tariquidar with different amounts of MetPhyte.

FIG. 53 shows the progress of one experiment for testing one embodiment of MetPhyte and Tariquidar with different amounts of MetPhyte.

FIG. 54 shows an outline for an experiment for testing one embodiment of MetPhyte and Tariquidar.

FIG. 55 shows the progress of one experiment for testing one embodiment of MetPhyte and Tariquidar with different amounts of MetPhyte.

FIG. 56 shows the progress of one experiment for testing one embodiment of MetPhyte and Tariquidar with different amounts of MetPhyte.

FIG. 57 shows an outline for an experiment for testing one embodiment of MetPhyte and Tariquidar.

FIG. 58 shows the progress of one experiment for testing one embodiment of MetPhyte and Tariquidar with different amounts of MetPhyte.

FIG. 59 shows the progress of one experiment for testing one embodiment of MetPhyte and Tariquidar with different amounts of MetPhyte.

FIG. 60 shows an outline for an experiment for testing one embodiment of MetPhyte and Tariquidar.

FIG. 61 shows the progress of one experiment for testing one embodiment of MetPhyte and Tariquidar with different amounts of MetPhyte.

FIG. 62 provides an overview of the disclosure of the present application, including motivation to application thereof.

FIG. 63 is a summary of the efficacy of MetPhyte and MetPhyte PLUS on different cell lines.

FIG. 64 shows a summary of the efficacy of MetPhyte.

FIG. 65 is a title page for subsequent figures showing efficacy of MetPhyte on human embryonic carcinoma cells (n2102Ep).

FIG. 66 shows the progress of one experiment for testing one embodiment of MetPhyte on human embryonic carcinoma cells n2102Ep.

FIG. 67 shows the progress of one experiment for testing one embodiment of MetPhyte on human embryonic carcinoma cells n2102Ep.

FIG. 68 shows the progress of one experiment for testing one embodiment of MetPhyte on human embryonic carcinoma cells n2102Ep.

FIG. 69 shows the progress of one experiment for testing one embodiment of MetPhyte on human embryonic carcinoma cells n2102Ep.

FIG. 70 shows the progress of one experiment for testing one embodiment of MetPhyte on human embryonic carcinoma cells n2102Ep.

FIG. 71 shows the progress of one experiment for testing one embodiment of MetPhyte on human embryonic carcinoma cells n2102Ep.

FIG. 72 is a title page for subsequent figures showing efficacy of MetPhyte on human osteosarcoma (CRL1543) and human mammary carcinoma (CRL1543) cells.

FIG. 73 shows the progress of one experiment for testing one embodiment of MetPhyte on human osteosarcoma cells n2102Ep.

FIG. 74 shows the progress of one experiment for testing one embodiment of MetPhyte on human mammary adenocarcinoma cells CRL2351.

FIG. 75 is a title page for subsequent figures showing efficacy of MetPhyte on human primary fibroblast cell and human primary newborn foreskin cell results.

FIG. 76 shows the progress of one experiment for testing one embodiment of MetPhyte on human primary adult fibroblasts.

FIG. 77 shows the progress of one experiment for testing one embodiment of MetPhyte on human primary newborn foreskin fibroblasts.

FIG. 78 is a chart identifying a series of gene related cancers and conditions and potential pathways through which MetPhyte may have an effect.

FIGS. 79-80 are diagrams showing how combining MetPhyte or MetPhyte PLUS, and liposomes or exosomes or any other natural or artificial small vehicles/particles, comprising lipids or/and proteins or/and sugars or/and any other organic or/and inorganic matter [e.g., magnetic particles, dextran particles, any] is expected to work.

FIG. 81 shows MetPhyte without a PLUS component.

FIG. 82 shows MetPlyte PLUS.

FIG. 83 is a diagram showing one embodiment of application via a microneedle patch.

FIG. 84 is a diagram showing one embodiment of application via a microneedle patch.

FIG. 85 is a diagram showing one embodiment of application via intra bone injection.

FIG. 86 is a diagram showing one embodiment of application of MetPhyte Plus on chicken embryos.

### DETAILED DESCRIPTION

The drawings show illustrative embodiments, but do not depict all embodiments. Other embodiments may be used in addition to or instead of the illustrative embodiments. Details that may be apparent or unnecessary may be omitted for the purpose of saving space or for more effective illustrations. Some embodiments may be practiced with additional components or steps and/or without some or all components or steps provided in the illustrations. When different drawings contain the same numeral, that numeral refers to the same or similar components or steps.

In the following detailed description of various embodiments, numerous specific details are set forth in order to provide a thorough understanding of various aspects of the embodiments. However, the embodiments may be practiced without some or all of these specific details. In other instances, well-known procedures and/or components have not been described in detail so as not to unnecessarily obscure aspects of the embodiments.

While some embodiments are disclosed here, other embodiments will become obvious to those skilled in the art as a result of the following detailed description. These embodiments are capable of modifications of various obvious aspects, all without departing from the spirit and scope of protection. The Figures, and their detailed descriptions, are to be regarded as illustrative in nature and not restrictive. Also, the reference or non-reference to a particular embodiment shall not be interpreted to limit the scope of protection.

In the following description, certain terminology is used to describe certain features of one or more embodiments. For purposes of the specification, unless otherwise specified, the term “substantially” refers to the complete or nearly complete extent or degree of an action, characteristic, property, state, structure, item, group of items, or result. For example, in one embodiment, an object that is “substantially” located within a housing would mean that the object is either completely within a housing or nearly completely within a housing. The exact allowable degree of deviation from absolute completeness may in some cases depend on the specific context. However, generally speaking, the nearness of completion will be so as to have the same overall result as if absolute and total completion were obtained. The use of “substantially” is also equally applicable when used in a negative connotation to refer to the complete or near complete lack of an action, characteristic, property, state, structure, item, group of items, or result. In another example, substantially all of a group of items may include all of the items of that group, or at least all of the items of that group that are generally within the normal parameters for the items. To the extent that the group of items might include members that far exceed the normal parameters, this abnormal item might not be expected to be part of substantially all the group of items.

As used herein, the terms “approximately” and “about” generally refer to a deviance of within 5% of the indicated number or range of numbers. In one embodiment, the term “approximately” and “about”, may refer to a deviance of between 0.0001-40% from the indicated number or range of numbers.

Botanical drugs are a special class of drugs described by the US Food and Drug Administration (FDA) and have a well-defined (though continually changing) path to Investigational New Drug (IND) approval by FDA: https://www.fda.gov/files/drugs/published/Botanical-Drug-Development--Guidance-for-Industry.pdf; https://www.fda.gov/about-fda/center-drug-evaluation-and-research-cder/what-botanical-drug.

### Definitions

The term MetPhyte, as used herein, refers to botanical extract from Chelidonium majus (described and prepared as above and administered using a regimen described in this disclosure).

The term MetPhyte PLUS refers to MetPhyte used in combination with an additional small chemical molecule (or molecules). MetPhyte PLUS may include any chemical molecule of the classes, which can modulate the potency of the original botanical preparation, not only a preparation from Chelidonium majus.

Some embodiments may describe a uniquely prepared combination of a botanical drug+a small molecule (substantially enhancing such botanical drug) for treating cancer, including but not limited to advanced (metastatic) cancer and in general for controlling any unwanted biological behavior/biological state of cells (including but not limited to cell proliferation, cell migration, cell death, multipotency, chromosomal segregation, DNA replication, any unwanted state of cell homeostasis etc.), including for cell therapy applications, collectively, making it a novel class of drug (botanical+), with no precedent of formulation/broad use of such combination before for treating cancer or the any unwanted biological behavior proliferation of cells described above in 00100. One embodiment may be referred to as MetPhyte PLUS.

Some embodiments may include an original and unique treatment regimen for cancer patients or any unwanted cell proliferation, which administers botanical drugs (MetPhyte, and MetPhyte PLUS) in a way to achieve the maximum impact of the botanical drug on cancer cells cancer patients or any unwanted biological behavior/biological state of cells (including but not limited to cell proliferation, cell migration, cell death, multipotency, chromosomal segregation, DNA replication, any unwanted state of cell homeostasis etc.

One embodiment may include using a unique treatment regimen (of patients with metastatic cancer or any unwanted cell proliferation in vivo or in cultured cells or in ovo) using such concentrated CLD botanical extract (described hereinbelow), which may enable the body to sustain the toxic impact of the drug, become adjusted to it, while causing cancer cells, including but not limited to metastatic cancer, inside the body or any unwanted biological behavior/biological state of cells (described above), including but not limited to cell proliferation (in vivo, cultured or in ovo) to stop dividing, stop metastatic growth and spreading, and stop killing a patient or to stop unwanted proliferation, collectively causing either cancer regression or complete healing or stop any negative impacts associated with unwanted cell proliferation (in vivo, cultured, or in ovo).

One embodiment may be a novel method of preparation of such botanical extract, which involves harvesting the plant at a certain time of the year, using certain parts of the plant, and using a certain concentration of the drug in a certain extraction medium, all aimed to achieve the highest concentration of bioactive small molecules (expected: mostly but not exclusive to alkaloids) in such herbal extract (collectively, highest potency to kill cancer cells or the definition: any unwanted proliferation of cells in vivo, cultured cells, or in ovo).

In one embodiment, MetPhyte may be a concentrated botanical extract of CLD (between 0.1 kilogram and 10 kilograms of raw, just harvested, plant per 1 liter of 40% volume per volume [range 0.1% to 99.9% but most typically 40%] ethyl alcohol and 60% [range, volume: volume 99.9% to 0.1% but most typically 60%] water [preferably rich in salts, including but not limited to salts of Silver, Argentum, Ag or any inorganic molecules].

### Benefits

One benefit may be efficacy of treating cancer (any cancer), especially but not limited to advanced metastatic cancers (3-4 stage), and especially those (but not limited to) cancer patients, whose cancer is resistant to chemotherapy and/or is hard/impossible to be treated with surgery (when metastases spread beyond the primary site and impossible/hard to resect surgically; this is especially relevant to brain cancer cases, when tumor masses are surrounded by vasculature and are seeded at many locations in normal brain areas: resection becomes impossible in such cases). And, efficacy of treating any over-proliferation of cells, including in vivo, cultured cells, and in ovo.

Another benefit may be reducing the dosage of chemotherapy and by doing so, reducing the overall cost of treatment (if the MetPhyte or MetPhyte PLUS are used in combination with chemotherapy).

00107 Another benefit may be reducing the toxicity of ligands or/and modulating the biological action of compounds working together with chemotherapy treatments, such as but not limited to Dopamine receptors ligands https://www.ncbi.nlm.nih.gov/pmc/articles/PMC8255587/

Another benefit may be substantially increasing the quality of life of patients receiving MetPhyte or MetPhyte PLUS treatment, compared to those treated with chemotherapy or/and radiation therapy.

Another benefit may be in some cases, avoiding chemotherapy, radiation therapy or/and surgical resection, altogether, by administering (throughout life, for the rest of patient's life, as courses of treatment, with some 1-6 months interruptions) high but sub-toxic dose of MetPhyte or MetPhyte PLUS to patients with cancer, especially metastatic cancer.

MetPhyte, and especially MetPhyte PLUS, may be well positioned to be the sole treatment (drug) of choice of metastatic cancer patients (stage 3 and especially 4) whose previous treatments failed twice or more (e.g., patients who were not cured by 2 or more different types of chemo or radiation).

One of the conceptualized methods of MetPhyte, and MetPhyte PLUS delivery to cancer patients (broadly speaking, patients suffering from cell over-proliferation) is targeted delivery of MetPhyte and MetPhyte PLUS directly to the tumor.

Another benefit may be ease of use/simplicity in administering this drug to patients (patients can take this medicine at home guided by physician's instruction, easy, painless, no need to be in a hospital=cheap, no need for transportation, collectively reducing patient's burden, from financial to emotional to logistics, associated with treatment). The currently used treatment regimen with MetPhyte is to drink this medication with water between 2-3 times a day, in time intervals spaced approximately evenly within each 24 hours. This is compared to intravenous infusion of toxic chemotherapy drugs in a hospital (cost, unpleasant, toxic, logistical, financial and emotional burden).

Another benefit may be the economy in manufacturing (which translates into a reduced cost to patients and healthcare insurance): this botanical drug is currently extracted from a Chelidonium plant by grinding the plant and extracting the small molecules such as but not limited to alkaloids terpenoids, flavonoids, lectins using the 40% ethyl alcohol solution (described above) or oil-based solution (such as but not limited to olive or grape seed oil, preferentially but not limited to virgin and extra-virgin oil). Even when the costs for growing and production of a plant according to Good Agricultural and Collection Practices (GACP), it is still much less costly to make compared to chemotherapy drugs, which should translate into substantially lowering the overall cost of treatment with MetPhyte and MetPhyte PLUS. https://www.ncbi.nlm.nih.gov/pmc/articles/PMC3538397/; https://ascopubs.org/doi/full/10.1200/EDBK_100028; https://www.nature.com/articles/d41586-018-02483-3.

Another benefit may be the ability to deliver MetPhyte and MetPhyte PLUS in liposomes or exosomes or any other natural or artificial small vehicles/particles, comprising lipids or/and proteins or/and sugars or/and any other organic or/and inorganic matter [e.g., magnetic particles, dextran particles, any], to minimize the toxicity, reduce the concentration of the drugs in the body (reducing the toxicity) while achieving targeted delivery of MetPhyte or MetPhyte PLUS to the tumor cells and thus, improving the efficacy of the drugs (MetPhyte or MetPhyte PLUS) to treat cancer.

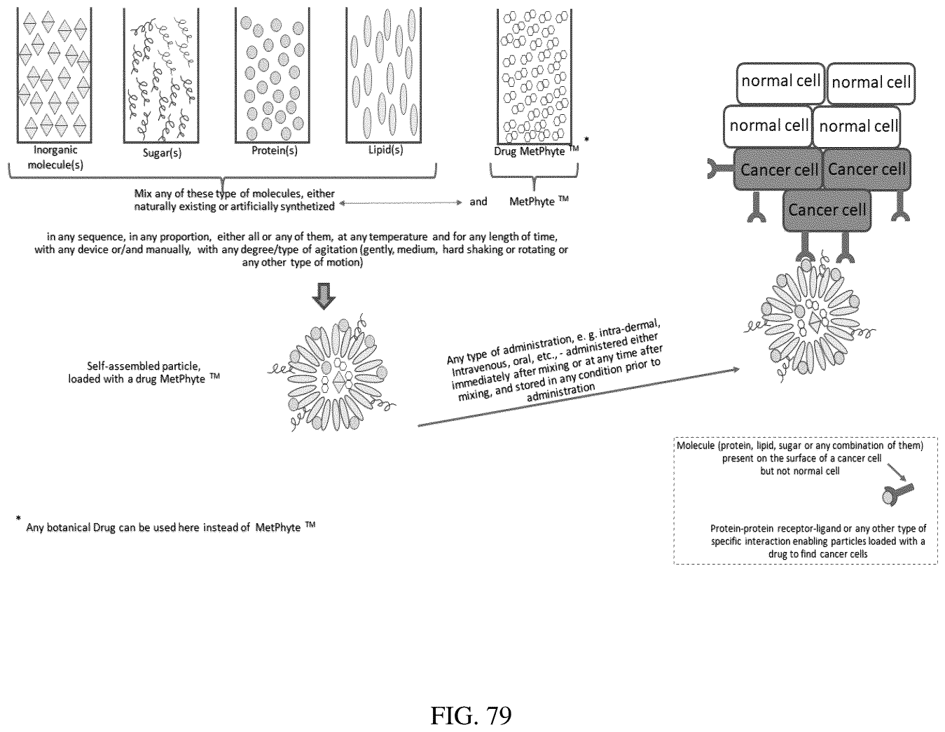

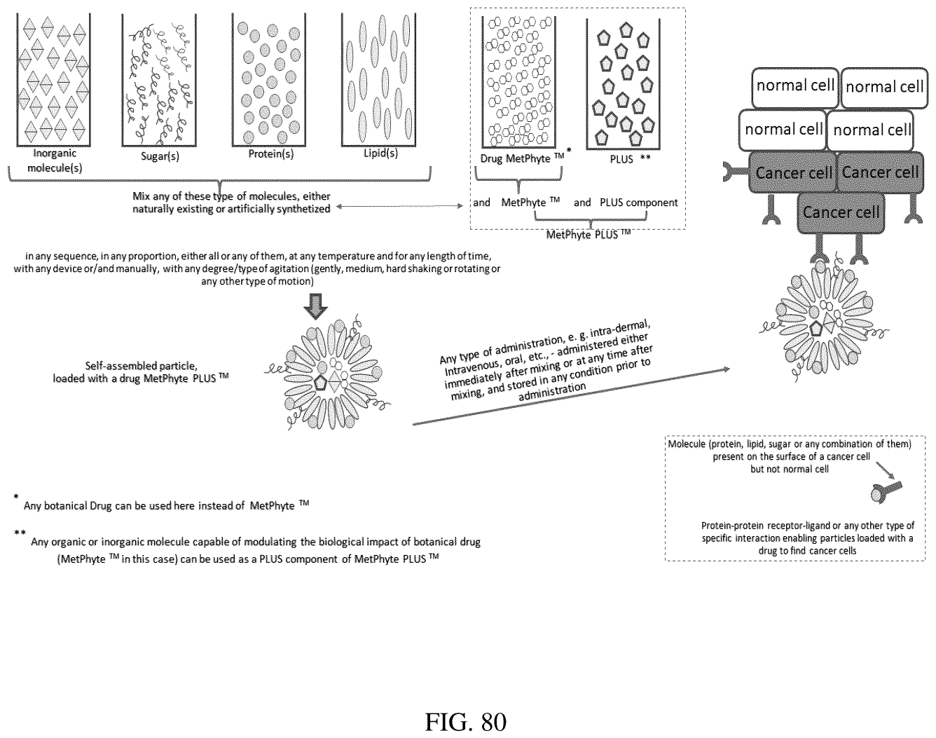

FIGS. 79-80A-B are diagrams showing how combining MetPhyte or MetPhyte PLUS, and liposomes or exosomes or any other natural or artificial small vehicles/particles, comprising lipids or/and proteins or/and sugars or/and any other organic or/and inorganic matter [e.g., magnetic particles, dextran particles, any] is expected to work. This methodology of encapsulation or any other type of interaction of botanical or [botanical PLUS] drug with liposomes or exosomes or any other natural or artificial small vehicles/particles, consisting of lipids or/and proteins or/and sugars or/and any other organic or/and inorganic matter [e.g., magnetic particles, dextran particles, any and/or ALL botanicals, also [Botanical+] drugs, not just MetPhyte or MetPhyte PLUS.

FIG. 79 shows a strategy for targeted delivery of botanical drug (using MetPhyte as an example) specifically to cancer cells but not normal cells.

FIG. 80 shows a strategy for targeted delivery of [botanical+] drug (using MetPhyte as an example) specifically to cancer cells but not normal cells. Any botanical may be used with a PLUS component. PLUS component (in this case MetPhyte with MDR (multi-drug resistant) inhibitor Tariquidar) accumulates inside the cancer cell due to inability of a cell to pump this drug out into the extracellular space. In some cases, the PLUS component is Tariquidar, but it also may be ELACRIDAR, also ZOSUQUIDAR, or a number of other molecules, modulating the drug resistance of cancer cells to drugs, the modulation of multidrug resistance is done by blocking the extrusion of a drug into the extracellular space). Or, the PLUS component may be any of the molecules modulating ABCG2 protein https://pubmed.ncbi.nlm.nih.gov/22509477/ (which contributes to cancer drug resistance phenomenon) or (any type of drug: lipid or protein or small molecule chemical drug or RNA-type of drug [micro-RNA, long-noncoding RNA, mRNA, antisense-RNA, any RNA, collectively]).

In one embodiment, the parts of the methods and compositions may be interrelated as follows:

Part one: the plant belonging to the genus celandine, CLD (in our case Chelidonium majus, but may be any plant in the genus Chelidonium or celandine.

Part two. The plant is preferably grown in southern Kazakhstan (around the city of Zharkent) under certain climate conditions, harvested at a certain time of the year (mid-May to mid-June), prepared with a certain protocol (the plant without or with roots, without or with buds/flowers, but most frequently the plant without roots and with budding shoots and budding flowers as typical for the May-June season—collected, dried or immediately ground (e.g., but not limited to, grinding in a mechanical or electric meatgrinder, which speaks about the extent of grinding) most frequently ground raw, not after drying).

Part three. Extraction: the ground CLD plant material may be (immediately or soon after grinding) mixed with a room-temperature extraction solution, which is the organic solvent, typically: Alcohol-based, which is typically but not limited to:
40% volume/volume medical-grade absolute ethanol (ethyl alcohol) (range between 0.1% and 99.9% volume: volume, but most typically 40%) and
60% volume: volume water (which may be distilled water or deionized water or maybe a local mountain spring water, most typically mountain spring water rich in minerals and salts, including but not limited to silver salts, Ag [Argentum)—let's broaden the types of water and emphasize salts of metals, especially Ag, Argentum) (volume ranges from 99.9% to 0.1%, but most typically 60%) Or sometimes oil-based solvent, which is typically vegetable oil, such as sunflower (but not limited to) oil.
   The temperature of the extraction solvent may be between −20 degrees Celsius (cold extraction) to 100 degrees Celsius (hot extraction), but most frequently room temperature, which is about 25 degrees Celsius. See https://www.ncbi.nlm.nih.gov/pmc/articles/PMC5471285/. After mixing the ground CLD plant and the solvent, the mixture is allowed to stand (extraction) or is periodically rotated or shaken (mechanically or using any type of rotating device) to improve extraction, at temperatures ranging from −20 degrees Celsius to +100 degrees Celsius, most typically room temperature or +4 Celsius), most typically in the dark (limited light or no light) for the time ranging from 1 second to 3 years, typically a month, followed by filtration through a coarse sieve (pores diameter 0.1 millimeters, mm, to 1 centimeter, cm, typically 0.25 cm).

Administration of MetPhyte. Cancer Patients start from low dose (one drop of MetPhyte), which corresponds to approximately 50 microliters of MetPhyte into 100 milliliters of drinking water per dose, taken as a drink immediately after mixing MetPhyte and water (drop to microliter conversion: https://www.unitconverters.net/volume/drop-to-microliter.htm, ranging from 1 microliter to 100 microliters, but most typically 50 microliters) and gradually progress to taking up to 60 drops of MetPhyte/100 ml drinking water per dose, thus, adapting the body to the increased level of MetPhyte patients may self-administer this drug. Patients may take between one to three doses/day in the following way (no interruption for Saturdays or Sundays or any other days.

TABLE I

| Consecutive days | morning | noon | evening | Total/day |
|---|---|---|---|---|
| Day1 = 1^{st} day | 1 drop | 1 drop | 1 drop | 3 drops |
| Day2 | 2 drops | 2 drops | 2 drops | 6 drops |
| Day3 | 3 drops | 3 drops | 3 drops | 9 drops |
| Day4 | 4 drops | 4 drops | 4 drops | 12 drops |
| Day5 | 5 drops | 5 drops | 5 drops | 15 drops |
| Day6 | 6 drops | 6 drops | 6 drops | 18 drops |
| Day7 | 7 drops | 7 drops | 7 drops | 21 drops |
| Day8 | 8 drops | 8 drops | 8 drops | 24 drops |
| Day9 | 9 drops | 9 drops | 9 drops | 27 drops |
| Day10 | 10 drops | 10 drops | 10 drops | 30 drops |
| Day11 | 11 drops | 11 drops | 11 drops | 33 drops |
| Day12 | 12 drops | 12 drops | 12 drops | 36 drops |
| Day13 | 13 drops | 13 drops | 13 drops | 39 drops |
| Day14 | 14 drops | 14 drops | 14 drops | 42 drops |
| Day15 | 15 drops | 15 drops | 15 drops | 45 drops |
| Day16 | 16 drops | 16 drops | 16 drops | 48 drops |
| Day17 | 17 drops | 17 drops | 17 drops | 51 drops |
| Day18 | 18 drops | 18 drops | 18 drops | 54 drops |
| Day19 | 19 drops | 19 drops | 19 drops | 57 drops |
| Day20 | 20 drops | 20 drops | 20 drops | 60 drops |
| Day21 | 21 drops | 21 drops | 21 drops | 63 drops |

In the embodiment described here, the drop is approximately 50 microliters. The concentration of small molecules in CLD extract is evaluated as measure of total alkaloids, which could be is in the range of 0.01-10 micrograms per microliter of extract but most typically around 0.1-0.5 micrograms per microliter of extract.

Note: the concentration of the CLD extract could be adjusted to a certain desired concentration in the range shown, to standardize treatment and compare data between patients.
If a patient is taking one dose/day, then it is:
Starting: 1 drop . . . day 1
Ending: 21 drops . . . day 21
   If a patient is taking 2 drops/day, then it is:
Starting: 2 drops . . . day 1 (1 drop in the morning and 1 drop in the evening)
Ending: 42 drops . . . day 21 (2 drops in the morning and 2 drops in the evening)

In one embodiment, cancer patients may be observed periodically by a physician, as each patient depending on body size/weight (BMI, body mass index) may start experiencing an overdose (signs of toxicity) sooner or later, with a major sign being tremors of fingers and hands. Then a patient should stop increasing the dosage (e.g., at drop 10 or drop14). As one alternative, a patient then stops taking MetPhyte for a period of one to 60 days, most commonly 2 weeks; then repeats the regimen and stops at the dosage, which previously caused tremor (threshold limit of MetPhyte/body size). Then repeat again.

As another alternative, the patients continue taking MetPhyte without interruption, but by gradually decreasing the daily dosage of MetPhyte, the following way:
First, increasing from 1 drop to up to 41 drops, but preferentially up to 21 drop/dose
After highest tolerable dose is reached (determined in some cases by mild tremor of hands [limit of toxicity reached]—decreasing from 21 drop/dose to 1 drop/dose

TABLE II

| Consecutive days | morning | noon | evening | Total/day |
|---|---|---|---|---|
| Day1 = 1^{st} day | 1 drop | 1 drop | 1 drop | 3 drops |
| Day2 | 2 drops | 2 drops | 2 drops | 6 drops |
| Day3 | 3 drops | 3 drops | 3 drops | 9 drops |
| Day4 | 4 drops | 4 drops | 4 drops | 12 drops |
| Day5 | 5 drops | 5 drops | 5 drops | 15 drops |
| Day6 | 6 drops | 6 drops | 6 drops | 18 drops |
| Day7 | 7 drops | 7 drops | 7 drops | 21 drops |
| Day8 | 8 drops | 8 drops | 8 drops | 24 drops |
| Day9 | 9 drops | 9 drops | 9 drops | 27 drops |
| Day10 | 10 drops | 10 drops | 10 drops | 30 drops |
| Day11 | 11 drops | 11 drops | 11 drops | 33 drops |
| Day12 | 12 drops | 12 drops | 12 drops | 36 drops |
| Day13 | 13 drops | 13 drops | 13 drops | 39 drops |
| Day14 | 14 drops | 14 drops | 14 drops | 42 drops |
| Day15 | 15 drops | 15 drops | 15 drops | 45 drops |
| Day16 | 16 drops | 16 drops | 16 drops | 48 drops |
| Day17 | 17 drops | 17 drops | 17 drops | 51 drops |
| Day18 | 18 drops | 18 drops | 18 drops | 54 drops |
| Day19 | 19 drops | 19 drops | 19 drops | 57 drops |
| Day20 | 20 drops | 20 drops | 20 drops | 60 drops |
| Day21 | 21 drops | 21 drops | 21 drops | 63 drops |
| Day22 | 21 drops | 21 drops | 21 drops | 63 drops |
| Day23 | 20 drops | 20 drops | 20 drops | 60 drops |
| Day24 | 19 drops | 19 drops | 19 drops | 57 drops |
| Day25 | 18 drops | 18 drops | 18 drops | 54 drops |
| Day26 | 17 drops | 17 drops | 17 drops | 51 drops |
| Day27 | 16 drops | 16 drops | 16 drops | 48 drops |
| Day28 | 15 drops | 15 drops | 15 drops | 45 drops |
| Day29 | 14 drops | 14 drops | 14 drops | 42 drops |
| Day30 | 13 drops | 13 drops | 13 drops | 39 drops |
| Day31 | 12 drops | 12 drops | 12 drops | 36 drops |
| Day32 | 11 drops | 11 drops | 11 drops | 33 drops |
| Day33 | 10 drops | 10 drops | 10 drops | 30 drops |
| Day34 | 9 drops | 9 drops | 9 drops | 27 drops |
| Day35 | 8 drops | 8 drops | 8 drops | 24 drops |
| Day36 | 7 drops | 7 drops | 7 drops | 21 drops |
| Day37 | 6 drops | 6 drops | 6 drops | 18 drops |
| Day38 | 5 drops | 5 drops | 5 drops | 15 drops |
| Day39 | 4 drops | 4 drops | 4 drops | 12 drops |
| Day40 | 3 drops | 3 drops | 3 drops | 9 drops |
| Day41 | 2 drops | 2 drops | 2 drops | 6 drops |
| Day42 | 1 drop | 1 drop | 1 drop | 3 drops |

In most cancer patients the threshold is reached at the dosage of 60 drops/day or less but in some cases, such as advanced metastatic cancer, up to 41 drops at a time may be needed (resulting in patient taking 41 drops in the morning, 41 drops in the day time, and 41 drops in the evening).
For more advanced, aggressive cancer cases (e.g., stage 4 with multiple metastases) it is plausible to administer the maximum MetPhyte dosage/day, which is higher than 63 drops/day (e.g., continue with day 22: 22, 22, 22, etc., drops; day23: 23, 23, 23 drops, etc., until signs of toxicity will be not manageable (tremors of extremities) This treatment preferably continued throughout the lifetime of a patient with metastatic cancer, to prevent/reduce new metastases. Yet another treatment regimen, for more aggressive cancer cases, is increasing the dosage of MetPhyte more quickly/day, such as:

TABLE III

| Consecutive days | morning | noon | evening | Total/day |
|---|---|---|---|---|
| Day1 = 1^{st} day | 1 drop | 2 drops | 3 drops | 6 drops |
| Day2 | 4 drops | 5 drops | 6 drops | 15 drops |
| Day3 | 7 drops | 8 drops | 9 drops | 24 drops |
| Day4 | 10 drops | 11 drops | 12 drops | 33 drops |
| Day5 | 13 drops | 14 drops | 15 drops | 42 drops |
| Day6 | 16 drops | 17 drops | 18 drops | 51 drops |
| Day7 | 19 drops | 20 drops | 21 drops | 60 drops |
| Day8 | 22 drops | 23 drops | 24 drops | 69 drops |
| Day9 | 25 drops | 26 drops | 27 drops | 78 drops |
| Day10 | 28 drops | 29 drops | 30 drops | 87 drops |

In most patients the highest tolerated dose of MetPhyte (before the overdose symptoms kick in: tremor of the extremities as most obvious sign) is 21 drops (day 7), though in some cases the maximum tolerated dosage may be higher, such as in heavy or/and tall people (i.e., to reach the toxic levels of MetPhyte they require higher dosage of MetPhyte).

As one alternative, a patient then stops taking MetPhyte for a period of one to 60 days, most commonly 2 weeks; then repeats the regimen and stops at the dosage, which previously caused tremor (threshold limit of MetPhyte/body size). Then repeat again.

As another alternative, the patients continue taking MetPhyte without interruption, but by gradually decreasing the daily dosage of MetPhyte, the following way:
xxxxxxxxxX—increasing from 1 drop to 30 drop/dose
Xxxxxxxxxx—decreasing from 30 drop/dose to 1 drop/dose

TABLE IV

| Consecutive days | morning | noon | evening | Total/day |
|---|---|---|---|---|
| Day1 = 1^{st} day | 1 drop | 2 drops | 3 drops | 6 drops |
| Day2 | 4 drops | 5 drops | 6 drops | 15 drops |
| Day3 | 7 drops | 8 drops | 9 drops | 24 drops |
| Day4 | 10 drops | 11 drops | 12 drops | 33 drops |
| Day5 | 13 drops | 14 drops | 15 drops | 42 drops |
| Day6 | 16 drops | 17 drops | 18 drops | 51 drops |
| Day7 | 19 drops | 20 drops | 21 drops | 60 drops |
| Day8 | 22 drops | 23 drops | 24 drops | 69 drops |
| Day9 | 25 drops | 26 drops | 27 drops | 78 drops |
| Day10 | 28 drops | 29 drops | 30 drops | 87 drops |
| Day11 | 30 drops | 29 drops | 28 drops | 87 drops |
| Day12 | 27 drops | 26 drops | 25 drops | 78 drops |
| Day13 | 24 drops | 23 drops | 22 drops | 69 drops |
| Day14 | 21 drops | 20 drops | 19 drops | 60 drops |
| Day15 | 18 drops | 17 drops | 16 drops | 51 drops |
| Day16 | 15 drops | 14 drops | 13 drops | 42 drops |
| Day17 | 12 drops | 11 drops | 10 drops | 33 drops |
| Day18 | 9 drops | 8 drops | 7 drops | 24 drops |
| Day19 | 6 drops | 5 drops | 4 drops | 15 drops |
| Day20 | 3 drops | 2 drops | 1 drop | 6 drops |

Most patients may tolerate well the dose of up to 21 drops/dose before experiencing side effects (tremors of the extremities). Then one should gradually decrease the dosage as above, e.g., day 8: 21 drops; 20 drops; 19 Day14: 3 drops; 2 drops; 1 drop.

When tested and validated in cultured human cancer cells, the following data were obtained for MetPhyte (stock: 2021 annual harvest & preparation; and stock: 2022 annual harvest and preparation).

### The Cell Lines:

TABLE V

| | | | Cells Killed |
|---|---|---|---|
| | | Cells Killed | MetPhyte ™ |
| Cancer Type | Cell line, source | MetPhyte ™ | PLUS |
| Teratocarcinoma | N2102Ep, UK | ✓ | |
| Mammary | CRL2351 AU56S | ✓ | ✓✓✓ |
| adenocarcinoma | ATCC | | |
| Osteosarcoma | CRL1543 ATCC | ✓ | ✓✓✓ |
| Melanoma | SK-MEL-2 HTB- | ✓ | |
| | 68 ATCC | | |
| Melanoma | SK-MEL-1 Addex Bio | ✓ | ✓✓ |
| Melanoma | A375 Addex Bio | ✓ | ✓✓ |
| Neuroblastoma | CRL2149 SK-N- | X | ✓✓ |
| | DZ ATCC | | |
| Retinoblastoma | Y-79 Addex Bio | X | ✓ |
| Foreskin, | Foreskin Fibroblasts | X | X |
| primary | XGene | | |
| Adult fibroblasts, | PC-1 Adult fibroblasts | X | X |
| primary | BioTime | | |

Both preparations (MetPhyte and MetPhyte PLUS were able to kill human cancer cells (but not normal human fibroblasts=controls). For MetPhyte, the dose of this drug capable of killing a lawn of human cancer cells was between 50-200 microliters of MetPhyte/10 ml tissue culture media (normally around 150 microliters of MetPhyte), when the drug was added one or twice a week (every 3-7 days). For MetPhyte PLUS a 50% lethal dose (LD50) was in the range of 10-50 microliters of MetPhyte/10 ml tissue culture media, typically 20 microliters of MetPhyte/10 milliliters (10,000 microliters) of tissue culture media, when a PLUS component was added once or twice a week and the drug MetPhyte was added one or twice a week (every 3-7 days).

If converted to dry weight of MetPhyte: from nanogram to 100 grams of dried/lyophilized preparation/per 1 million cells (or, per 1 kg of tissue in the body), but preferably 0.1-10 mg of botanical drug (dry weight)/1 million cells (or 0.1-10 grams of botanical drug (dry weight)/1 kg body weight). For Tariquidar (PLUS component) or any other molecule, modulating the activity of botanical drug: (from 0.001 to 100 microliters, but preferentially, 1-10 microliters of 10 milli Molar Tariquidar/10 ml tissue culture media)—added with MetPhyte, every day or every other day or every 3rd day or every 7th day, when testing on cancer cells. Which is 1-10 micromolar concentration of Tariquidar in tissue culture media. Or. From 0.1 milligram to 1 grams Tariquidar (PLUS component)/1 kilogram of body weight, (or any other molecule, other than Tariquidar, modulating the activity of botanical drug), but preferably around 2 mg Tariquidar/1 kg body weight.

As described above, the MetPhyte is prepared from Chelidonium majus (the greater celandine) or from any plant in the whole genus Chelidonium, or celandine. The alkaloids and potentially other small molecules in botanical extract from Chelidonium (e.g., but not limited to terpenoids, lectins and others) are expected to impact cell division, induce cell death of the rapidly dividing cancer cells via apoptosis, potentially modulate the epigenome (DNA methylation and histone methylation-acetylation-other histone modifications on chromatin), also modulate epithelial-to-mesenchymal transition (EMT) (which modulates the migratory properties of metastatic cancer cells, or their “invasiveness”), collectively modulate any unwanted biological behavior/biological state of cells (including but not limited to cell proliferation, cell migration, cell death, multipotency, chromosomal segregation, DNA replication, any unwanted state of cell homeostasis etc.). The modulation of the EMT activity may also include such properties of the cancer cell as “stickiness (the strength of cell-to-cell adhesion and collectively, the adhesion of individual cells in cancer mass to the whole mass of cancer cells; the less adhesion/stickiness—the more aggressive is metastatic activity; MetPhyte is expected to modulate such adhesion (preferentially reduce it, but not limited to reduction).

The minerals and salts contained in the water component of this extract (e.g., but not limited to Argentum, AG, silver) may also modulate cancer cell metabolism and induce cell death via variable mechanisms such as, but not limited to apoptosis, for example, but not limited to, as serving co-factors for the enzymatic or ligand-based biological processes, modulated by the drugs. Collectively, the actions of these molecules, organic and inorganic, in MetPhyte may cause cancer cells to:

Reduce metastatic activity (reduce invasiveness);
Decrease/stop cell division=self-renewal (this includes modulation of cell division machinery of the cancer cells—any pathway; also, modulation of the epigenome (which includes DNA methylation and histone modifications);
Initiate cell death, preferentially via apoptosis (apoptotic mechanism), but not limited to apoptosis;
Potentially decrease the number of circulating tumor cells (CTCs), same as tumor-initiating cells (TICs), which are present in the blood of patients with metastatic cancers and seed/initiate 2ndary, tertiary etc. site of tumor growth (metastases). This can be done via modulation of the “sternness” property of cancer cells including CTCs, or TICs (stemness includes epigenetic state, cell renewal properties, differentiation properties) and induction of maturation (acquisition of more mature cell states from immature/poorly differentiated, which CTCs, or TICs are). This, in turn, causes aggressive metastases (sites of dividing cancer cells, disseminating aggressive cancer cells to other sites in the body) to stop dividing, encapsulate, and become dormant, rather than aggressive and metastatic).
Change/modulate cellular metabolism to make it more typical for a normal cell and less typical for cancer cell, thus slowing down/halting formation of new metastases, reducing the number of CTCs or TICs, reducing cell migration and invasiveness (contributing to metastatic properties).
Collectively, modulate any unwanted biological behavior/biological state of cells (including but not limited to cell proliferation, cell migration, cell death, multipotency, chromosomal segregation, DNA replication, any unwanted state of cell homeostasis etc.).

How the MetPhyte PLUS works:

In this embodiment the botanical component of MetPhyte is described above, and the PLUS component of MetPhyte PLUS is Tariquidar.
https://www.sigmaaldrich.com/US/en/product/sigma/sml1790
http://www.apexbt.com/tariquidar.html
https://www.caymanchem.com/product/24180/tariquidar

However, it may be a molecule from the same class of molecules as Tariquidar (P-glycoprotein inhibitor, same as Multidrug resistance protein inhibitor—any of the several Multidrug resistance protein inhibitors). For example, ELACRIDAR, also ZOSUQUIDAR. Or, it can be any other organic or inorganic molecule (small or large), which can modulate (preferentially/desirably, inhibit, but not limited to inhibition) the activity of P-glycoprotein, same as P-gp., same as Multidrug resistance protein (or, any of the several Multidrug resistance proteins, in our case MDR1, the same as ABCB1). Or, it can be any other organic or inorganic molecule (small or large), which can modulate (preferentially/desirably inhibit, but not limited to inhibition) the protein, called ABCG2: ABCG2 is a constitutively expressed ATP-binding cassette (ABC) transporter that protects many tissues against xenobiotic molecules. Its activity affects the pharmacokinetics of commonly used drugs and limits the delivery of therapeutics into tumor cells, thus contributing to multidrug resistance. https://www.nature.com/articles/nature22345.

Or efficacy may be related to a molecule/molecules, which modulate any other cell proteins or any other molecules, controlling other transporters (membrane or mitochondrial) or any other ligand-receptor interactions, or other biological pathways, which modulate the interaction of CLD small molecules with cellular biological pathways, collectively resulting in the enhanced impact of the CLD small molecules on cells, causing the desired therapeutic or other outcomes.

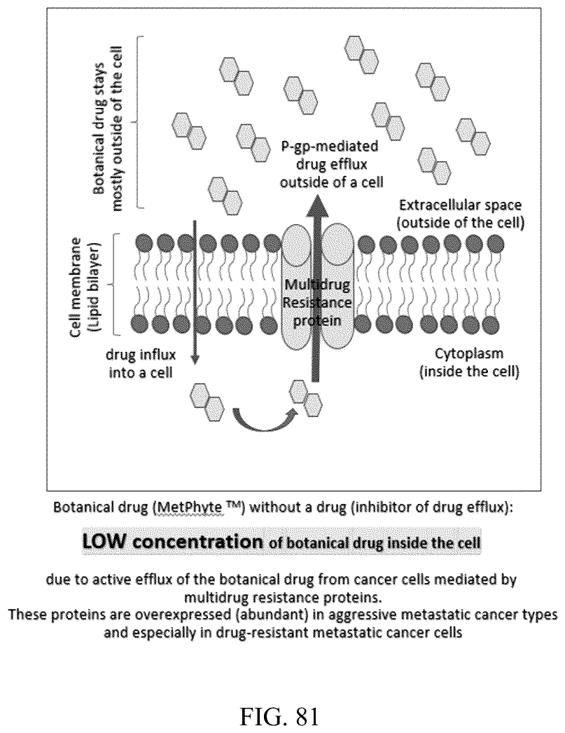

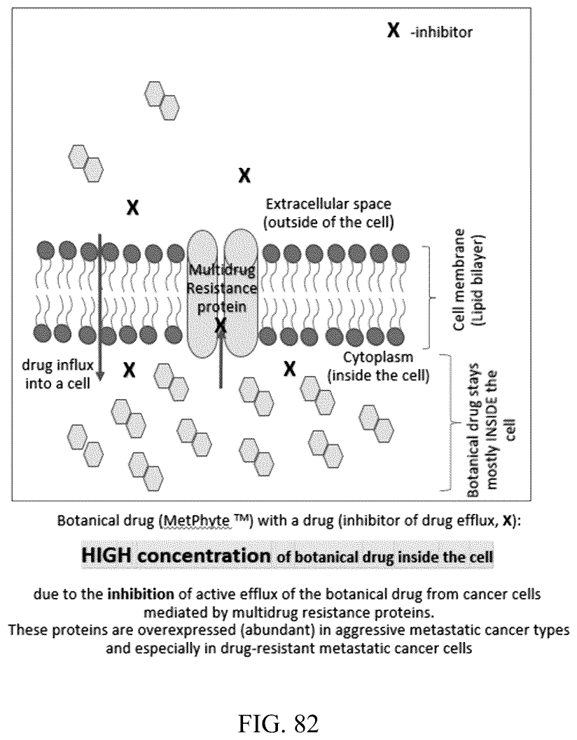

Specifically, in relation to Tariquidar (also ELACRIDAR, also ZOSUQUIDAR). Or a number of other molecules, modulating the drug resistance of cancer cells to drugs, the modulation of multidrug resistance is done by blocking the extrusion of a drug into the extracellular space, thus creating a higher concentration of a drug in the cancer cells, and thus enabling using lower concentration of a drug to treat cancer, and lowering the toxicity of a drug to the rest of the body this is illustrated in FIGS. 81-82 below.

FIG. 81 shows MetPhyte without a PLUS component (or here, the PLUS component is Tariquidar, but it also may be ELACRIDAR, also ZOSUQUIDAR) or various other molecules, modulating the drug resistance of cancer cells to drugs, the modulation of multidrug resistance is done by blocking the extrusion of a drug into the extracellular space). Or, the PLUS component can be any of the molecules modulating ABCG2 protein https://pubmed.ncbi.nlm.nih.gov/22509477/ (which contributes to cancer drug resistance phenomenon), (any type of drug: lipid or protein or small molecule chemical drug or RNA-type of drug [micro-RNA, long-noncoding RNA, mRNA, antisense-RNA, any RNA, collectively]).

FIG. 82 shows MetPlyte PLUS (in this case MetPhyte with MDR inhibitor Tariquidar) accumulates inside the cancer cell due to inability of a cell to pump this drug out into the extracellular space. In our case the PLUS component is Tariquidar, but it also may be ELACRIDAR, also ZOSUQUIDAR or a number of other molecules, modulating the drug resistance of cancer cells to drugs, the modulation of multidrug resistance is done by blocking the extrusion of a drug into the extracellular space. Or the PLUS component can be any of the molecules modulating ABCG2 protein https://pubmed.ncbi.nlm.nih.gov/22509477/(which contributes to cancer drug resistance phenomenon). Any type of drug: lipid or protein or small molecule chemical drug or RNA-type of drug [micro-RNA, long-noncoding RNA, mRNA, antisense-RNA, any RNA, collectively] may be used.

In various embodiments, the present disclosure may cover any botanical drug and any PLUS drug.

Delivery method alternatives:
oral administration via drops [in a glass of water or any liquid] or gelatinized or lyophilized pills with admixture of any suitable substance as a filler/carrier (Pharmaceutical excipients) https://www.drugtopics.com/view/overview-pharmaceutical-excipients-used-tablets-and-capsules
rectal or vaginal administration via syringe or any other device to introduce a drug via rectum (MetPhyte or MetPhyte PLUS mixed with any liquid, e.g., HBSS, or other solution used for intravenous injection of drugs] or gelatinized or lyophilized pills with admixture of any suitable substance as a filler/carrier (Pharmaceutical excipients) https://www.drugtopics.com/view/overview-pharmaceutical-excipients-used-tablets-and-capsules
intravenous administration (of MetPhyte or MetPhyte PLUS mixed with water or any liquid)
intra-cranial administration (which includes intra-ventricular administration) of MetPhyte or MetPhyte PLUS mixed with any liquid, e.g., HBSS, or other solution used for intravenous or intra-cranial injection of drugs;
intrathecal administration (via injection into the spinal canal or into subarachnoid space, collectively, to enable the drug to reach cerebrospinal fluid https://en.wikipedia.org/wiki/Intrathecal_administration;
Incorporation of MetPhyte™ or MetPhyte™ PLUS into any type of vehicles/carrier particles, see FIGS. 79-80. One example is liposomes (shown in FIGS. 79-80) but it can be antibodies-linked-to-any—carrier-with-MetPhyte or MetPhyte PLUS, or/and magnetic particles, or any other type of affinity-based particles for directed delivery of MetPhyte or MetPhyte PLUS;
sub-dermal administration, e.g., via injection of MetPhyte or MetPhyte PLUS (at any frequency of injection, with any suitable device);
sub-dermal administration via implanting of MetPhyte or MetPhyte PLUS in any type of carrier, e.g., PLGA or any other molecule https://en.wikipedia.org/wiki/PLGA, any gel (e.g., but not limited to hydrogel) for slow release of MetPhyte or MetPhyte PLUS (this is especially important for dermal cancer like melanomas, papillomas);
sub-dermal administration via microneedles, e.g., https://www.nature.com/articles/s41551-018-0337-4, where micro-needles may be non-biodegradable or biodegradable, and if biodegradable, they may be completely or partially composed of MetPhyte or MetPhyte PLUS, for slow release of MetPhyte or MetPhyte PLUS into the dermis (this application is suitable for sub-tumor capsule delivery of MetPhyte or MetPhyte PLUS into other type of solid tumors, e.g., brain, but not limited to brain) by applying surgically on a large tumor lump for slow release of MetPhyte or MetPhyte PLUS and inducing tumor cell death. Where the composition of microneedles, if biodegradable, may be partially made from MetPhyte or MetPhyte PLUS or maybe exclusively/preferentially by a PLUS component, while MetPhyte component may be released via these microneedles and coming from a patch itself, or vice versa.
Intra-body administration, for example but not limited to conditions such as gelly-belly, when cancer spreads to internal organs and tissues and the feasible way of treatment is intra-body administration, when a drug is typically embedded into a slowly-dissolving gel, ensheating the internal organs and slowly and consistently delivering the drug to the outside of internal organs and tissues.
via trans-dermal patch
via hypodermic needle
via a topical cream/lotion, e.g., as described here: https://www.sciencedirect.com/science/article/abs/pii/S0141813020353575;
Intra-bone

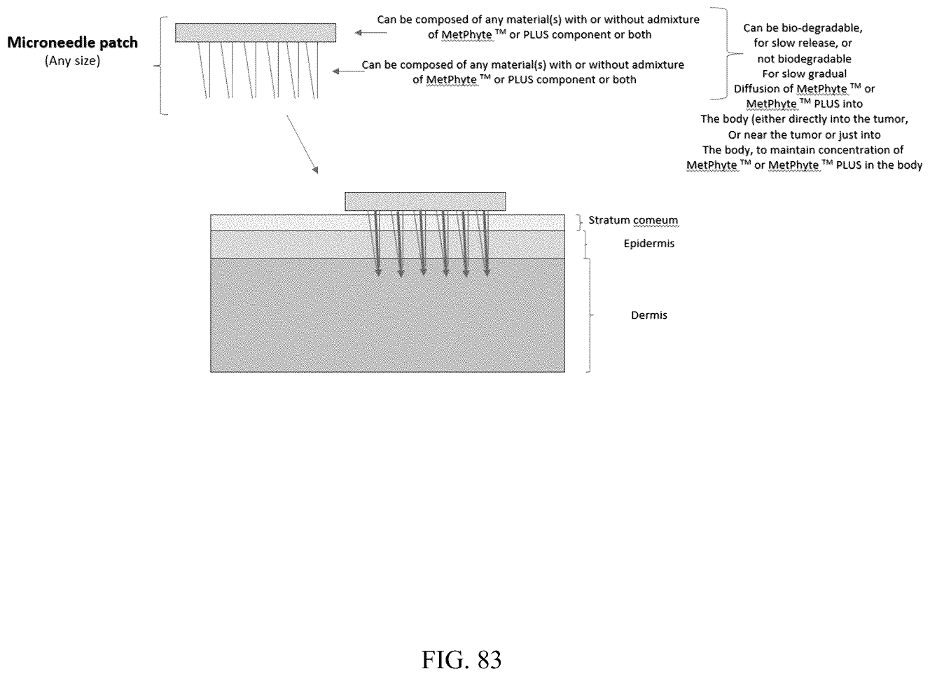

FIG. 83 is a diagram showing one embodiment of application via a microneedle patch.

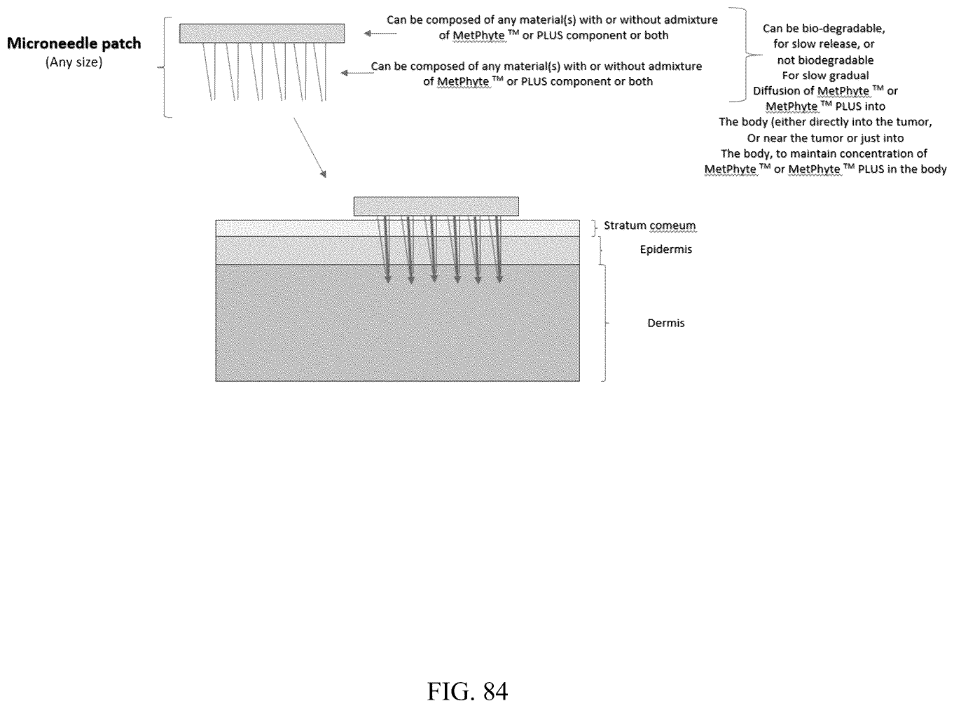

FIG. 84 is a diagram showing one embodiment of application via a microneedle patch.

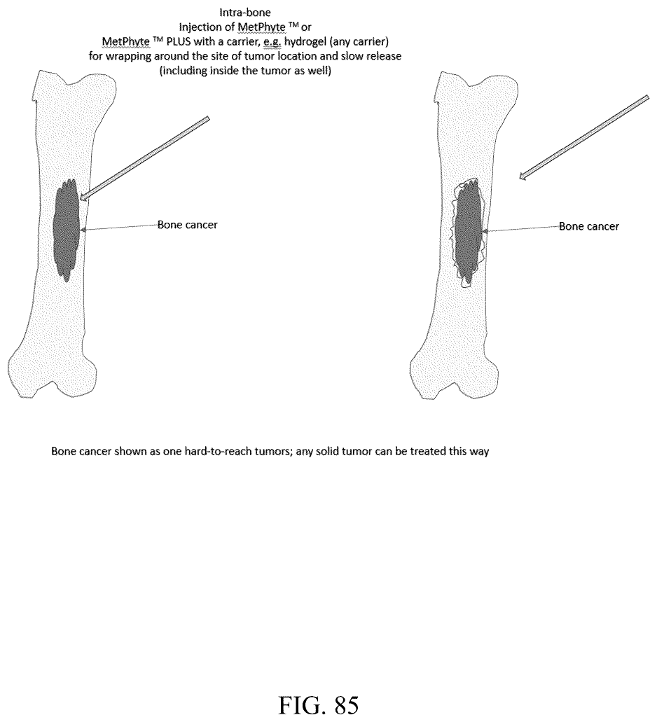

FIG. 85 is a diagram showing one embodiment of application via intra bone injection.

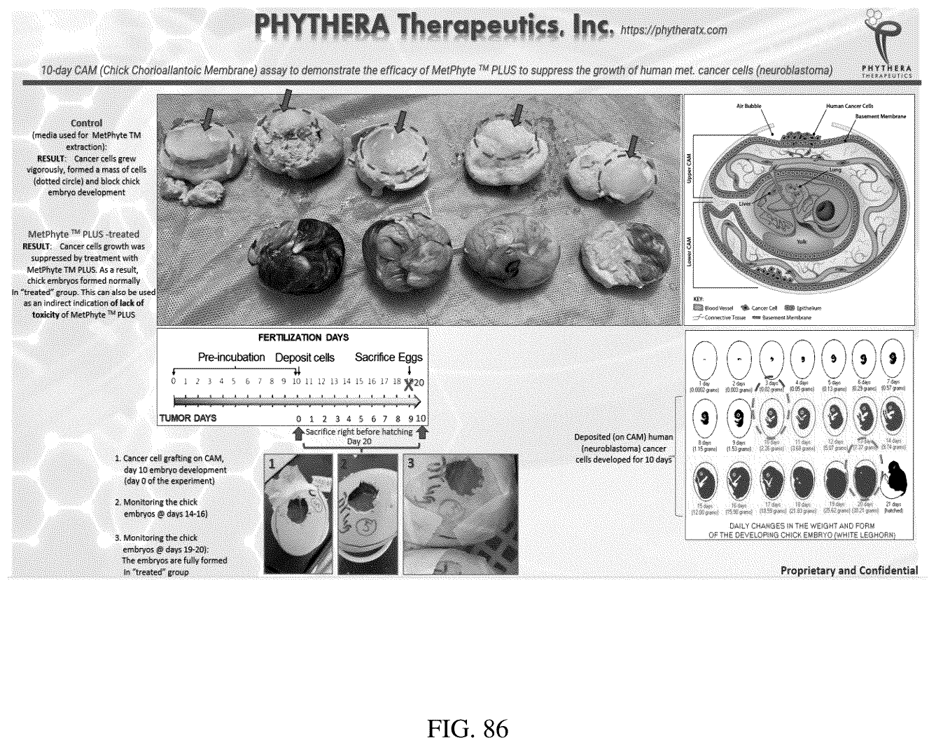

FIG. 86 is a diagram showing one embodiment of application of MetPhyte Plus on chicken embryos.

Data on in ovo model (CAM model, chick chorioallantoic membrane assay model), where we injected tumor cells into the egg and showed that MetPhyte PLUS can efficiently kill human tumor cells-is shown in FIG. 86.

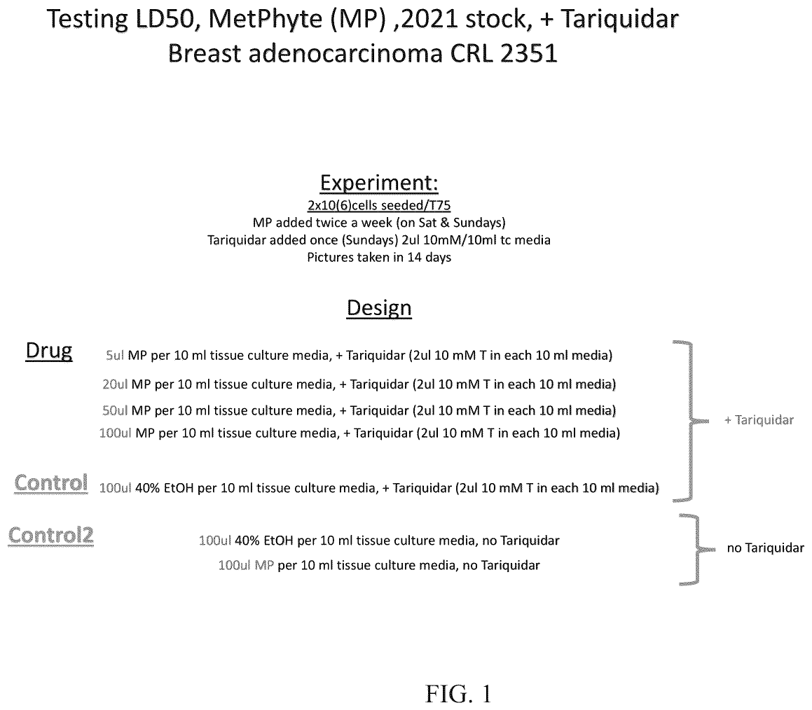

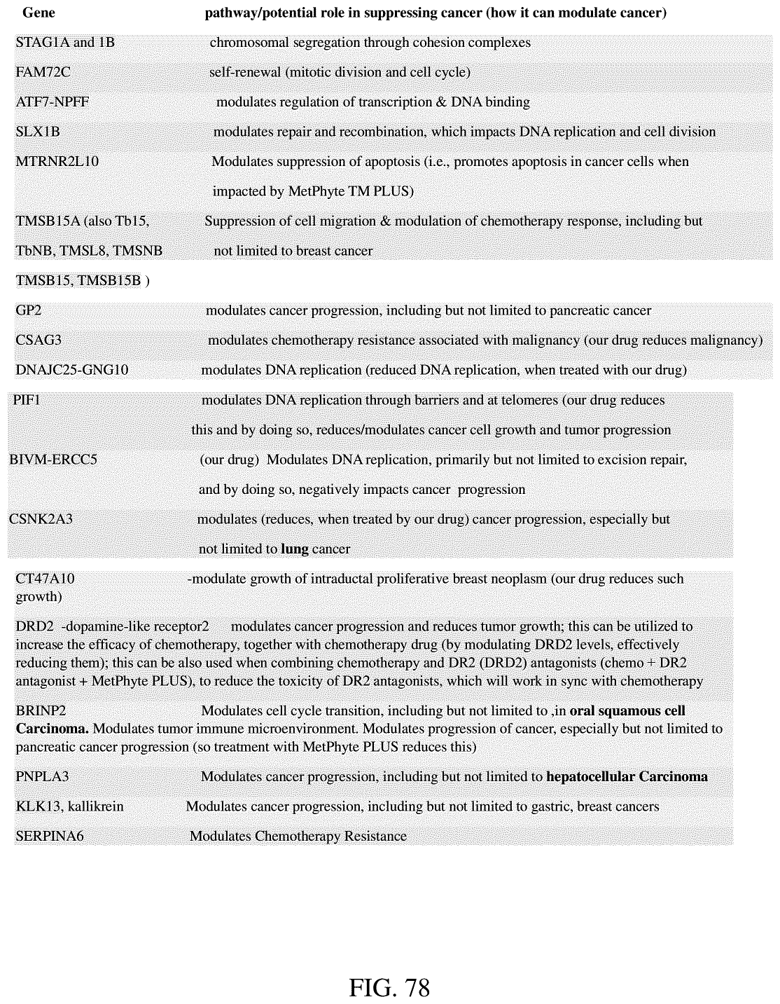

FIGS. 1-78 provide additional information, including cell models, efficacy, and study results.

FIG. 1 shows an outline for an experiment for testing one embodiment of MetPhyte and Tariquidar.

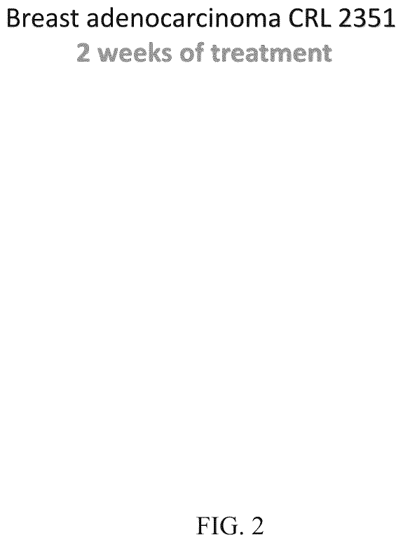

FIG. 2 shows a condition for an experiment for testing one embodiment of MetPhyte and Tariquidar.

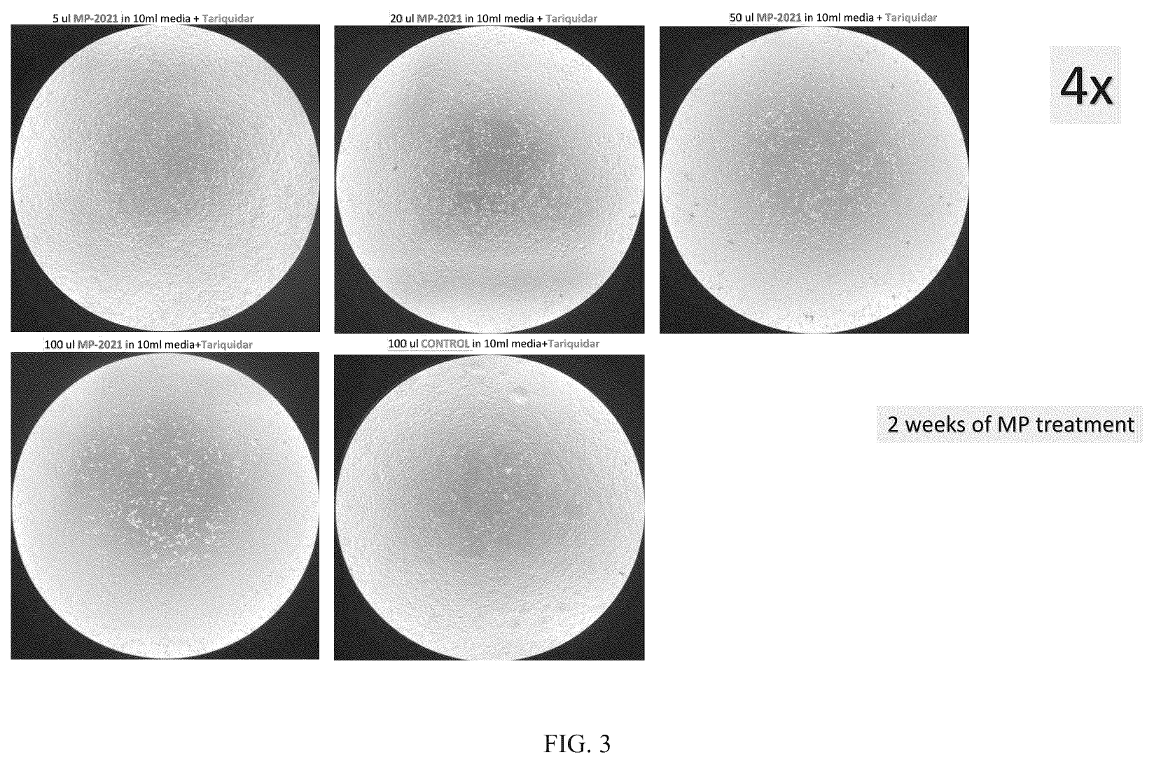

FIG. 3 shows the progress of one experiment for testing one embodiment of MetPhyte and Tariquidar after 2 weeks.

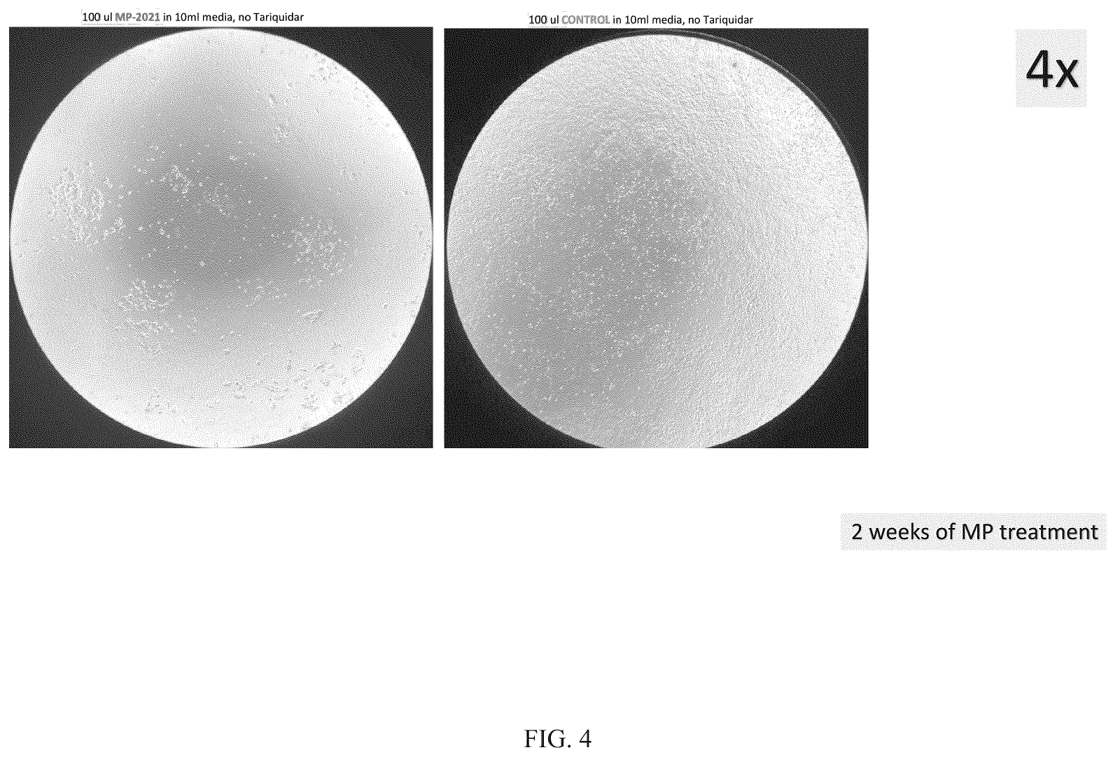

FIG. 4 shows the progress of one experiment for testing one embodiment of MetPhyte and Tariquidar after 2 weeks.

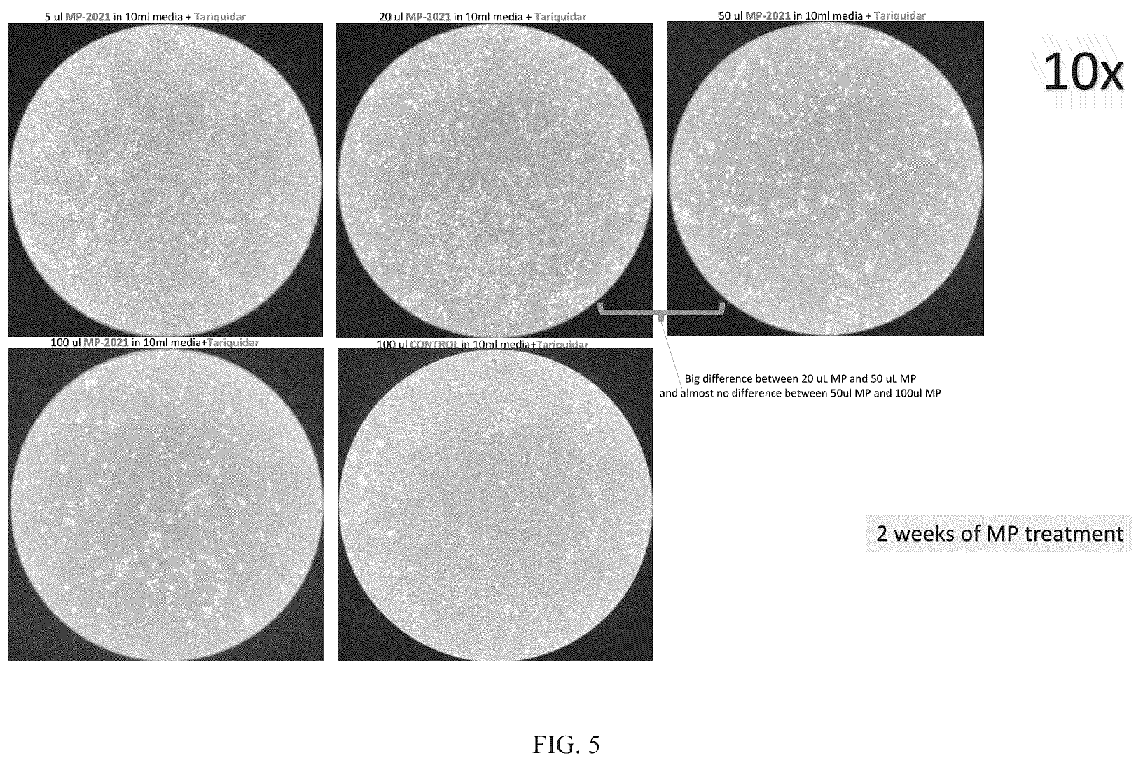

FIG. 5 shows the progress of one experiment for testing one embodiment of MetPhyte and Tariquidar after 2 weeks.

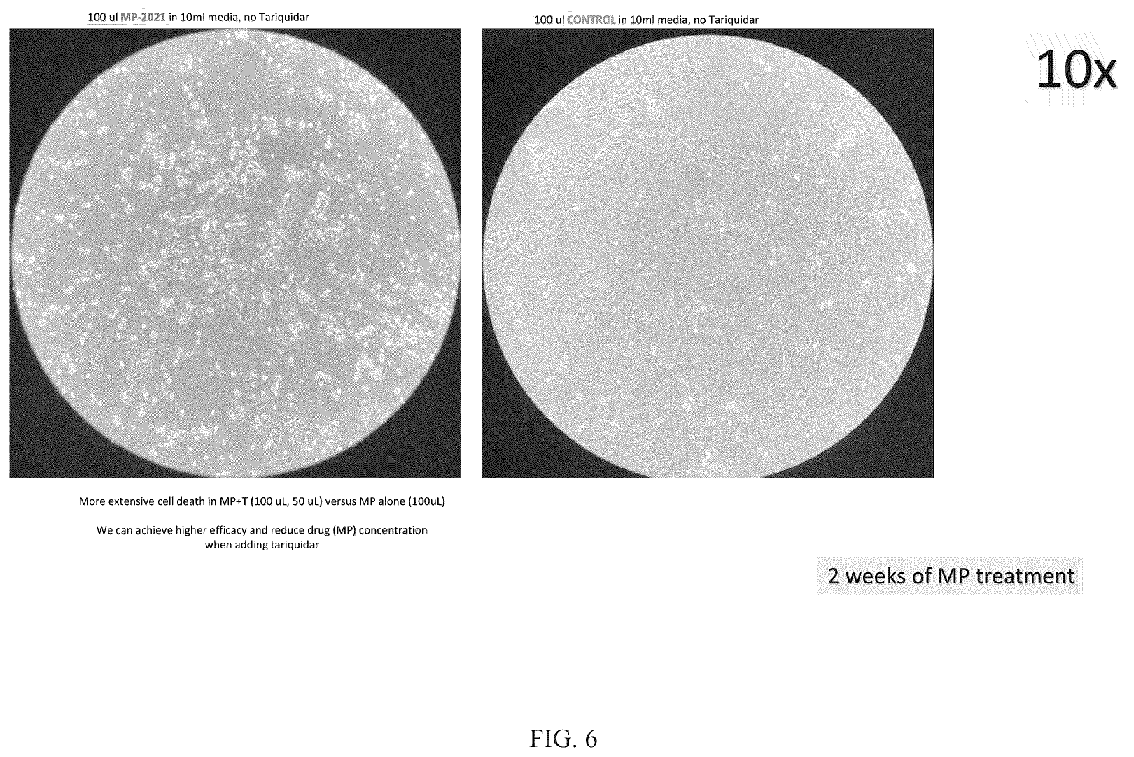

FIG. 6 shows the progress of one experiment for testing one embodiment of MetPhyte and Tariquidar after 2 weeks.

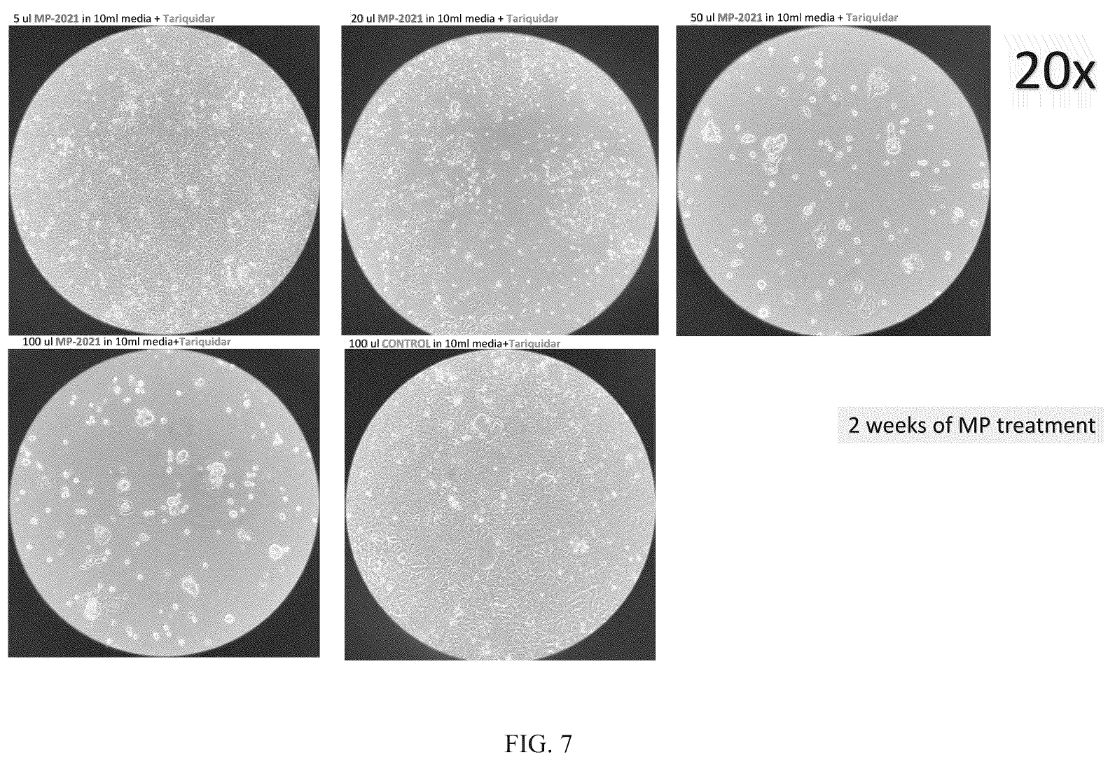

FIG. 7 shows the progress of one experiment for testing one embodiment of MetPhyte and Tariquidar after 2 weeks.

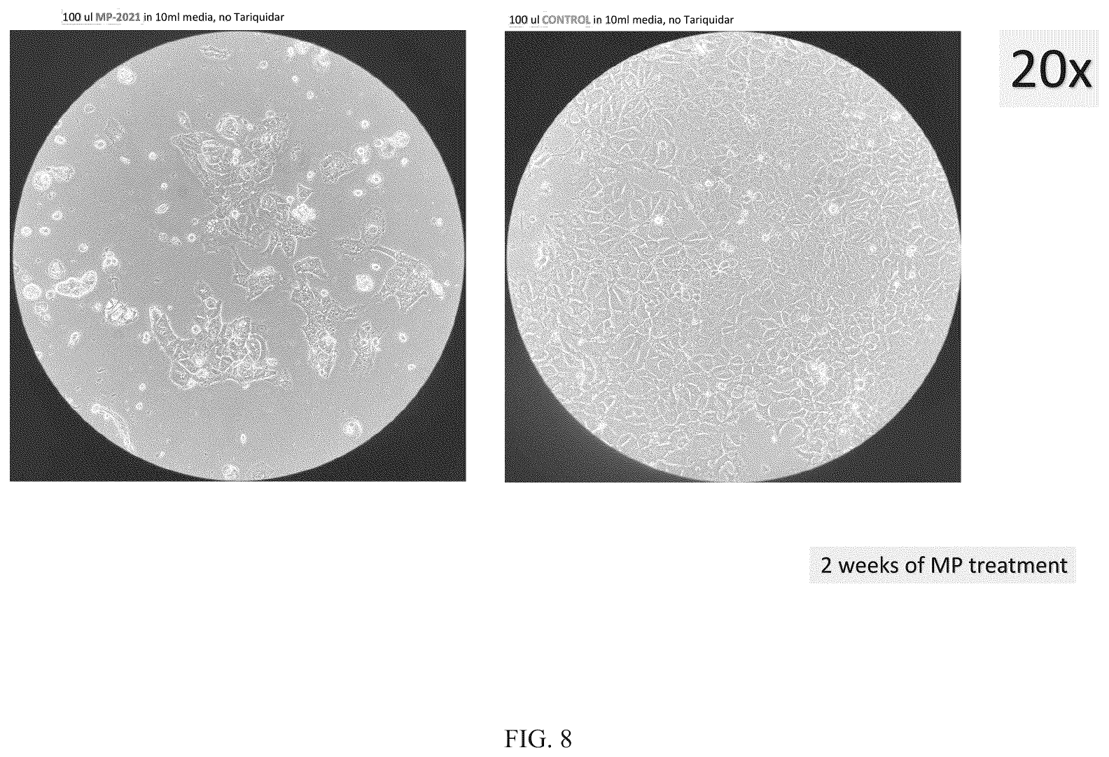

FIG. 8 shows the progress of one experiment for testing one embodiment of MetPhyte and Tariquidar after 2 weeks.

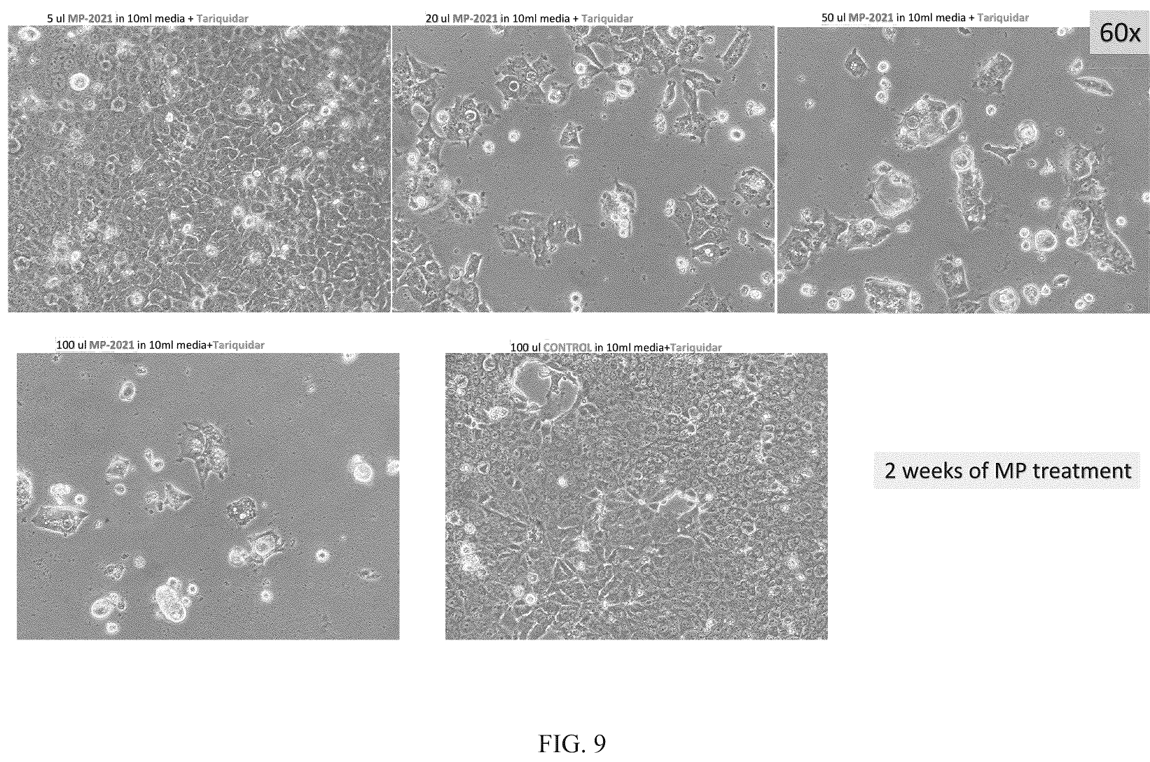

FIG. 9 shows the progress of one experiment for testing one embodiment of MetPhyte and Tariquidar after 2 weeks.

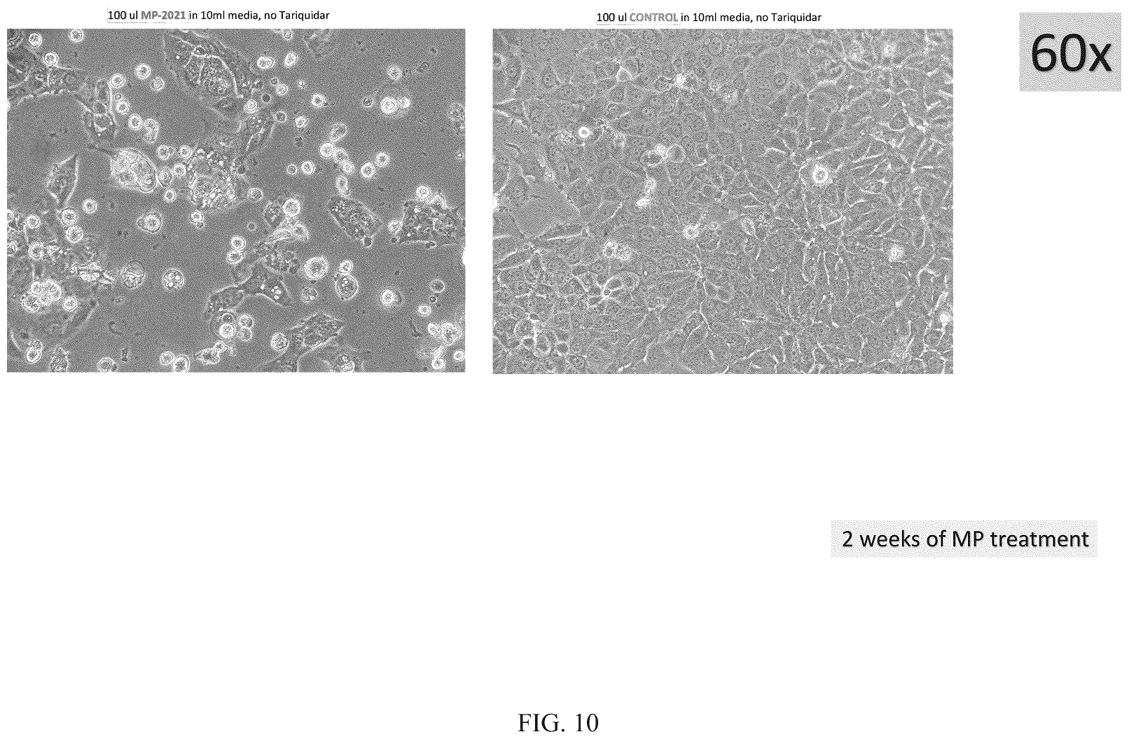

FIG. 10 shows the progress of one experiment for testing one embodiment of MetPhyte and Tariquidar after 2 weeks.

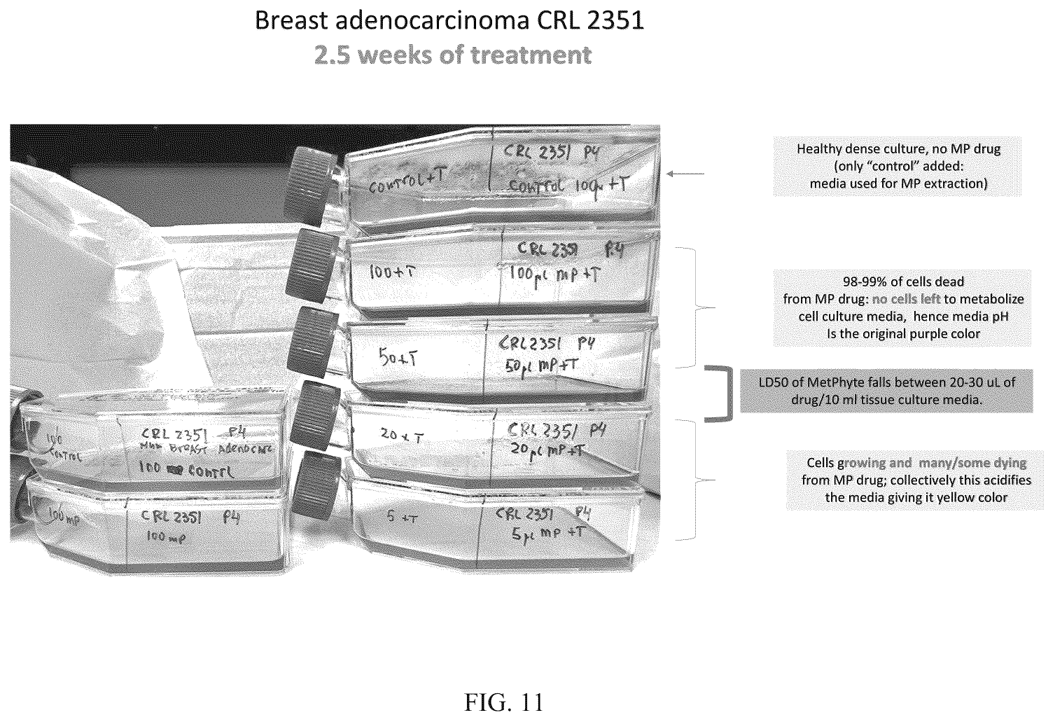

FIG. 11 shows the progress of one experiment for testing one embodiment of MetPhyte and Tariquidar after 2.5 weeks.

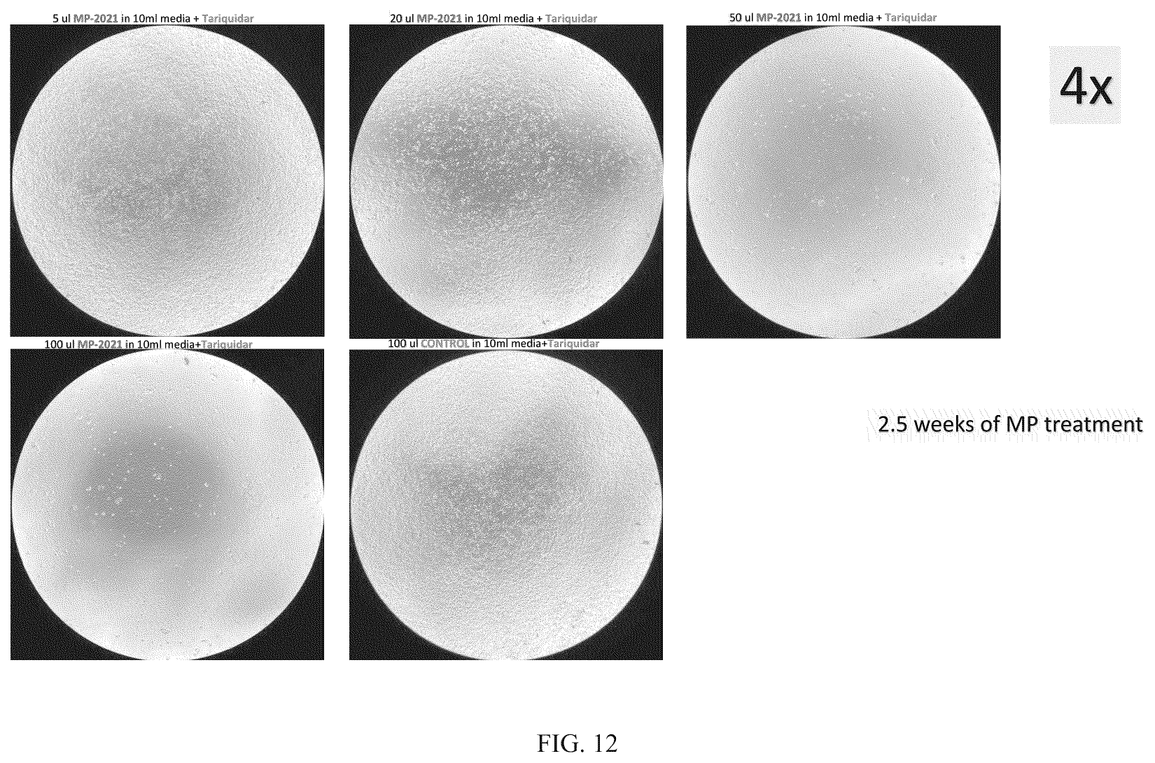

FIG. 12 shows the progress of one experiment for testing one embodiment of MetPhyte and Tariquidar after 2.5 weeks.

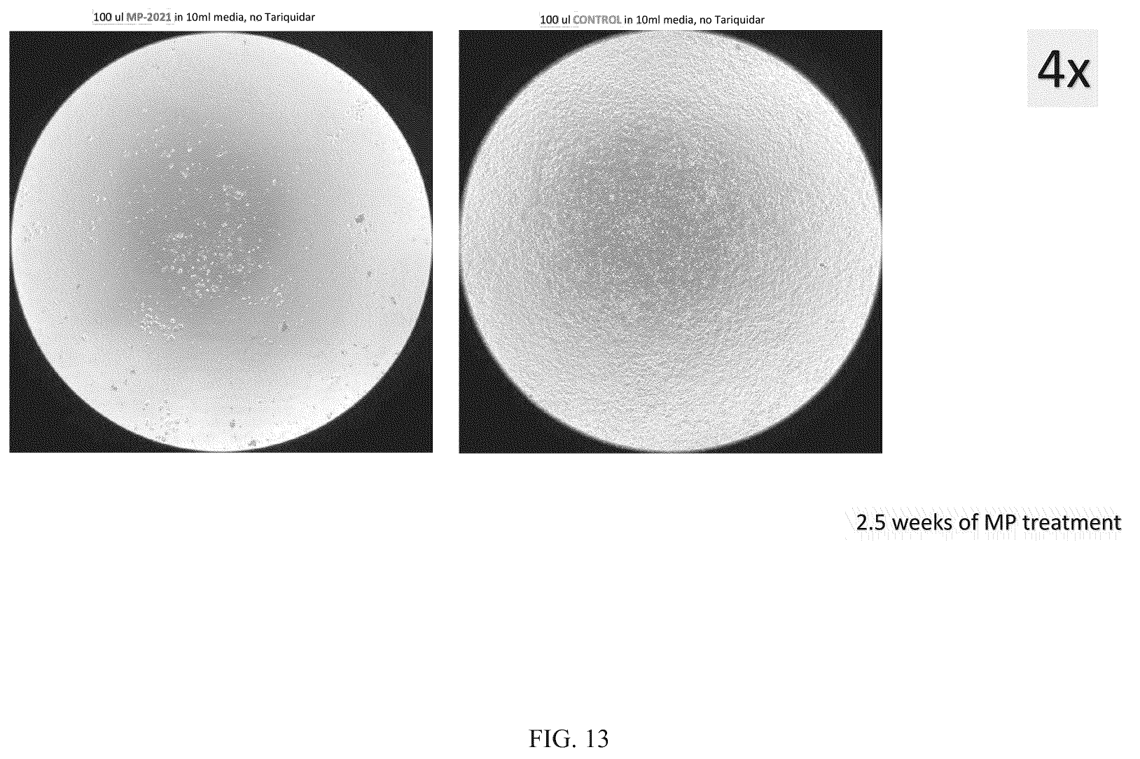

FIG. 13 shows the progress of one experiment for testing one embodiment of MetPhyte and Tariquidar after 2.5 weeks.

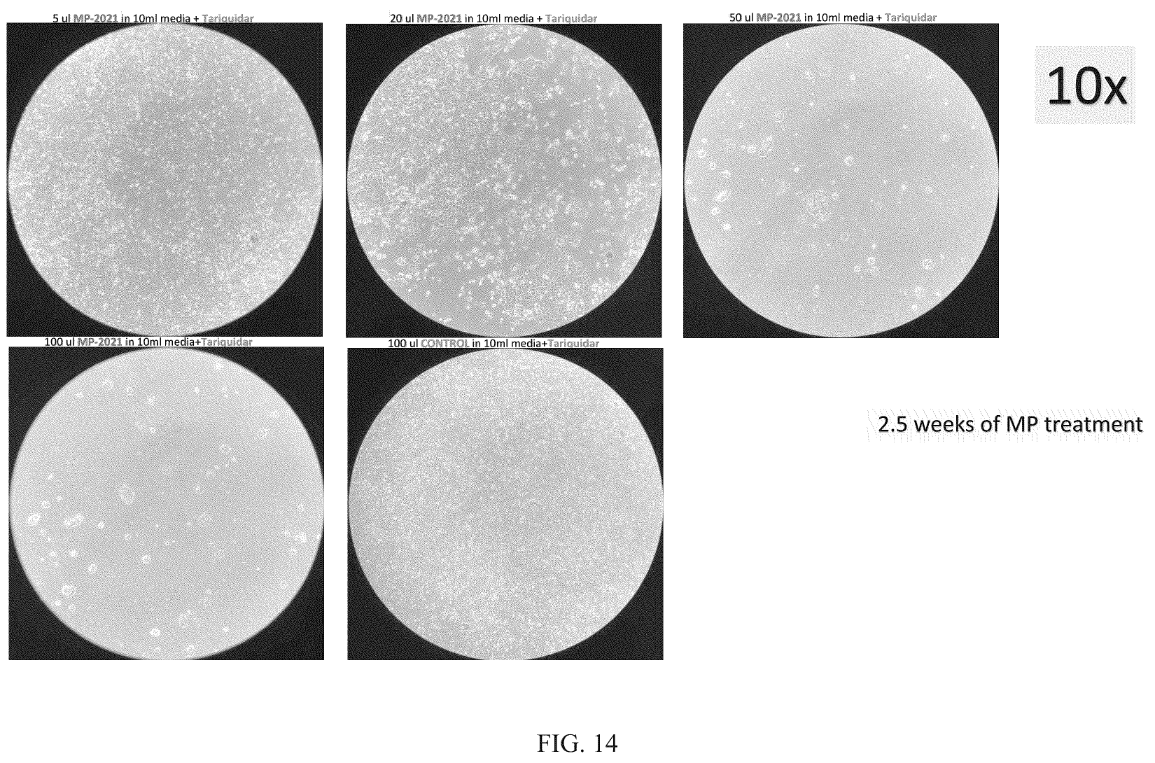

FIG. 14 shows the progress of one experiment for testing one embodiment of MetPhyte and Tariquidar after 2.5 weeks.

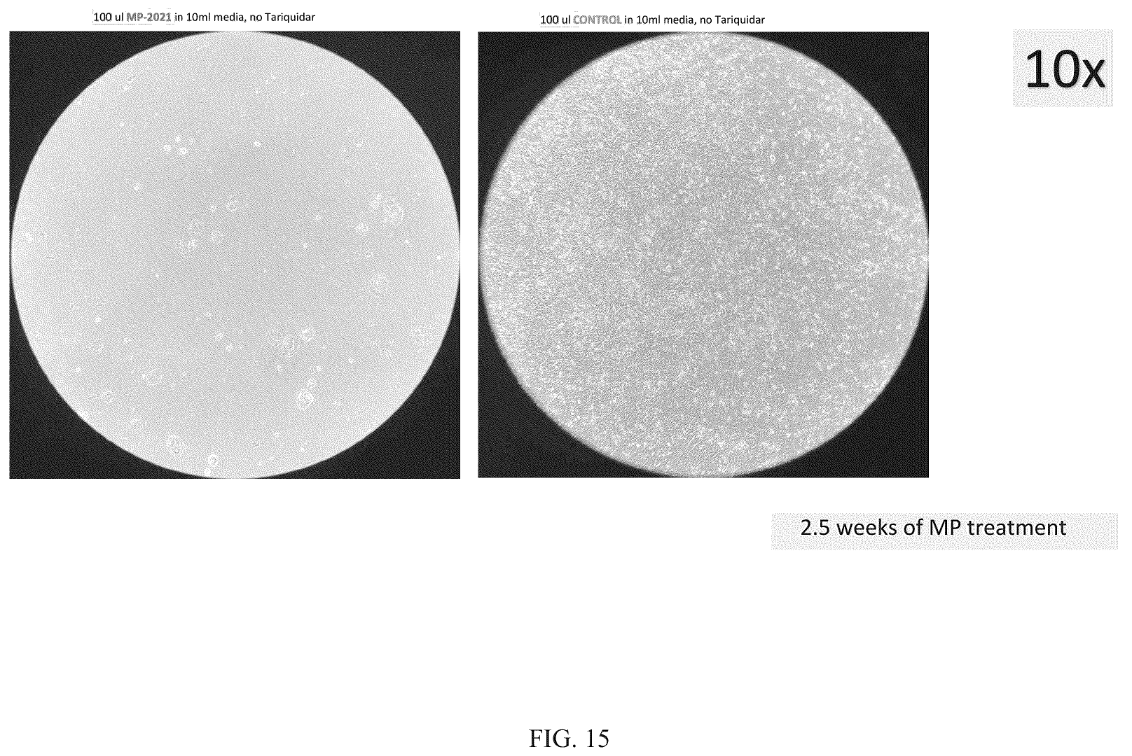

FIG. 15 shows the progress of one experiment for testing one embodiment of MetPhyte and Tariquidar after 2.5 weeks.

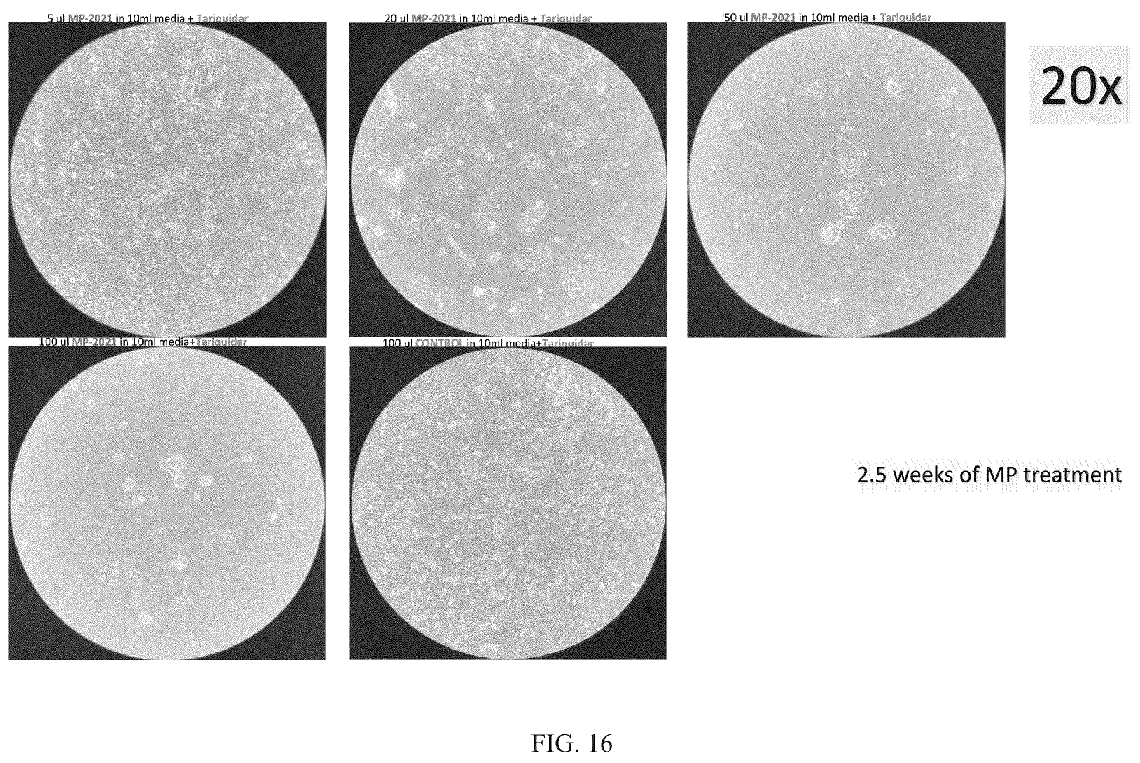

FIG. 16 shows the progress of one experiment for testing one embodiment of MetPhyte and Tariquidar after 2.5 weeks.

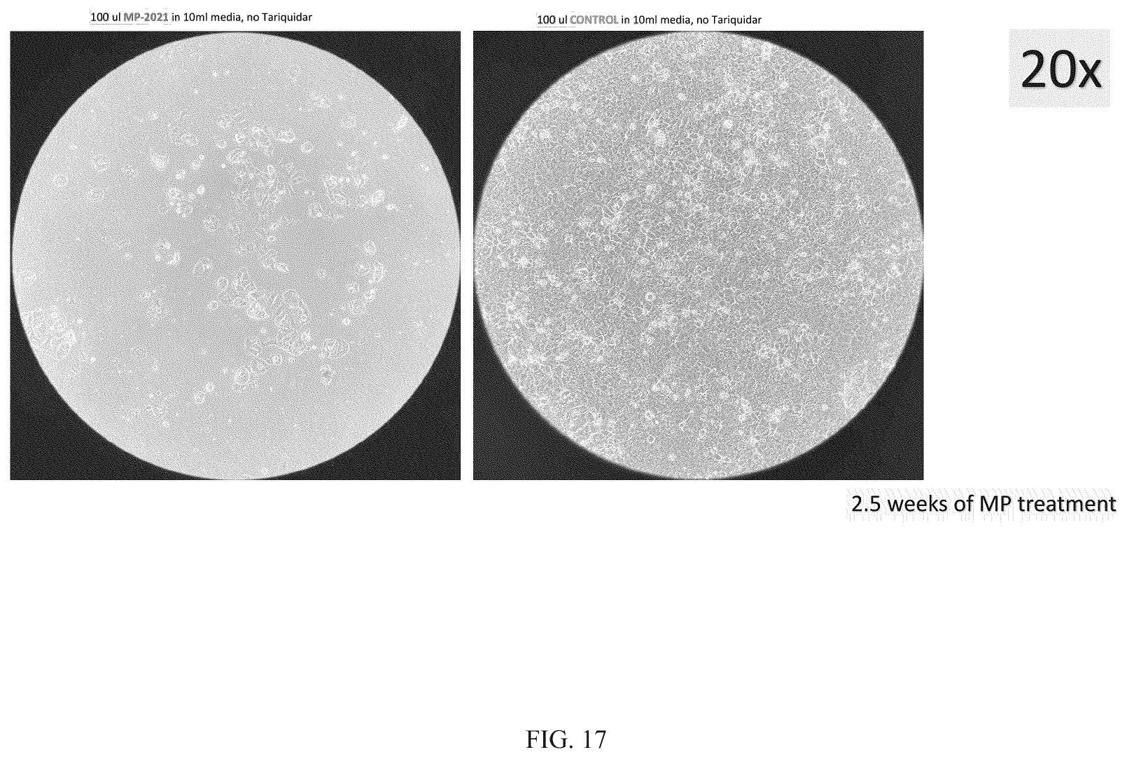

FIG. 17 shows the progress of one experiment for testing one embodiment of MetPhyte and Tariquidar after 2.5 weeks.

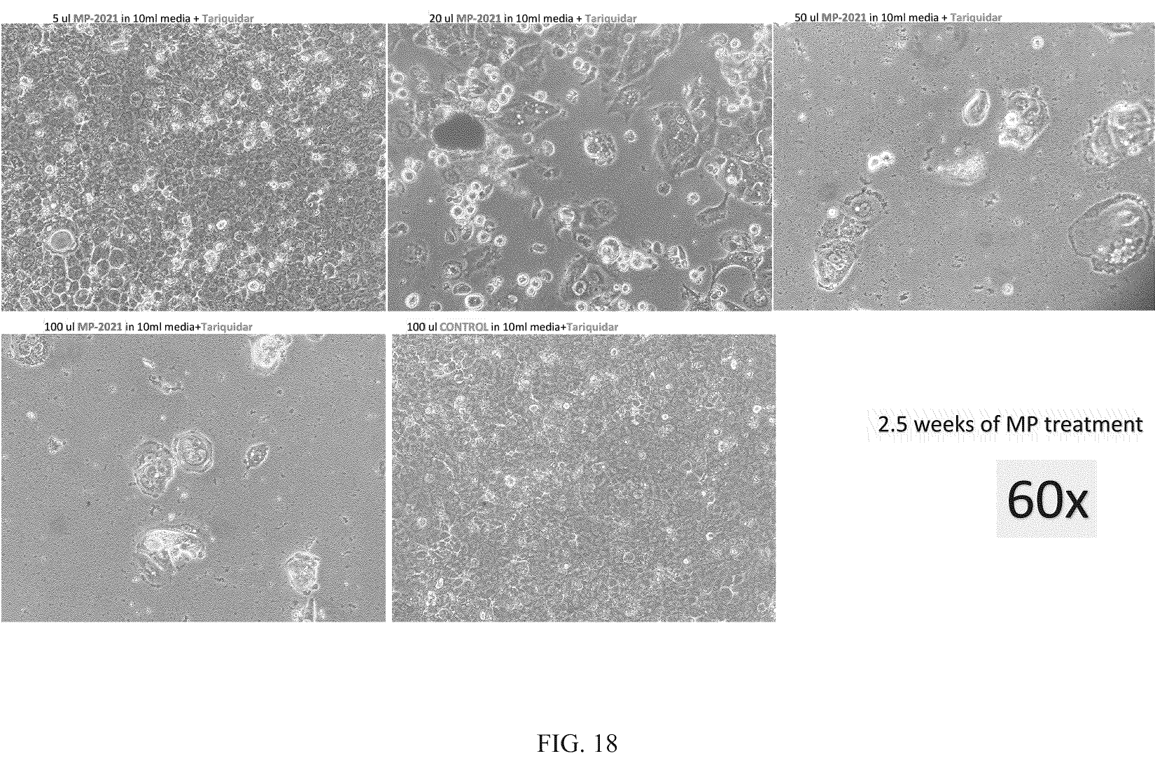

FIG. 18 shows the progress of one experiment for testing one embodiment of MetPhyte and Tariquidar after 2.5 weeks.

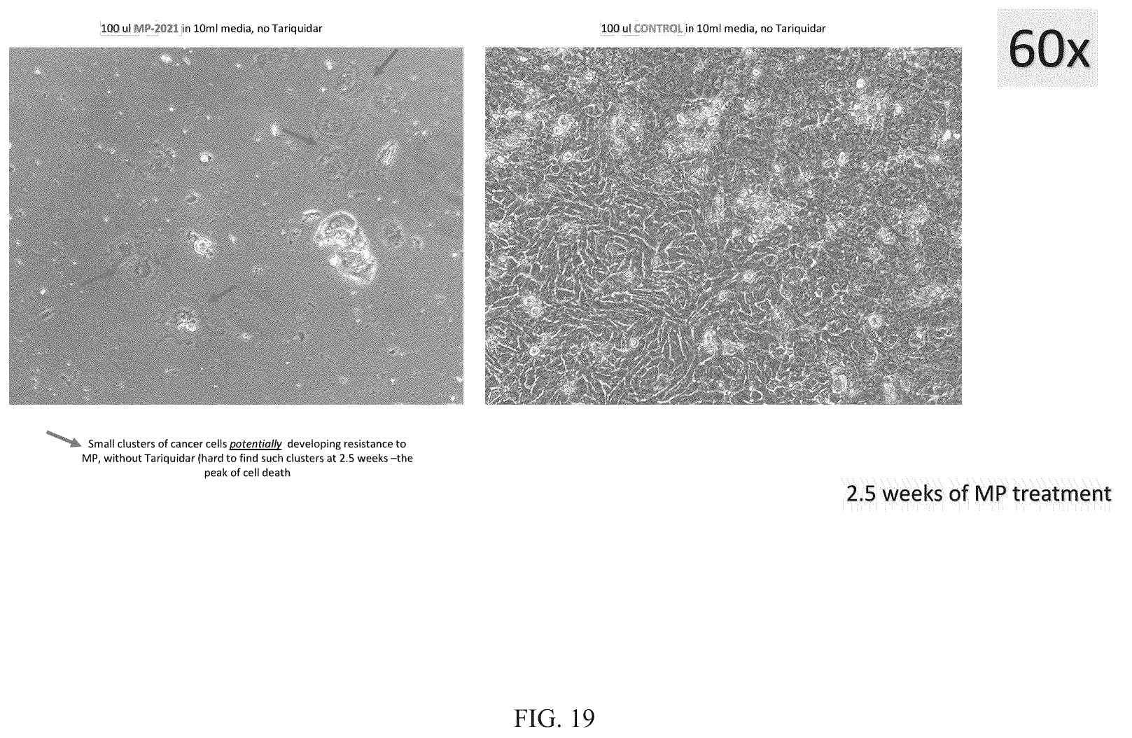

FIG. 19 shows the progress of one experiment for testing one embodiment of MetPhyte and Tariquidar after 2.5 weeks.

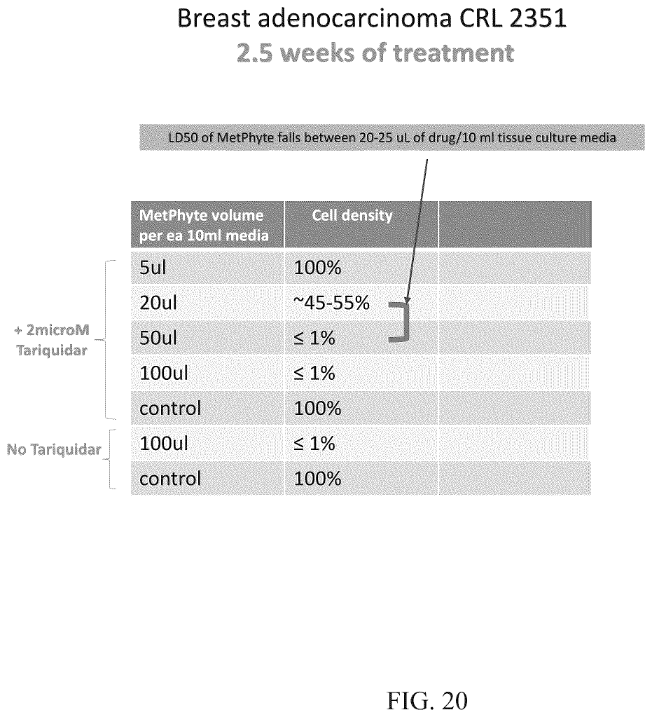

FIG. 20 shows the progress of one experiment for testing one embodiment of MetPhyte and Tariquidar after 2.5 weeks.

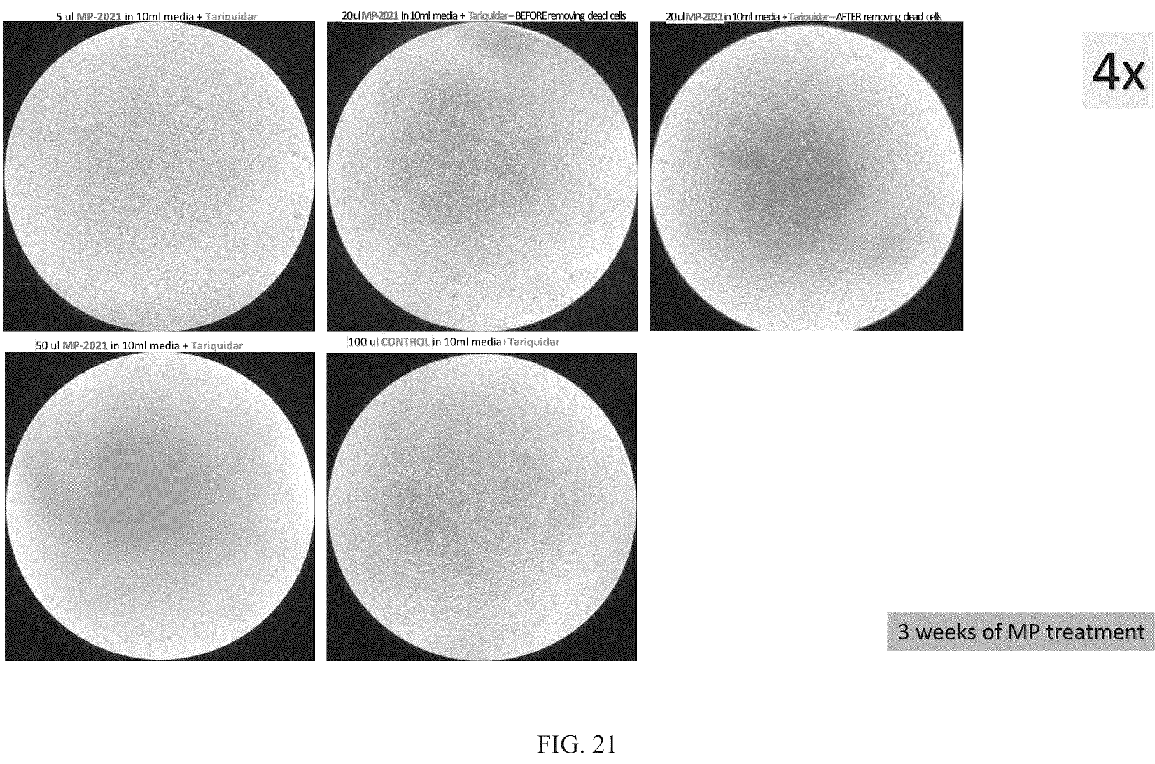

FIG. 21 shows the progress of one experiment for testing one embodiment of MetPhyte and Tariquidar after 3 weeks.

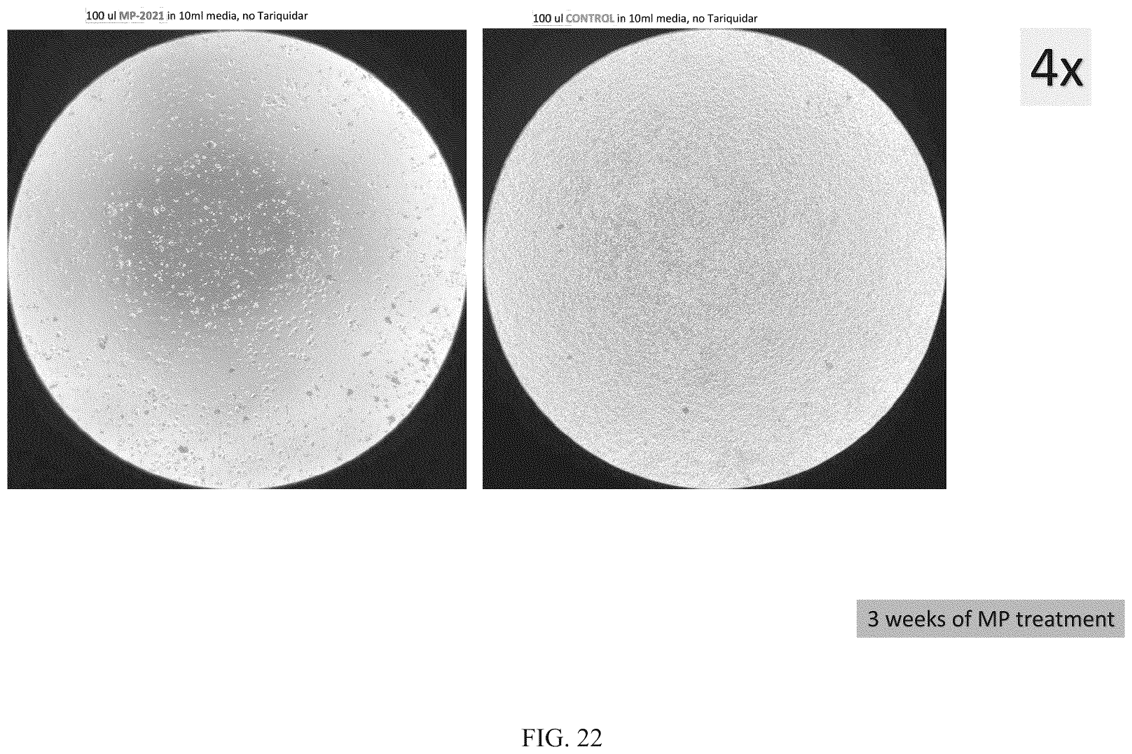

FIG. 22 shows the progress of one experiment for testing one embodiment of MetPhyte and Tariquidar after 3 weeks.

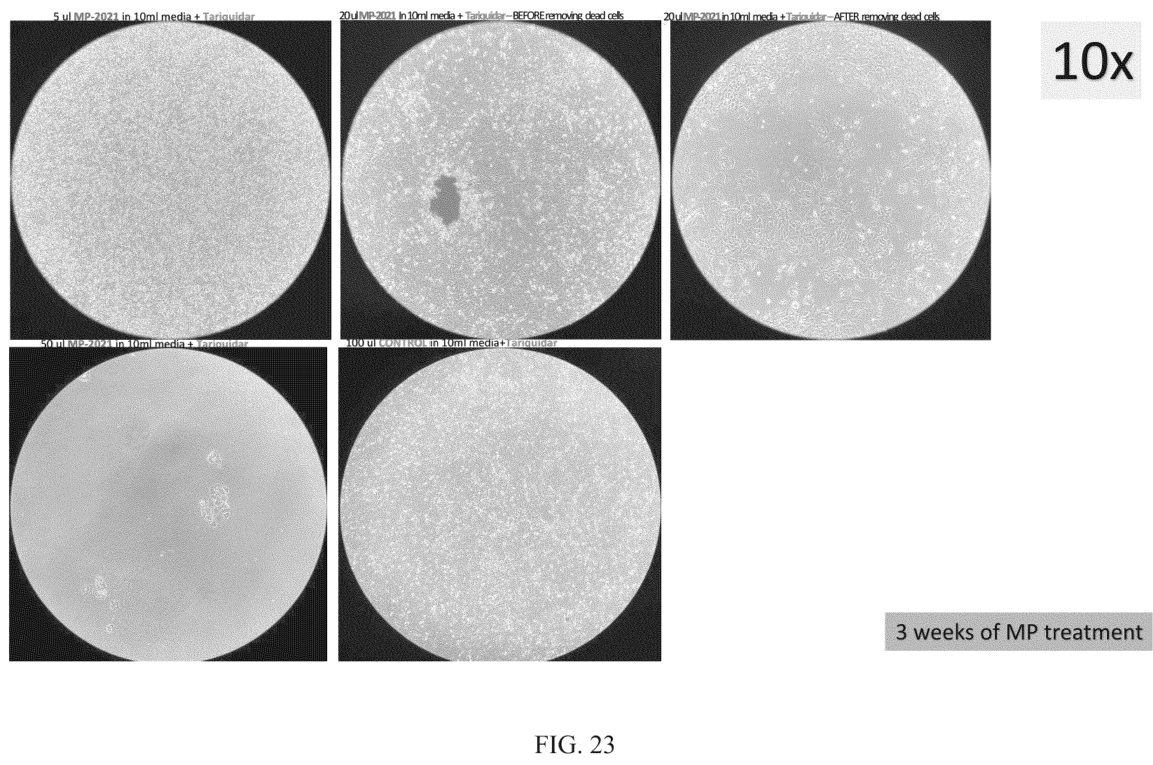

FIG. 23 shows the progress of one experiment for testing one embodiment of MetPhyte and Tariquidar after 3 weeks.

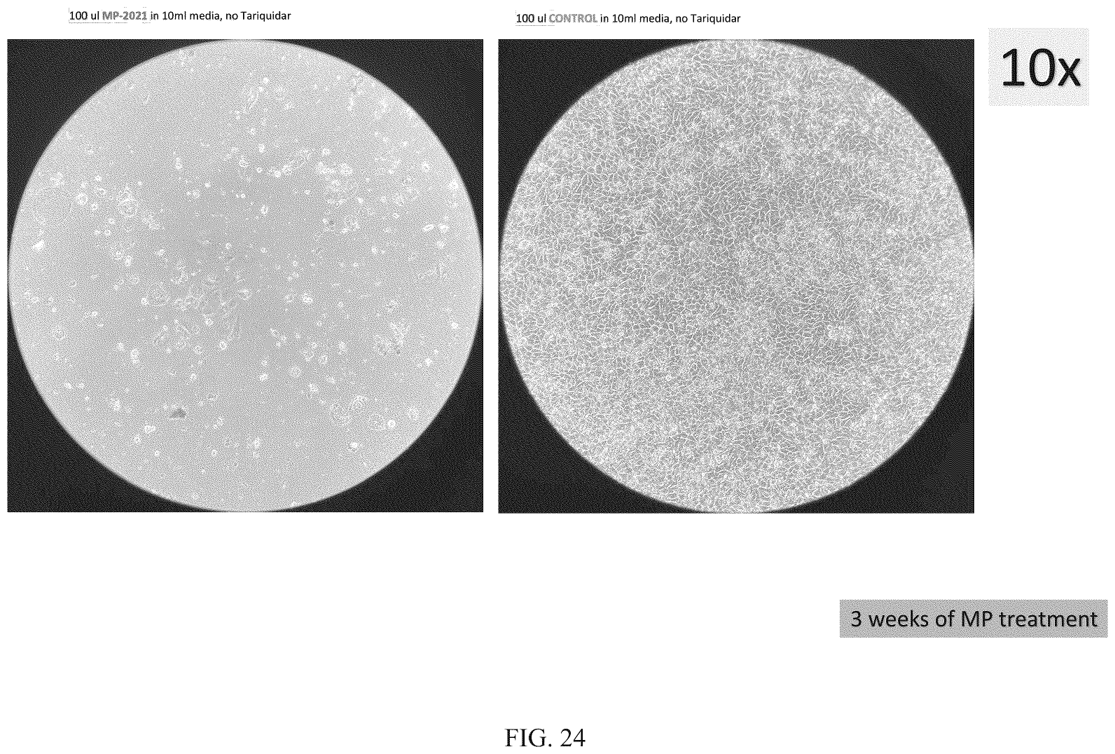

FIG. 24 shows the progress of one experiment for testing one embodiment of MetPhyte and Tariquidar after 3 weeks.

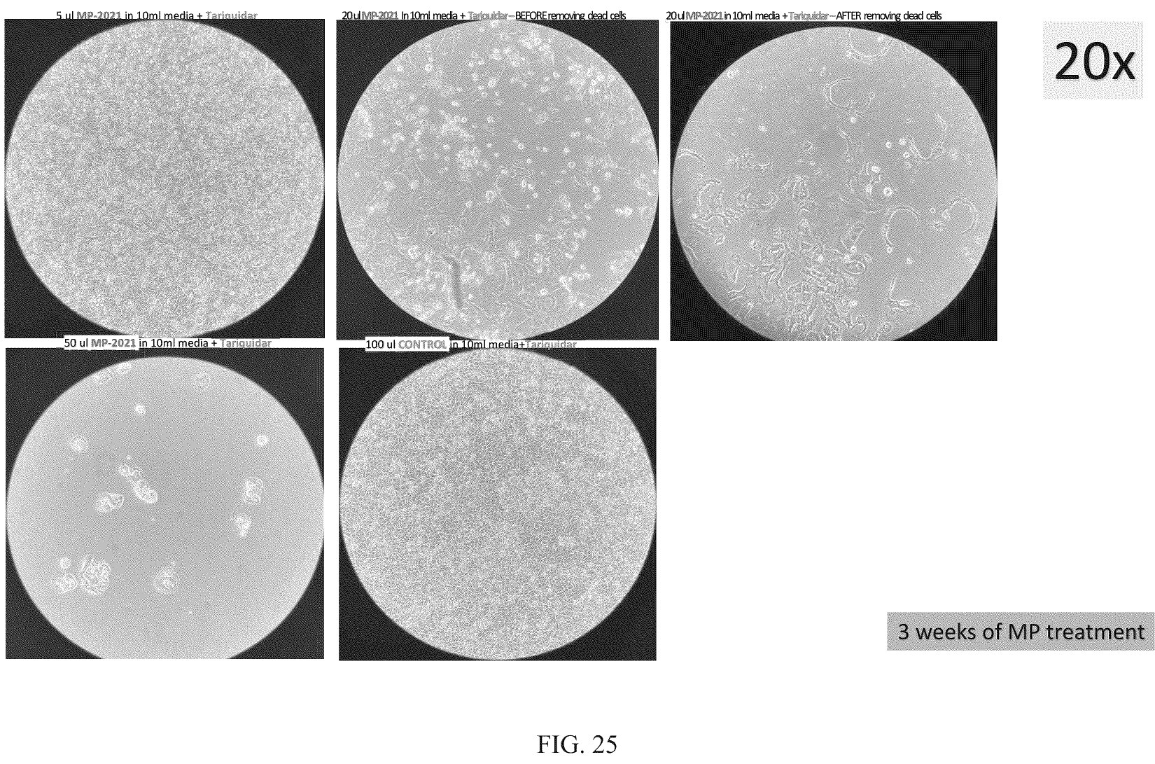

FIG. 25 shows the progress of one experiment for testing one embodiment of MetPhyte and Tariquidar after 3 weeks.

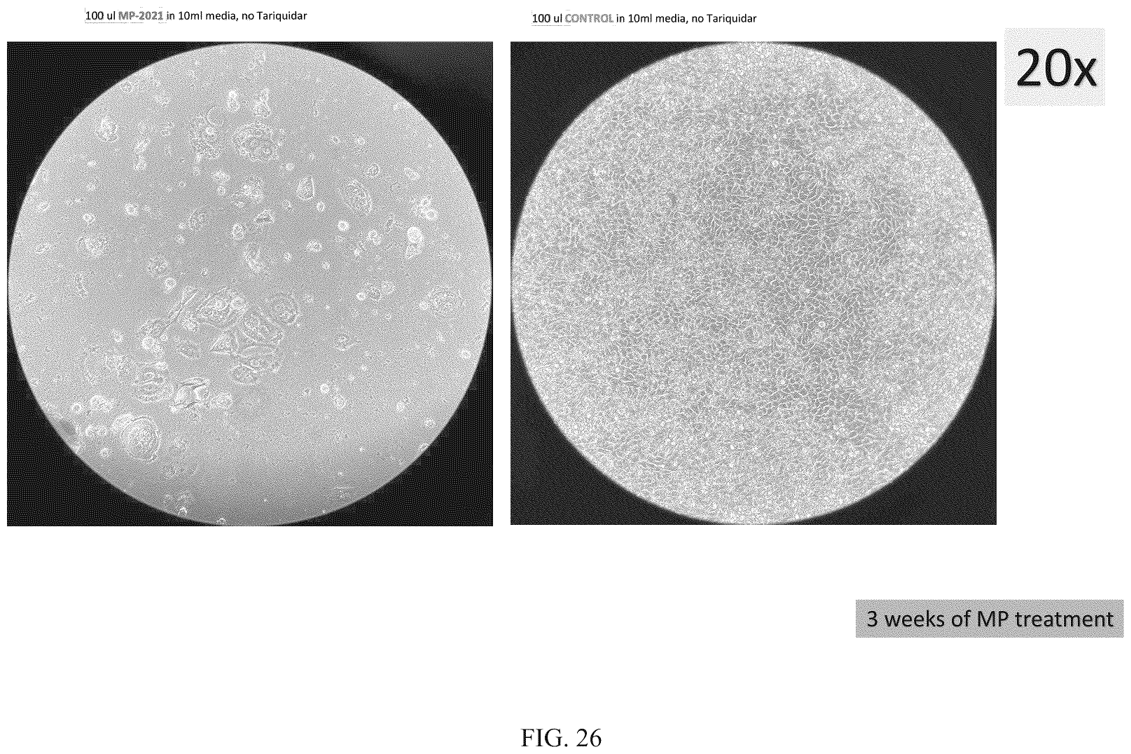

FIG. 26 shows the progress of one experiment for testing one embodiment of MetPhyte and Tariquidar after 3 weeks.

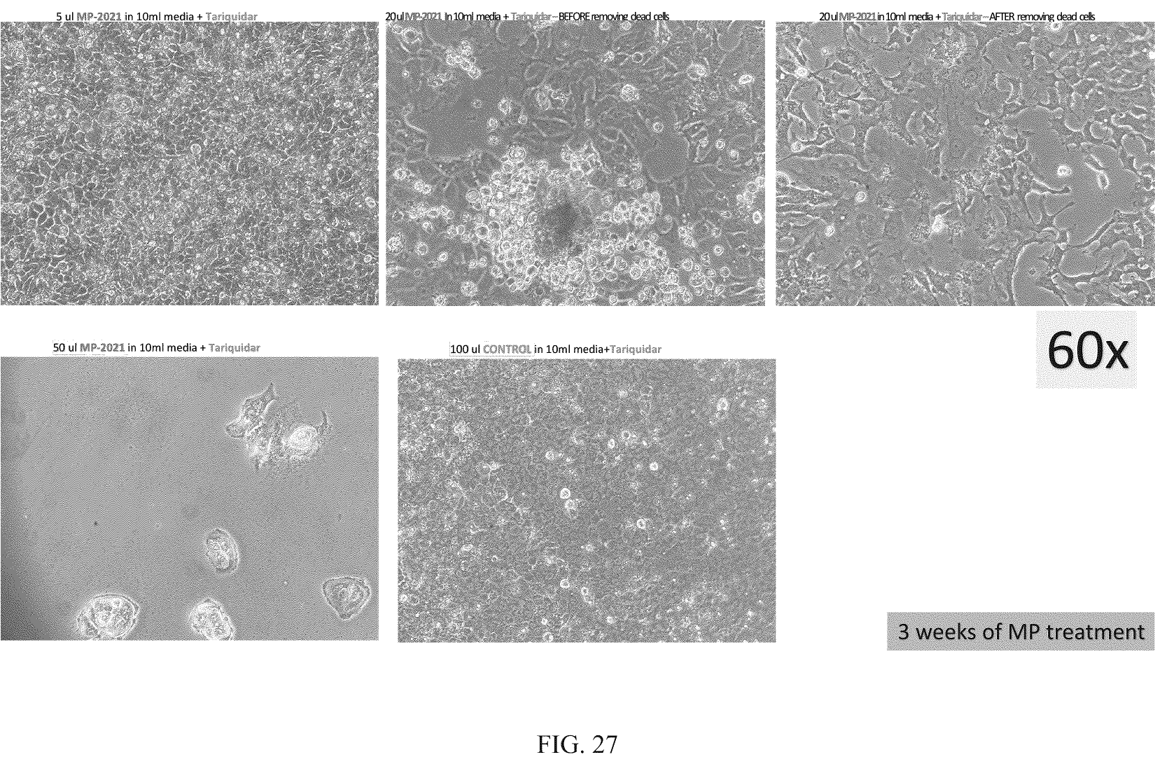

FIG. 27 shows the progress of one experiment for testing one embodiment of MetPhyte and Tariquidar after 3 weeks.

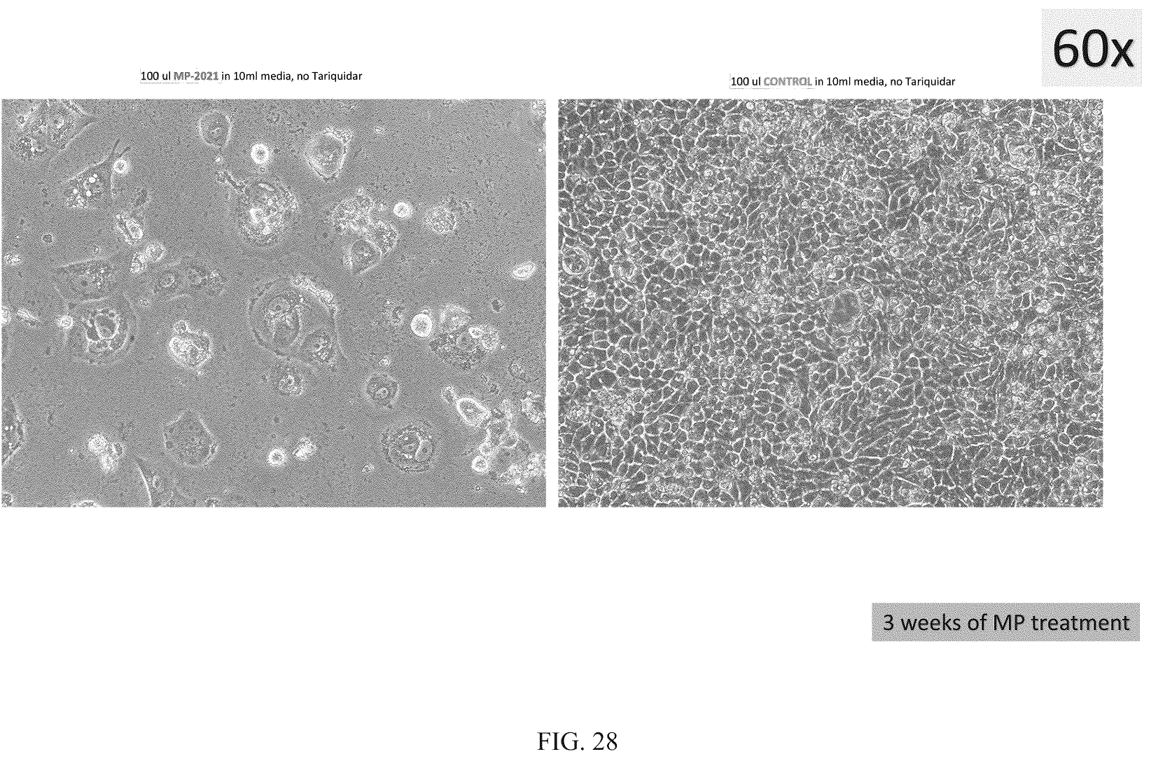

FIG. 28 shows the progress of one experiment for testing one embodiment of MetPhyte and Tariquidar after 3 weeks.

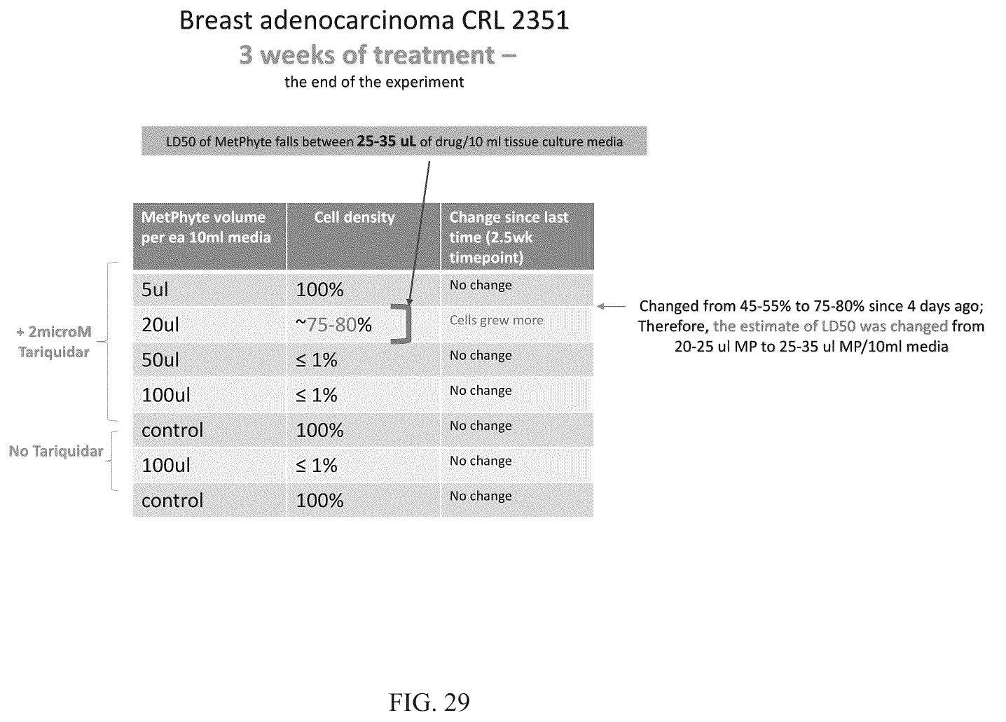

FIG. 29 shows the progress of one experiment for testing one embodiment of MetPhyte and Tariquidar after 3 weeks.

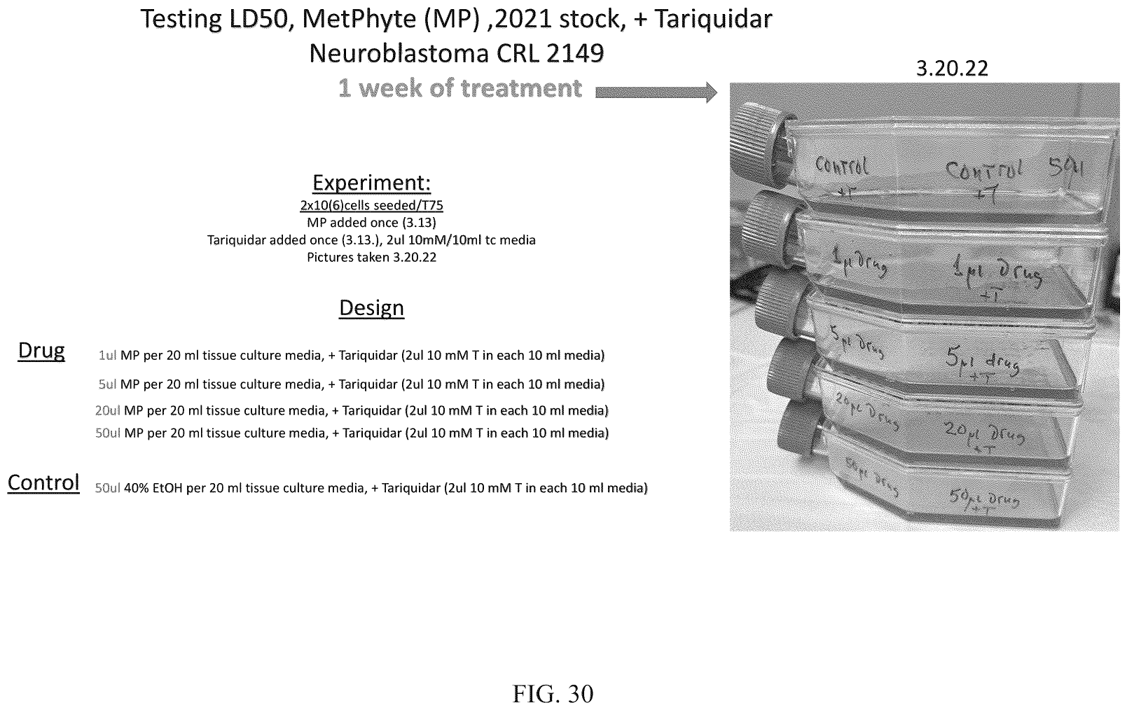

FIG. 30 shows an outline for an experiment for testing one embodiment of MetPhyte and Tariquidar.

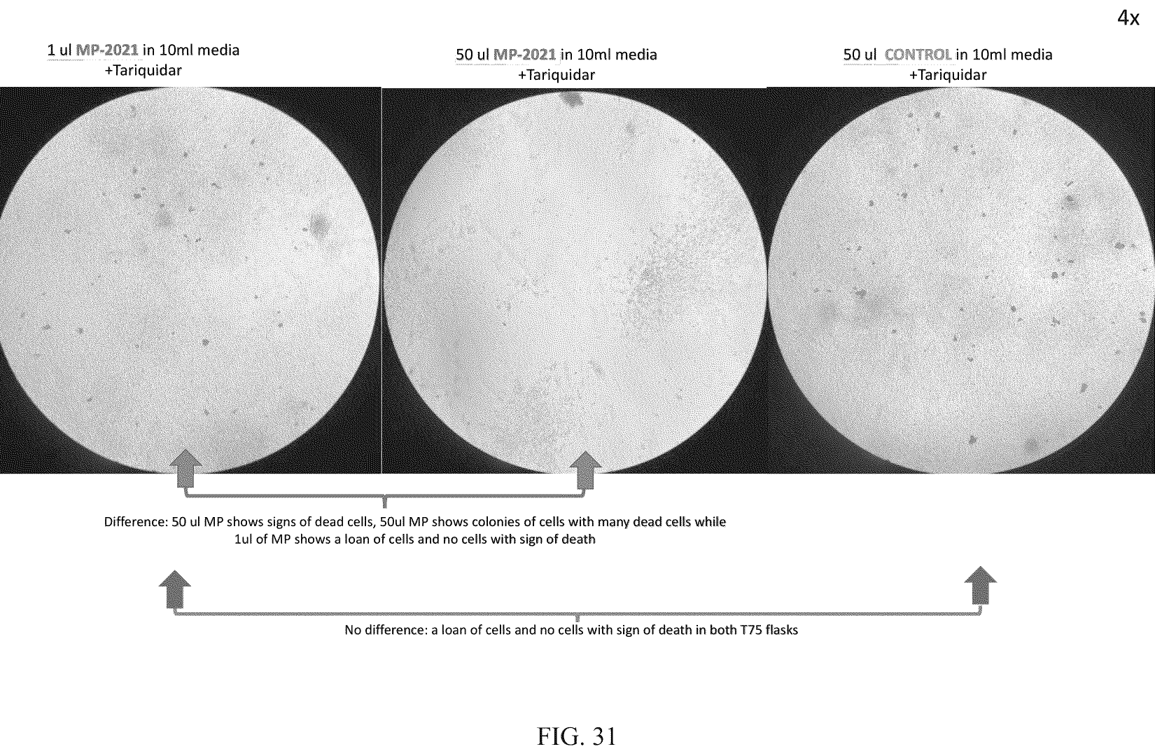

FIG. 31 shows the progress of one experiment for testing one embodiment of MetPhyte and Tariquidar with different amounts of MetPhyte.

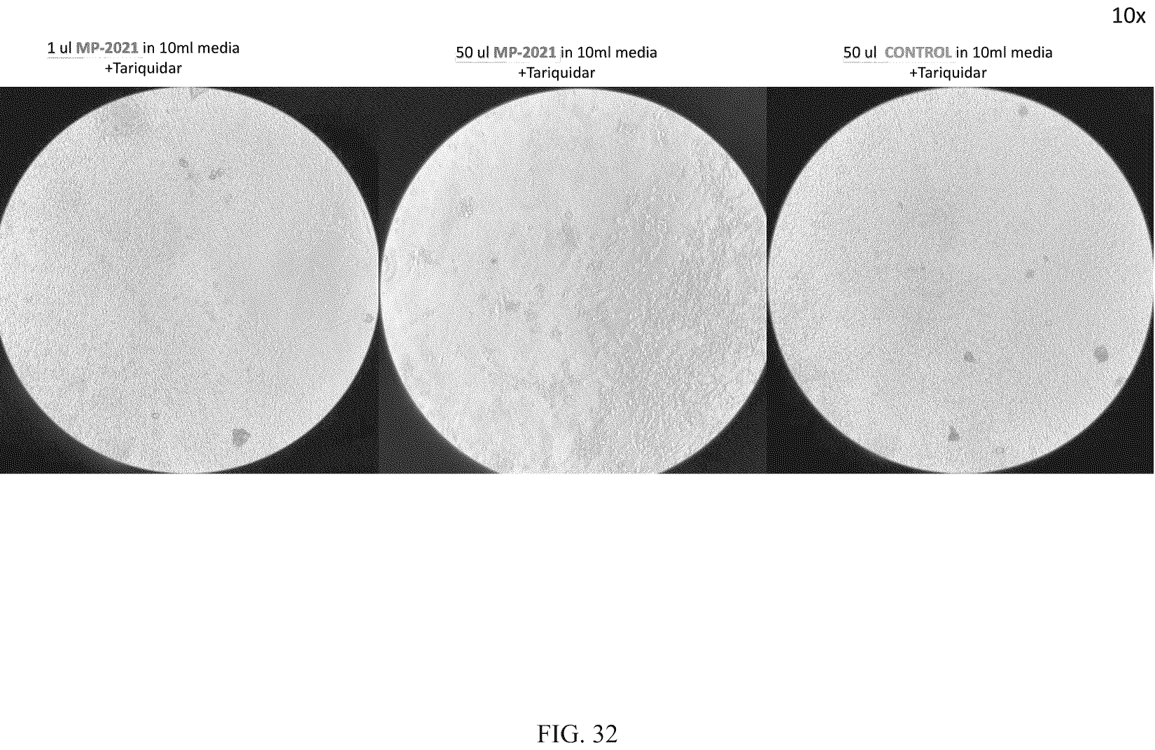

FIG. 32 shows the progress of one experiment for testing one embodiment of MetPhyte and Tariquidar with different amounts of MetPhyte.

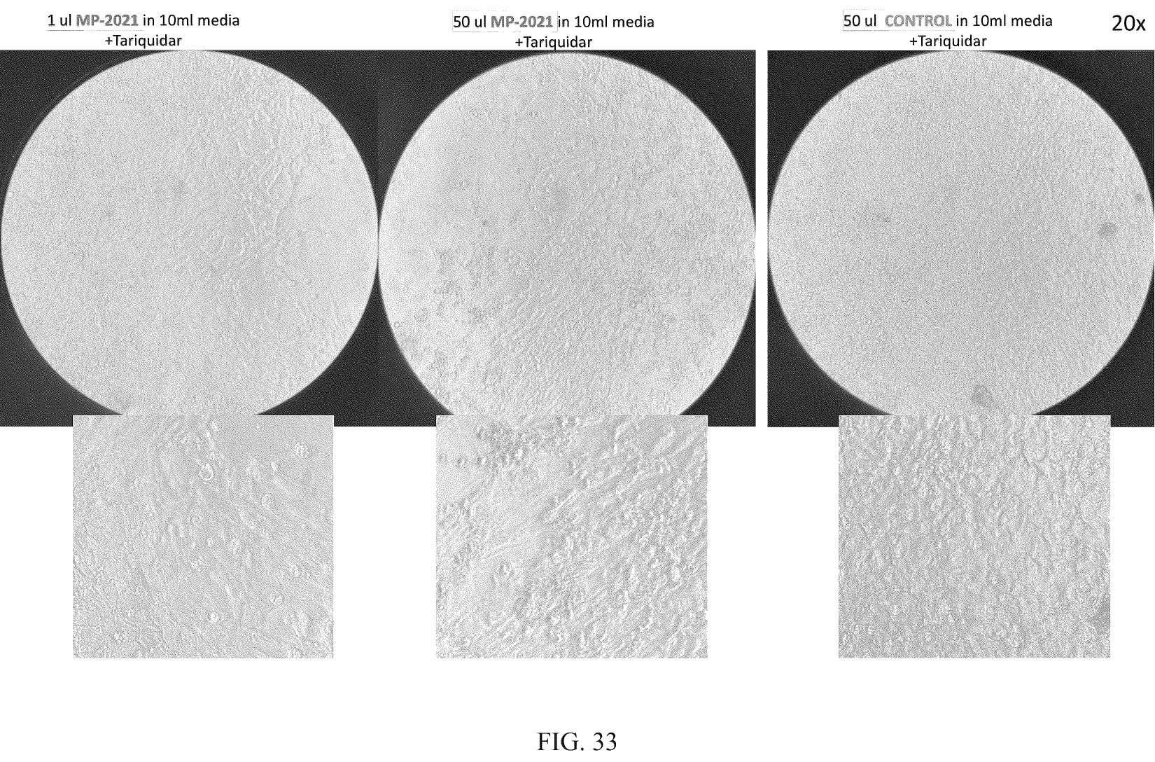

FIG. 33 shows the progress of one experiment for testing one embodiment of MetPhyte and Tariquidar with different amounts of MetPhyte.

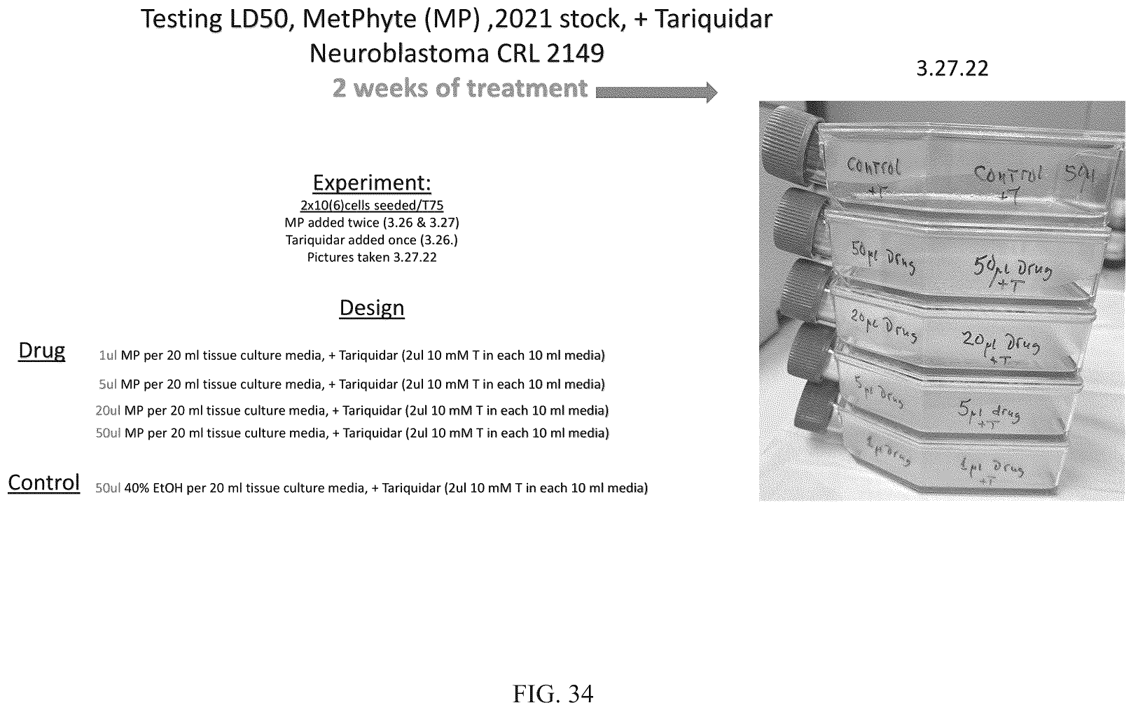

FIG. 34 shows an outline for an experiment for testing one embodiment of MetPhyte and Tariquidar.

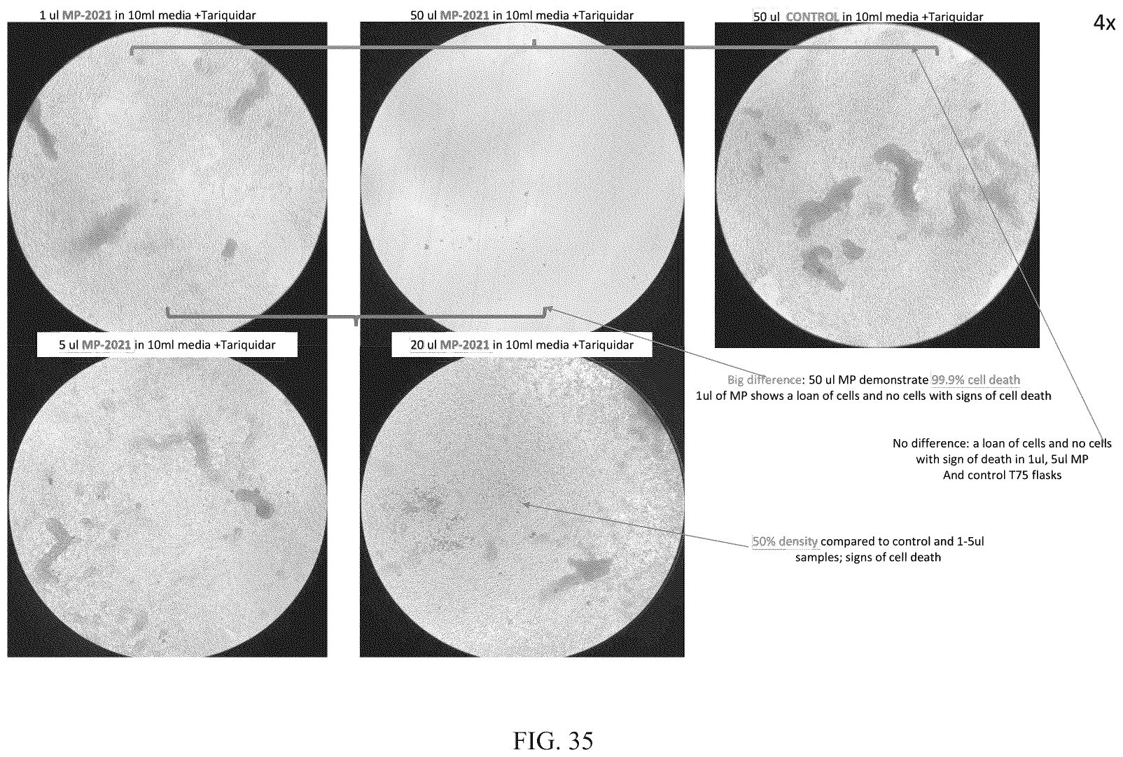

FIG. 35 shows the progress of one experiment for testing one embodiment of MetPhyte and Tariquidar with different amounts of MetPhyte.

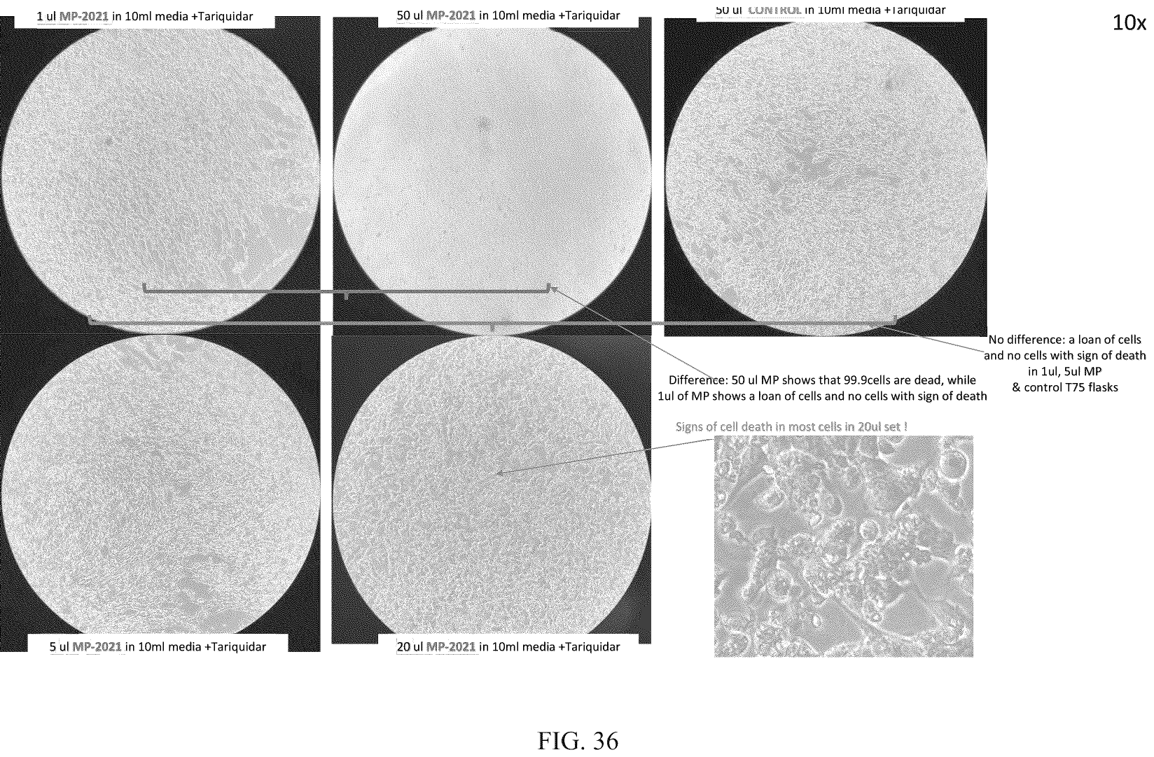

FIG. 36 shows the progress of one experiment for testing one embodiment of MetPhyte and Tariquidar with different amounts of MetPhyte.

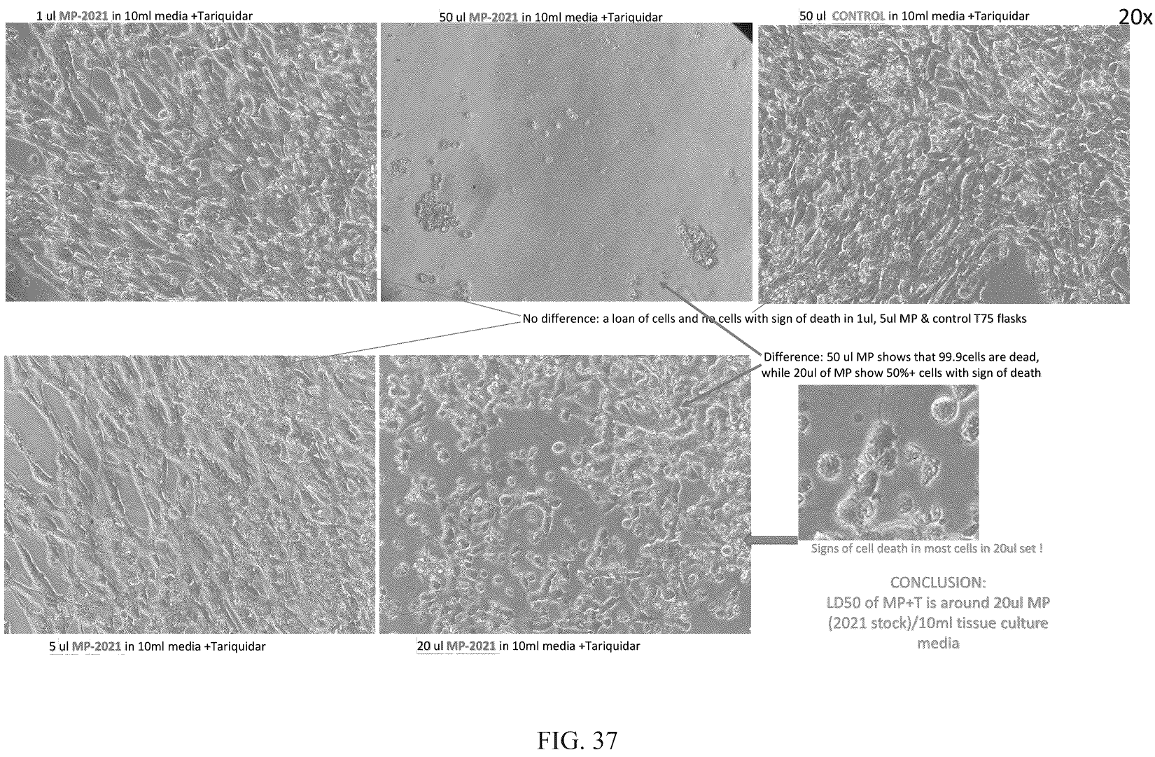

FIG. 37 shows the progress of one experiment for testing one embodiment of MetPhyte and Tariquidar with different amounts of MetPhyte.

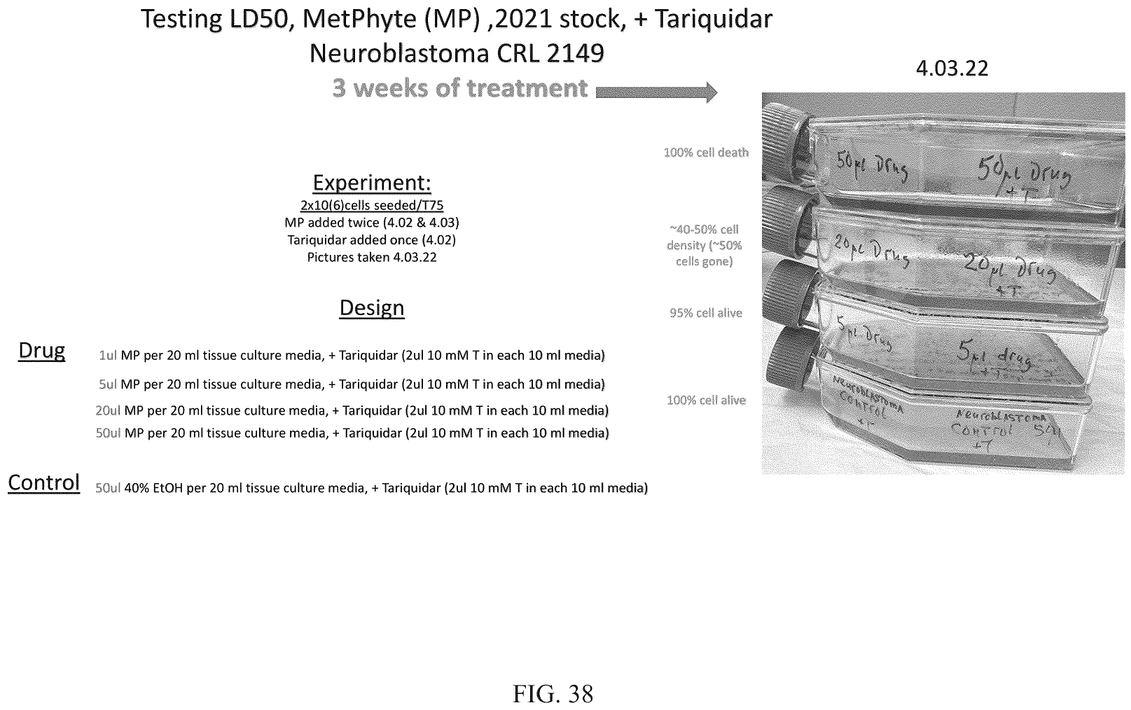

FIG. 38 shows an outline for an experiment for testing one embodiment of MetPhyte and Tariquidar.

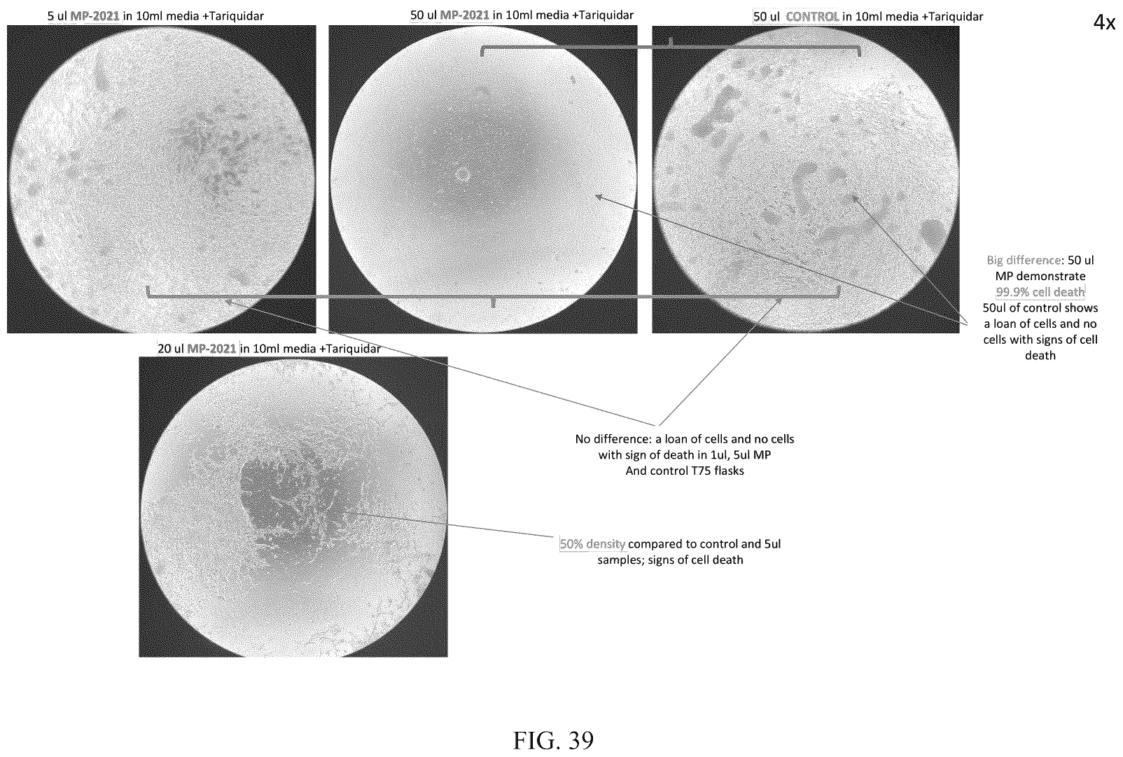

FIG. 39 shows the progress of one experiment for testing one embodiment of MetPhyte and Tariquidar with different amounts of MetPhyte.

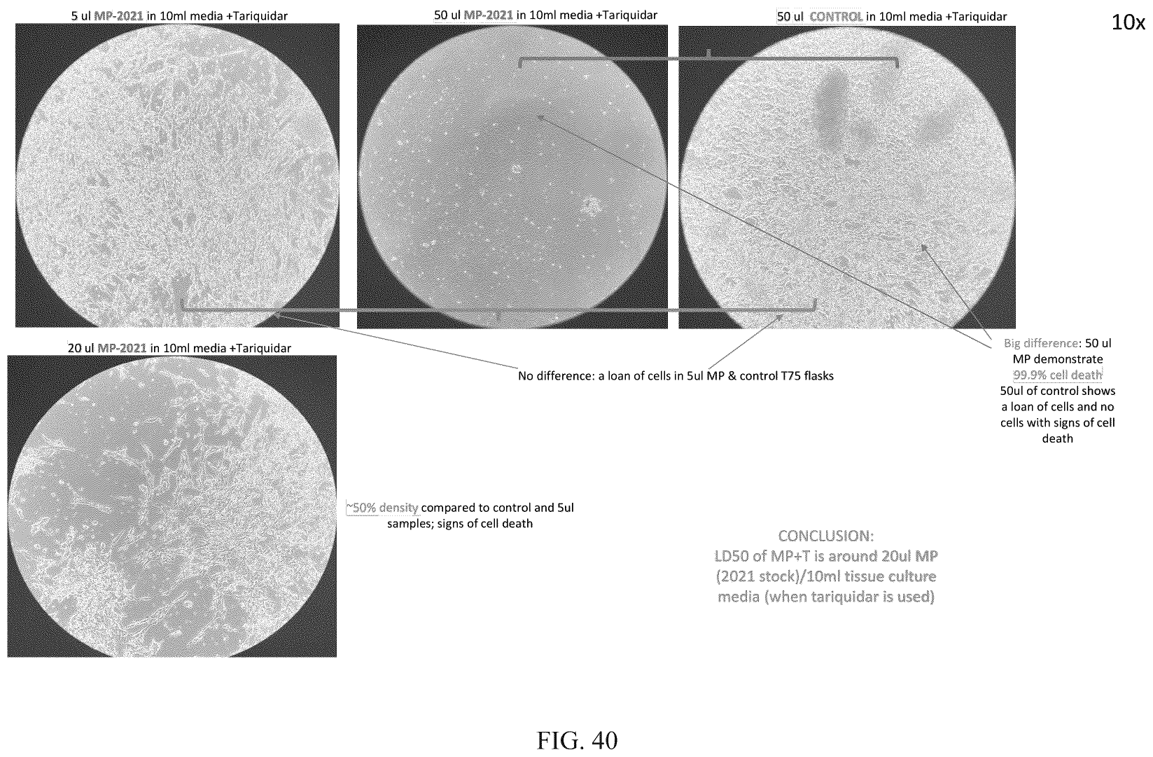

FIG. 40 shows the progress of one experiment for testing one embodiment of MetPhyte and Tariquidar with different amounts of MetPhyte.

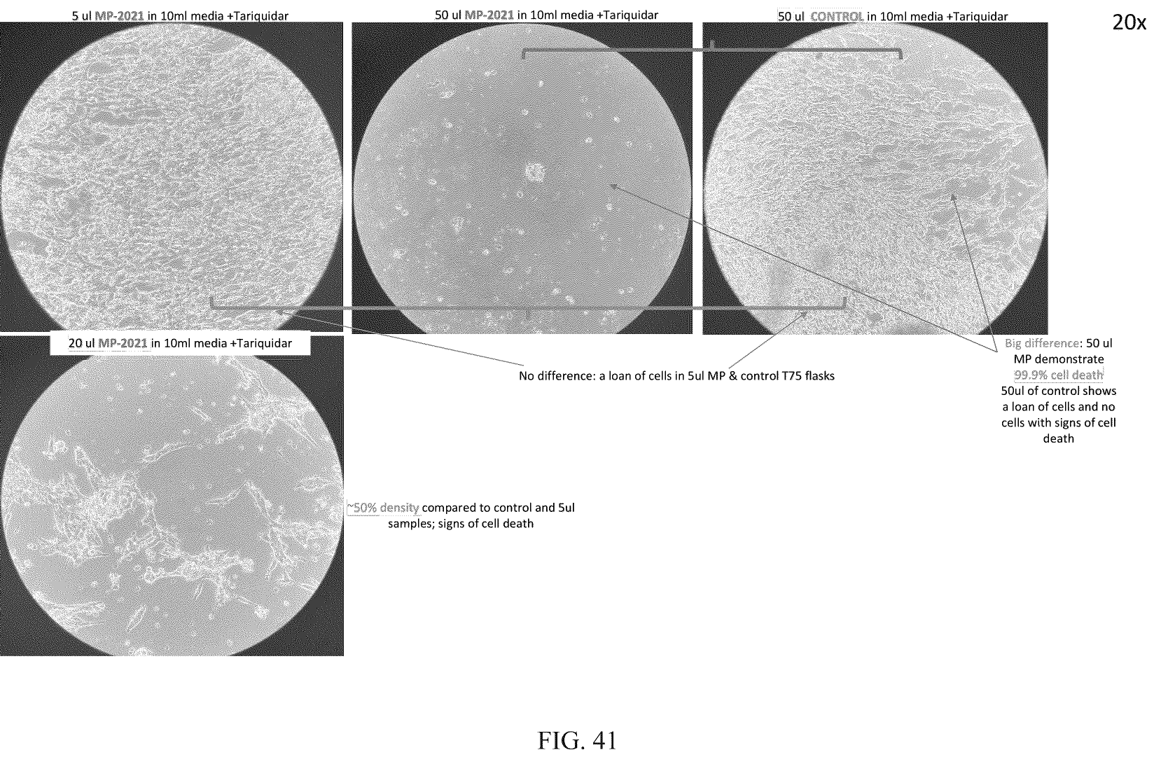

FIG. 41 shows the progress of one experiment for testing one embodiment of MetPhyte and Tariquidar with different amounts of MetPhyte.

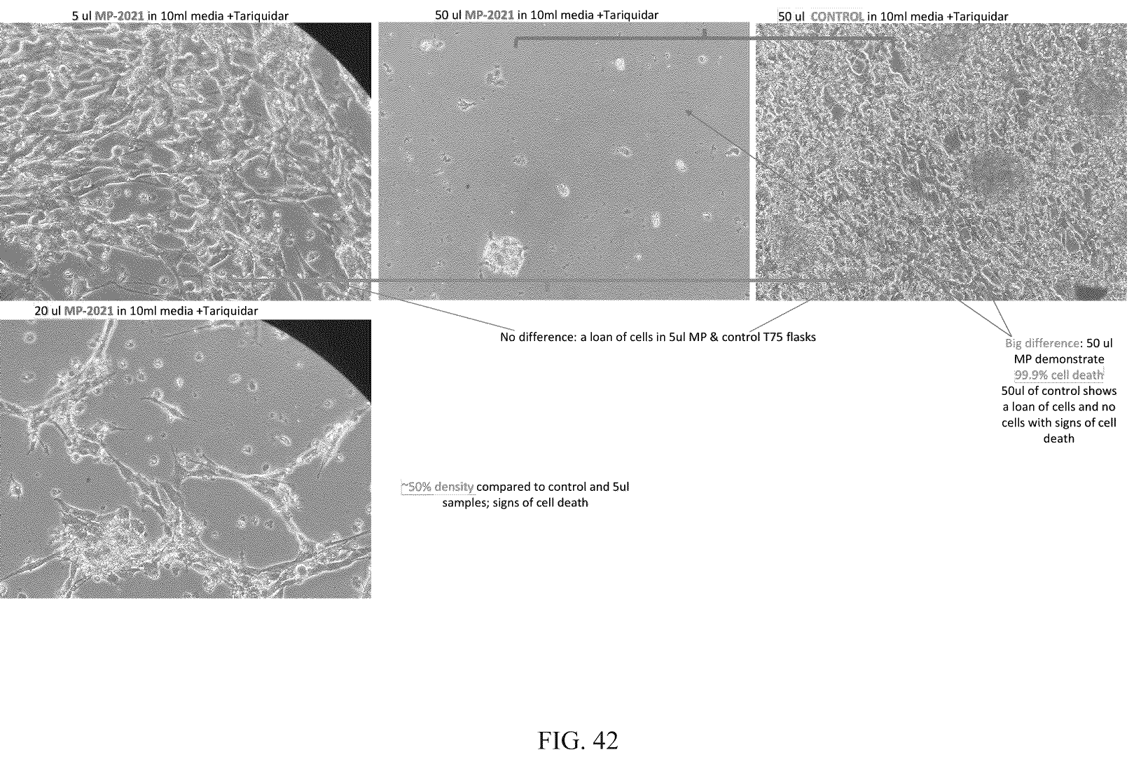

FIG. 42 shows the progress of one experiment for testing one embodiment of MetPhyte and Tariquidar with different amounts of MetPhyte.

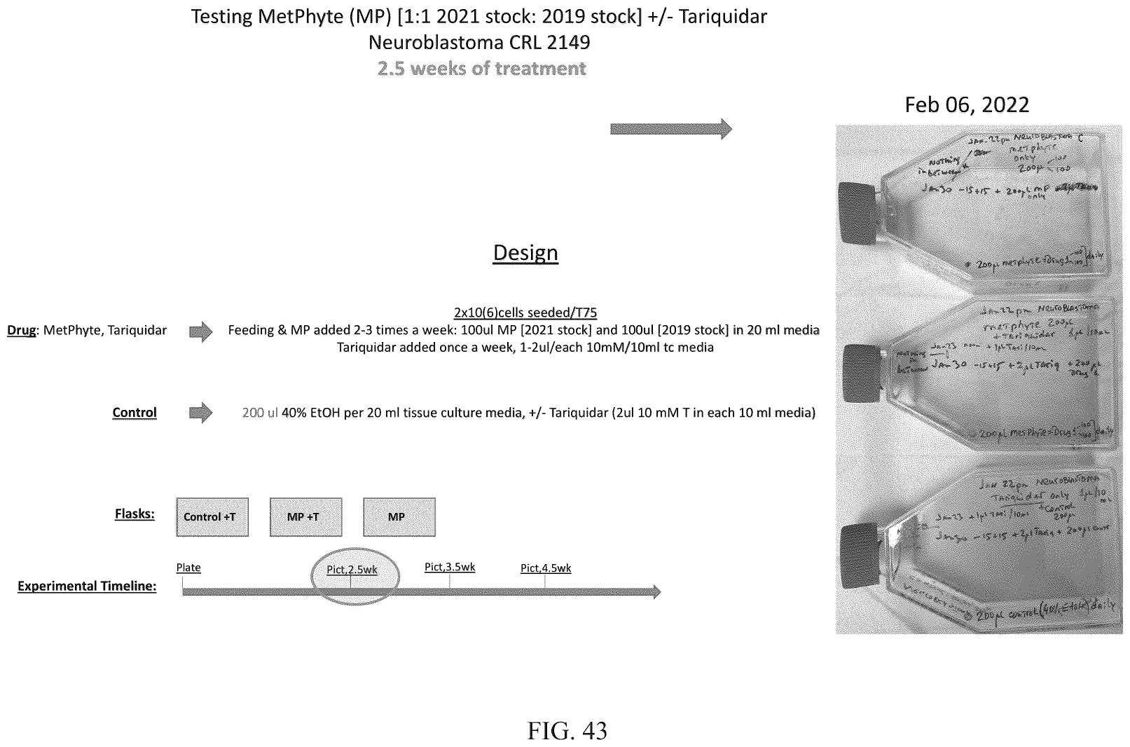

FIG. 43 shows an outline for an experiment for testing one embodiment of MetPhyte and Tariquidar.

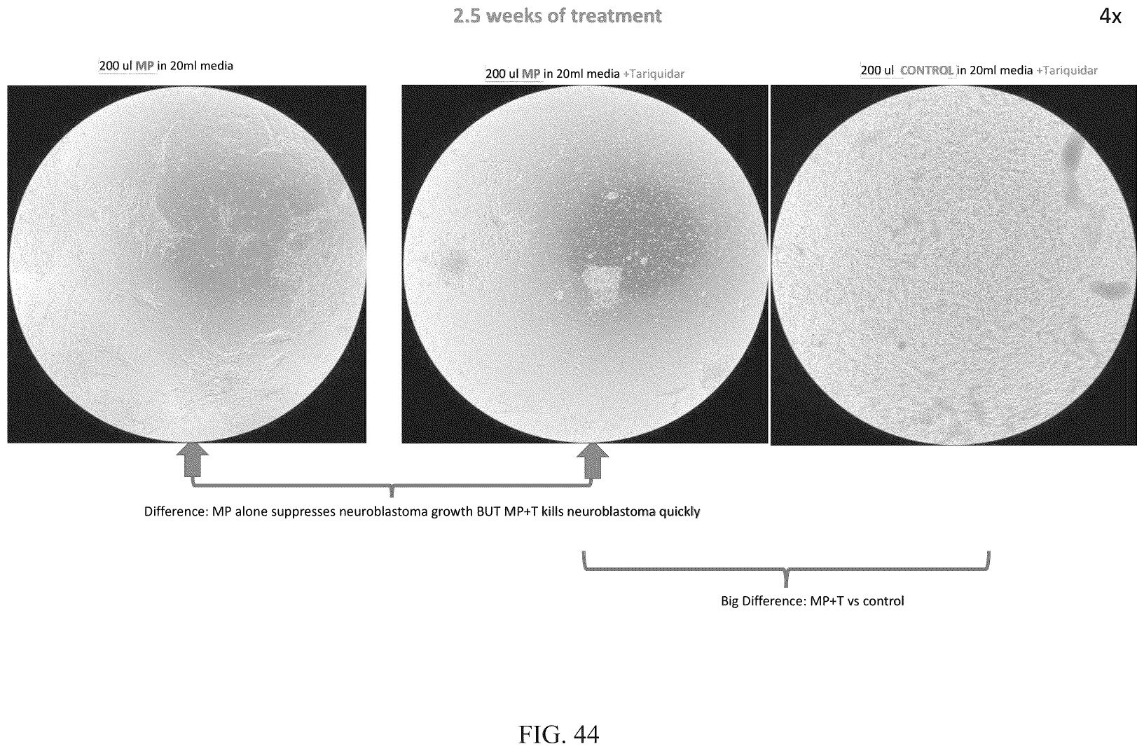

FIG. 44 shows the progress of one experiment for testing one embodiment of MetPhyte and Tariquidar with different amounts of MetPhyte.

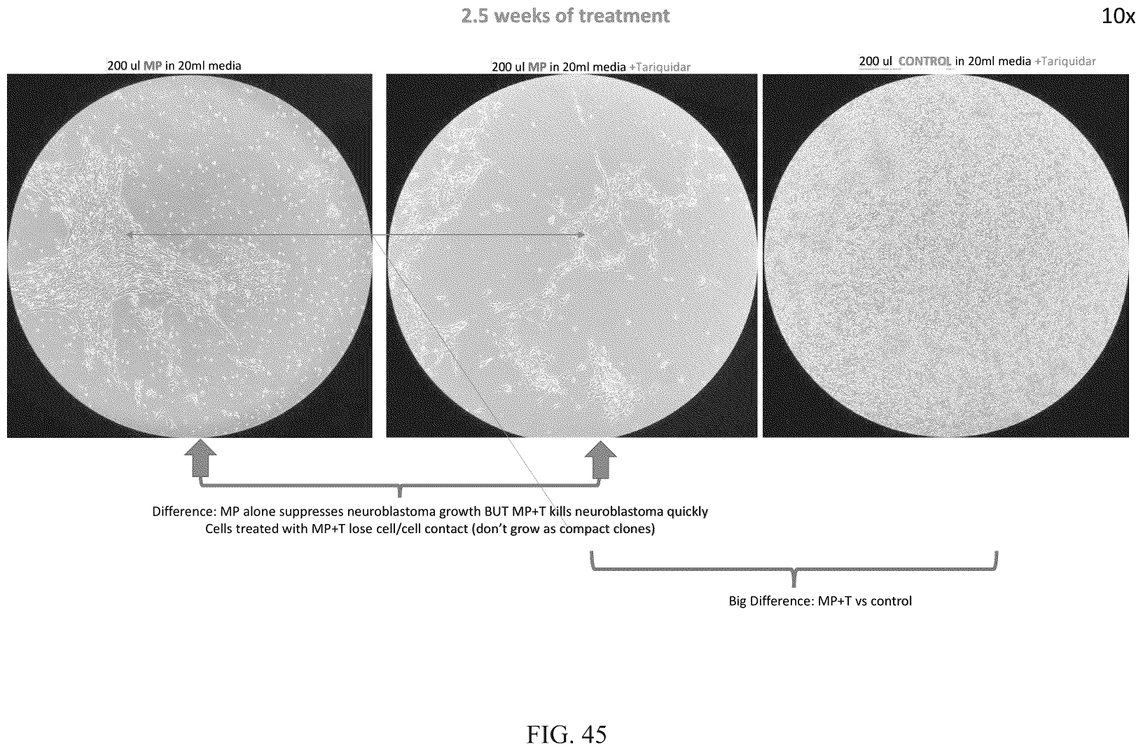

FIG. 45 shows the progress of one experiment for testing one embodiment of MetPhyte and Tariquidar with different amounts of MetPhyte.

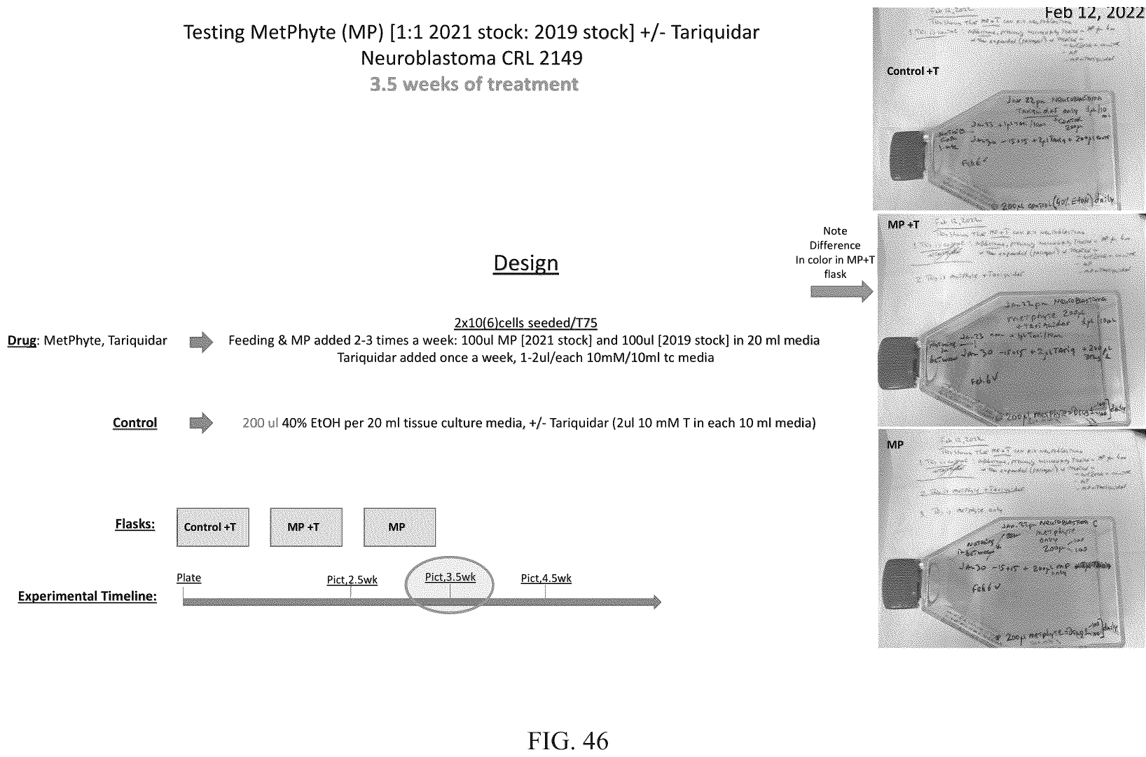

FIG. 46 shows an outline for an experiment for testing one embodiment of MetPhyte and Tariquidar.

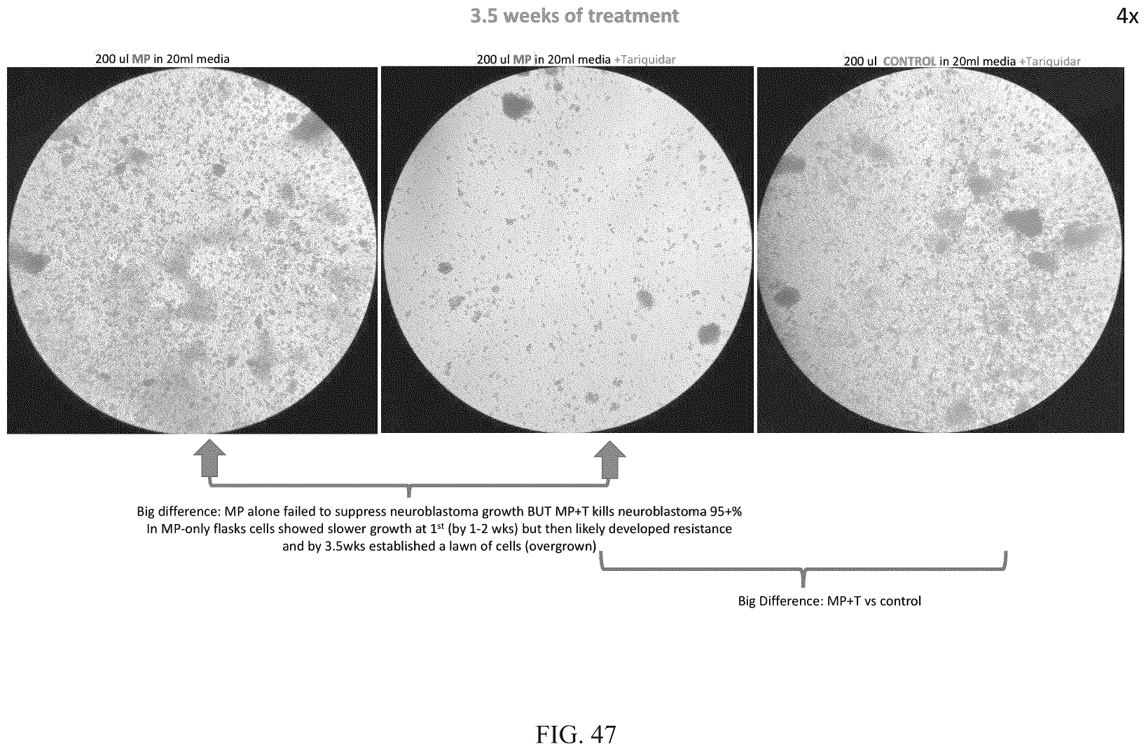

FIG. 47 shows the progress of one experiment for testing one embodiment of MetPhyte and Tariquidar with different amounts of MetPhyte.

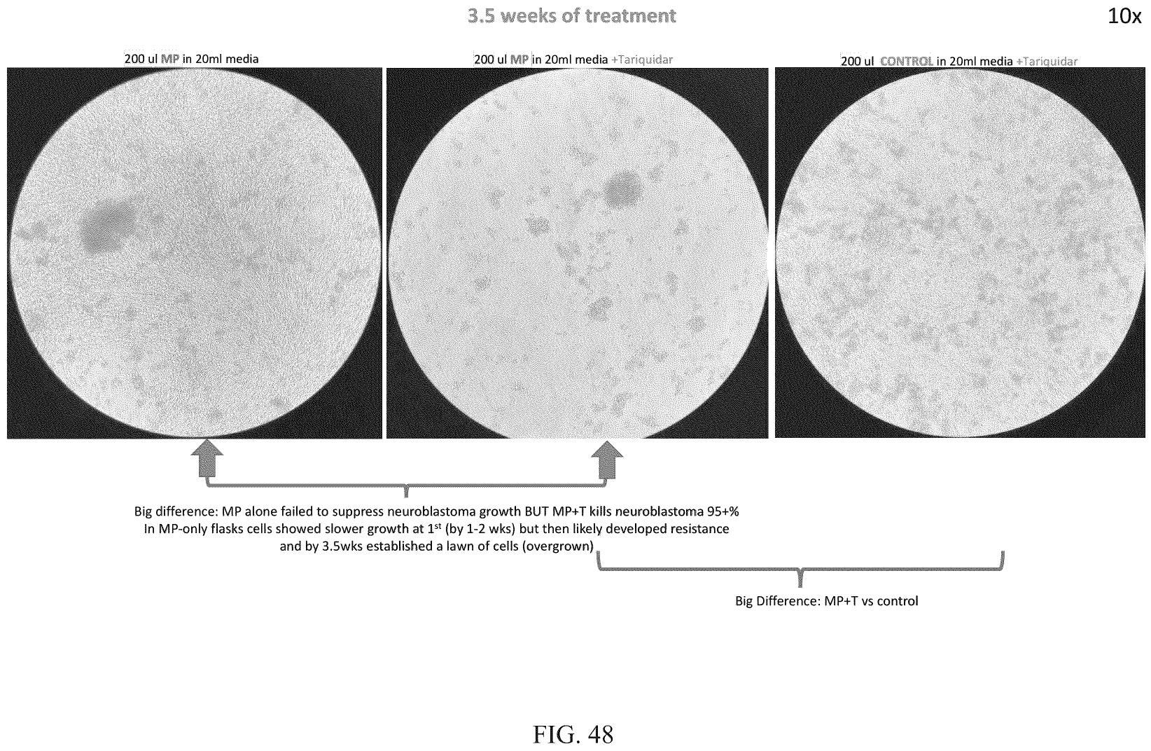

FIG. 48 shows the progress of one experiment for testing one embodiment of MetPhyte and Tariquidar with different amounts of MetPhyte.

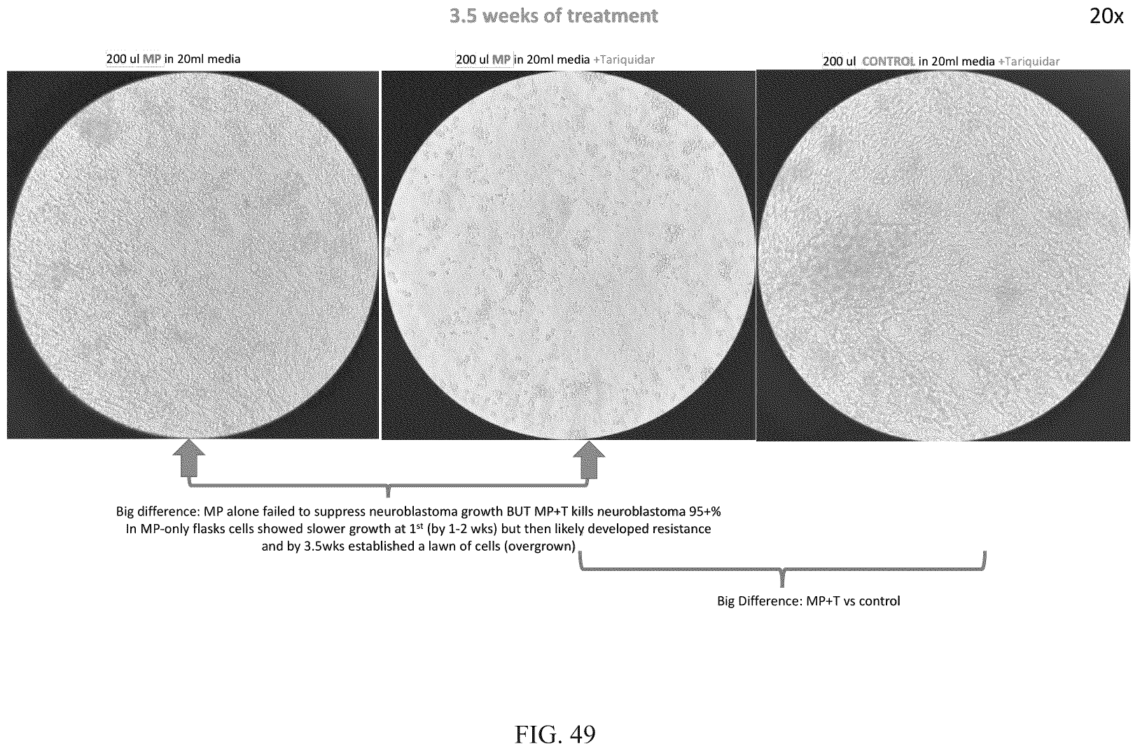

FIG. 49 shows the progress of one experiment for testing one embodiment of MetPhyte and Tariquidar with different amounts of MetPhyte.

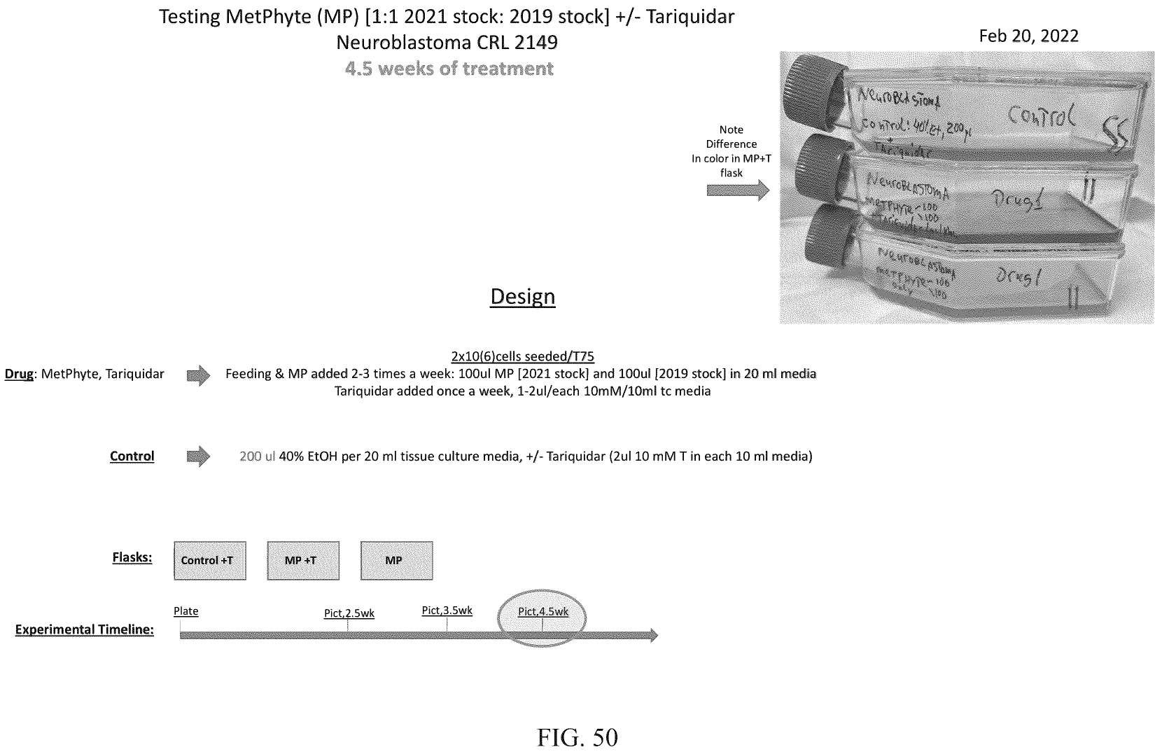

FIG. 50 shows an outline for an experiment for testing one embodiment of MetPhyte and Tariquidar.

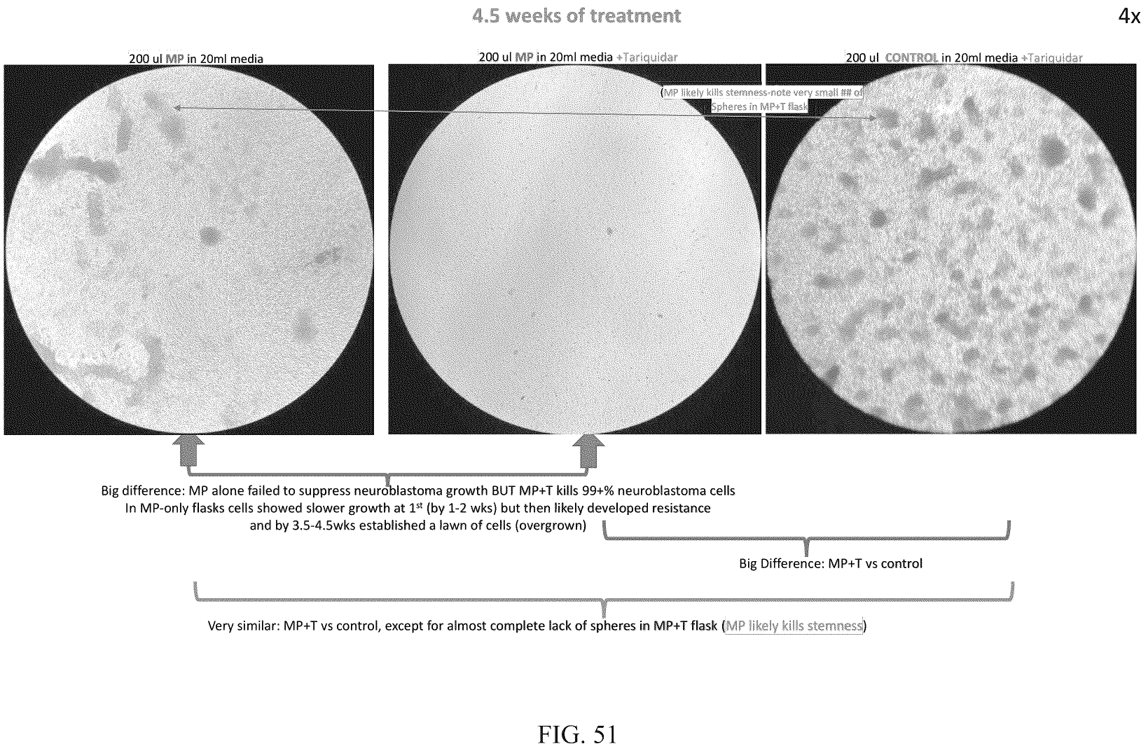

FIG. 51 shows the progress of one experiment for testing one embodiment of MetPhyte and Tariquidar with different amounts of MetPhyte.

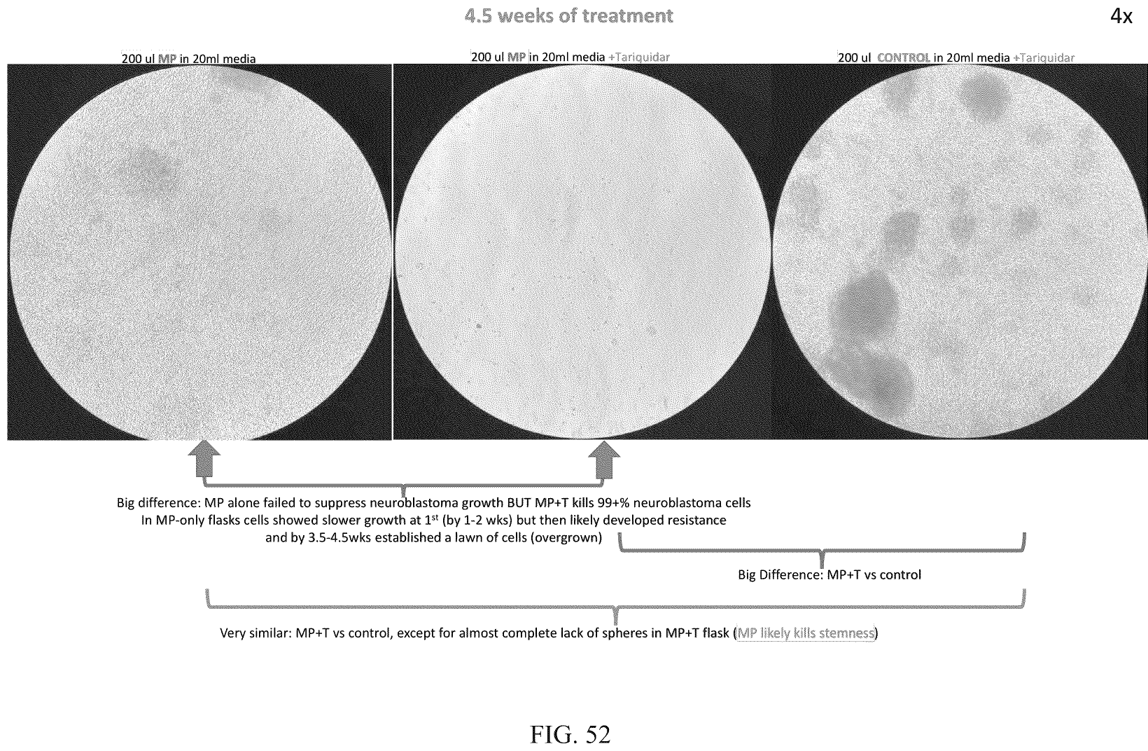

FIG. 52 shows the progress of one experiment for testing one embodiment of MetPhyte and Tariquidar with different amounts of MetPhyte.

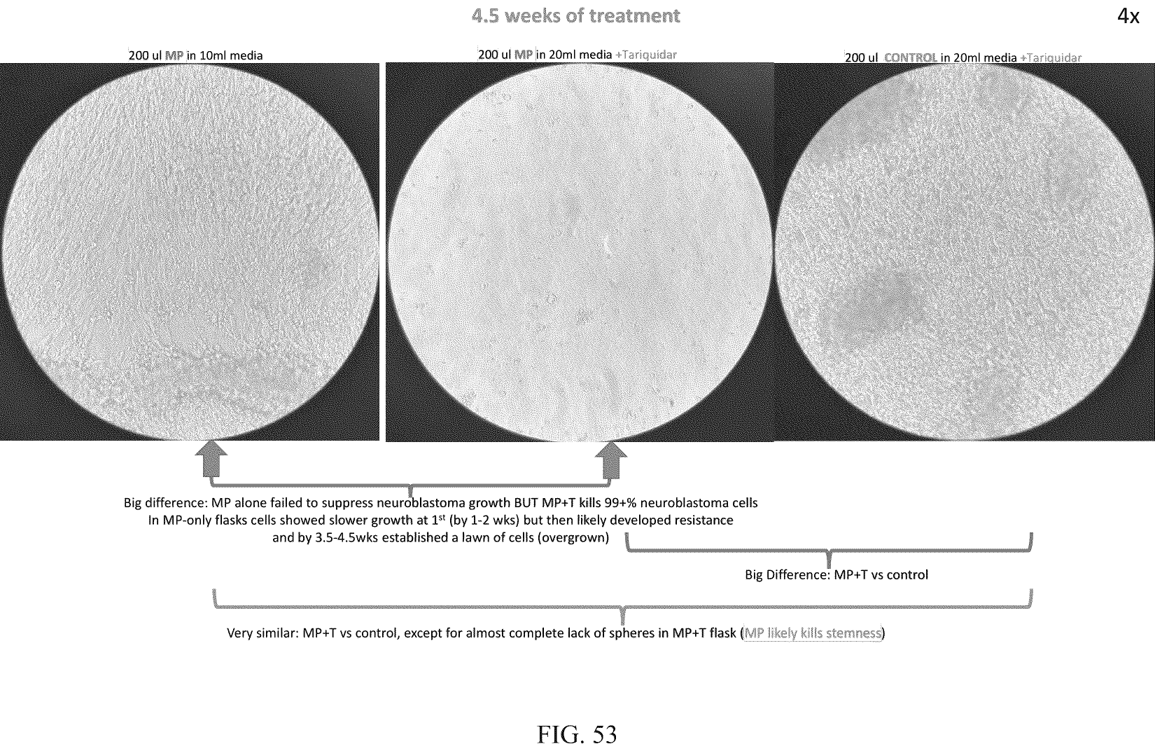

FIG. 53 shows the progress of one experiment for testing one embodiment of MetPhyte and Tariquidar with different amounts of MetPhyte.

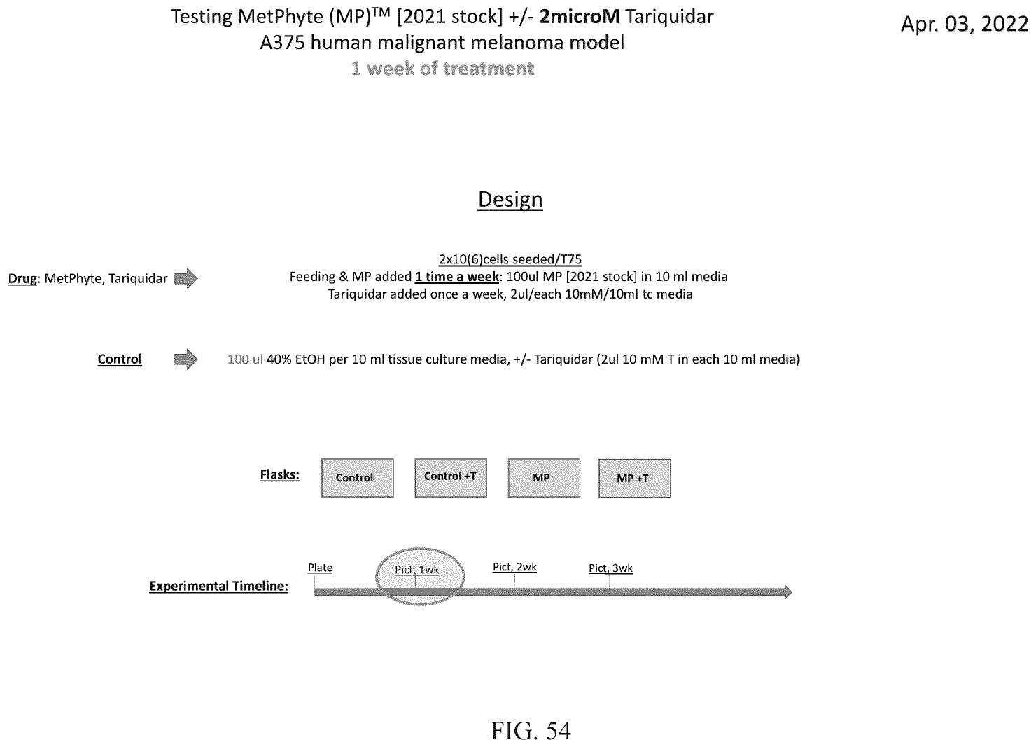

FIG. 54 shows an outline for an experiment for testing one embodiment of MetPhyte and Tariquidar.

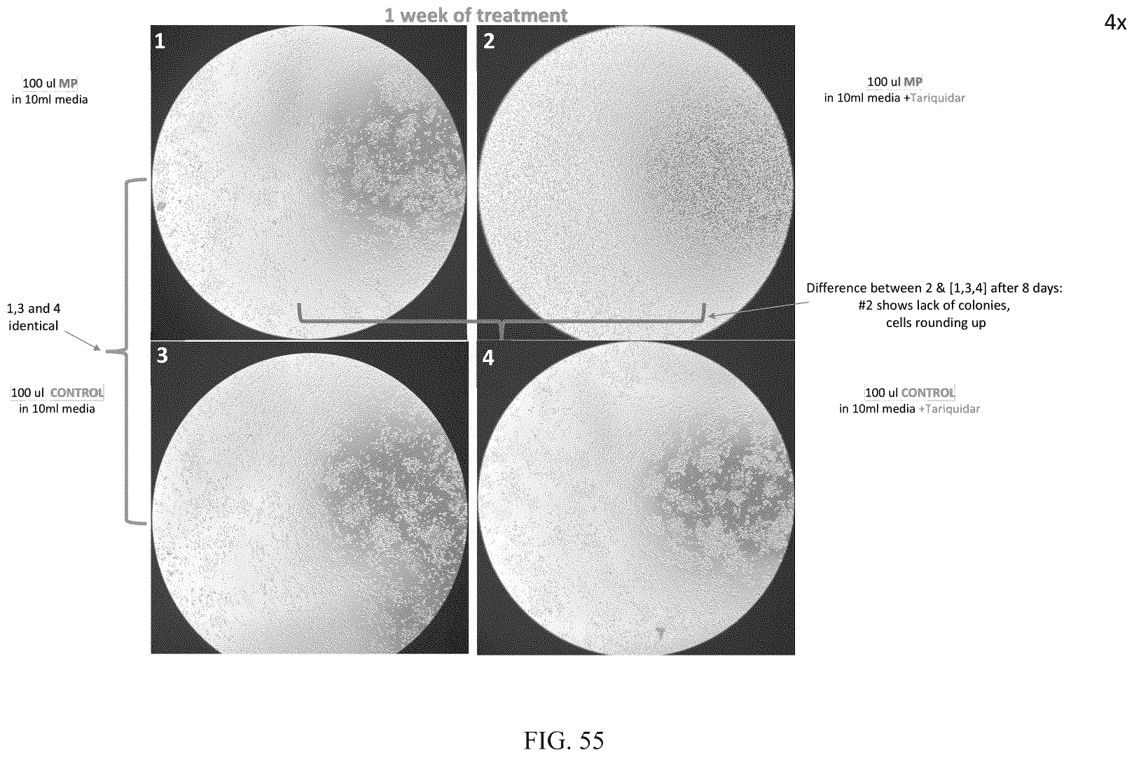

FIG. 55 shows the progress of one experiment for testing one embodiment of MetPhyte and Tariquidar with different amounts of MetPhyte.

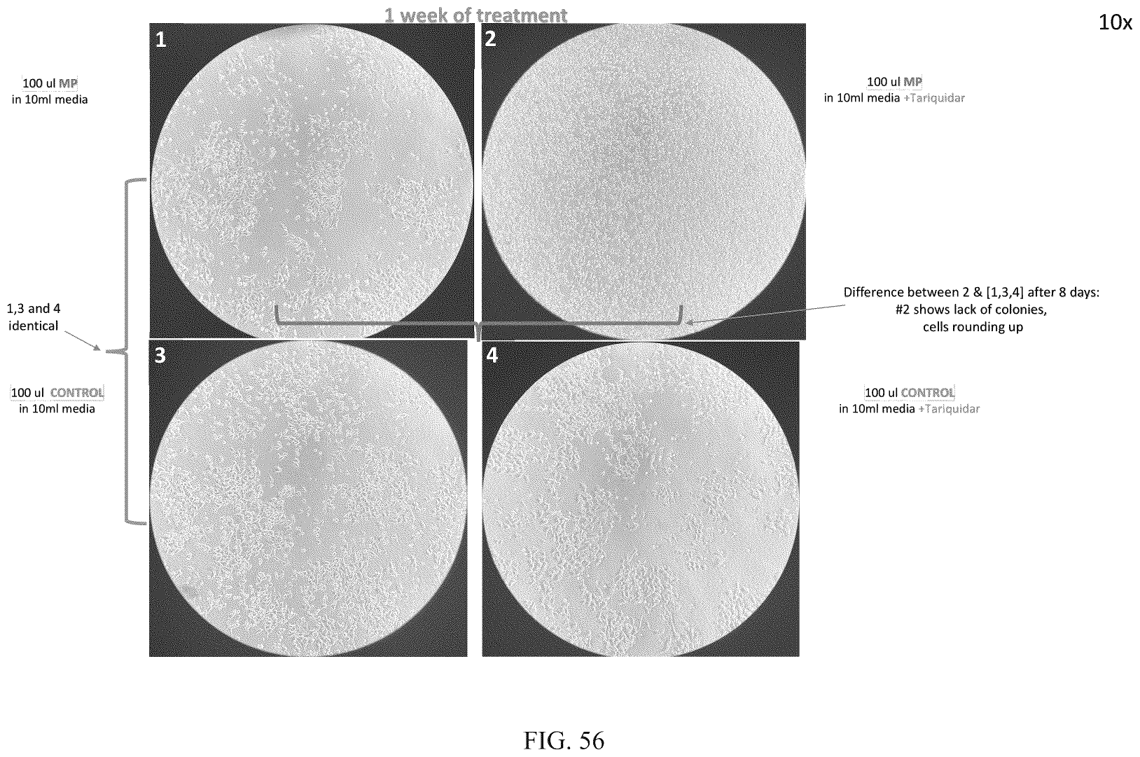

FIG. 56 shows the progress of one experiment for testing one embodiment of MetPhyte and Tariquidar with different amounts of MetPhyte.

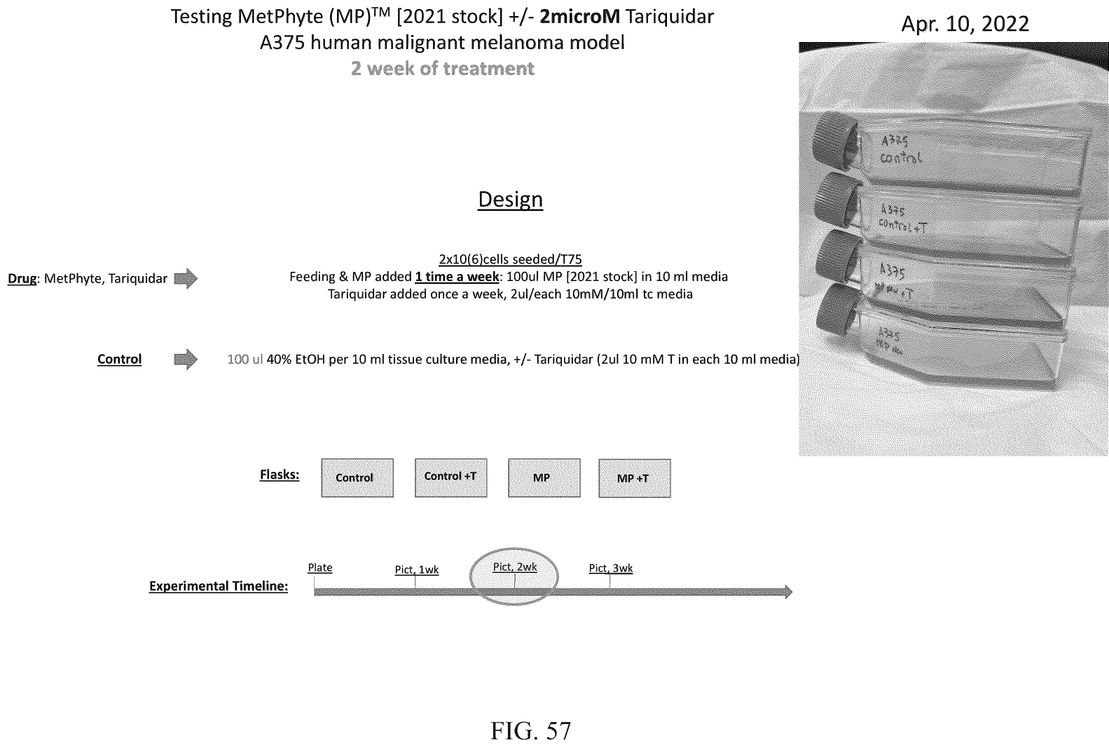

FIG. 57 shows an outline for an experiment for testing one embodiment of MetPhyte and Tariquidar.

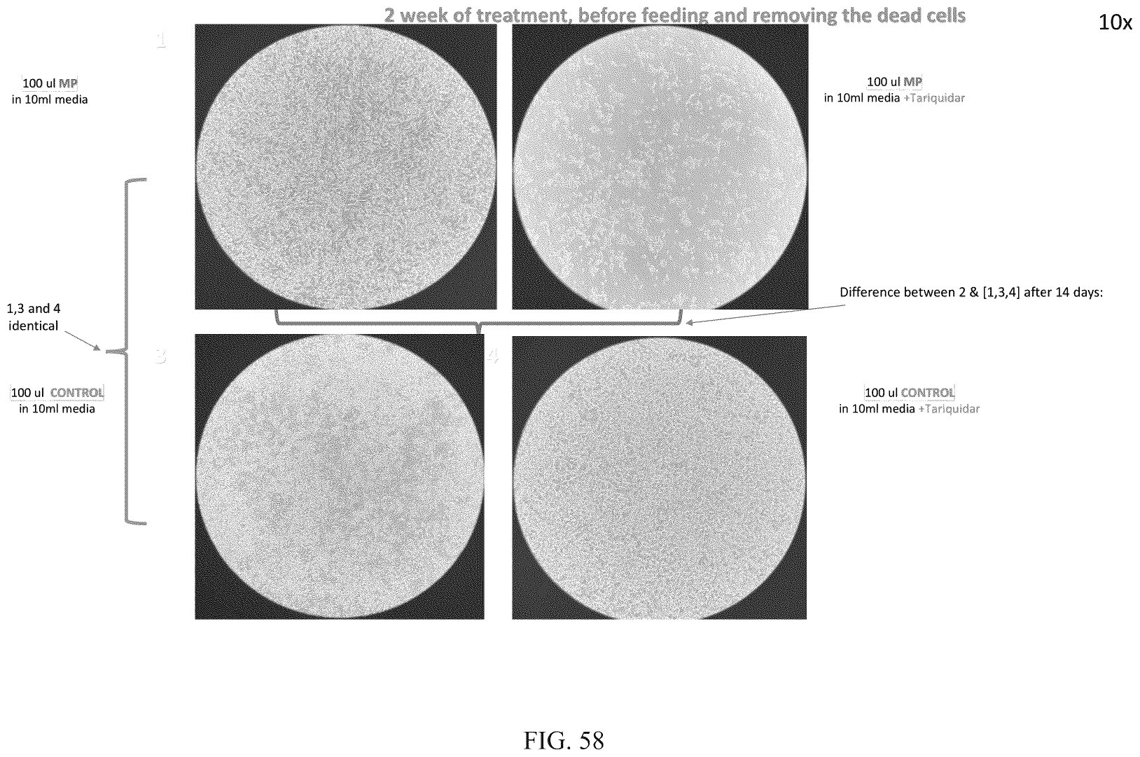

FIG. 58 shows the progress of one experiment for testing one embodiment of MetPhyte and Tariquidar with different amounts of MetPhyte.

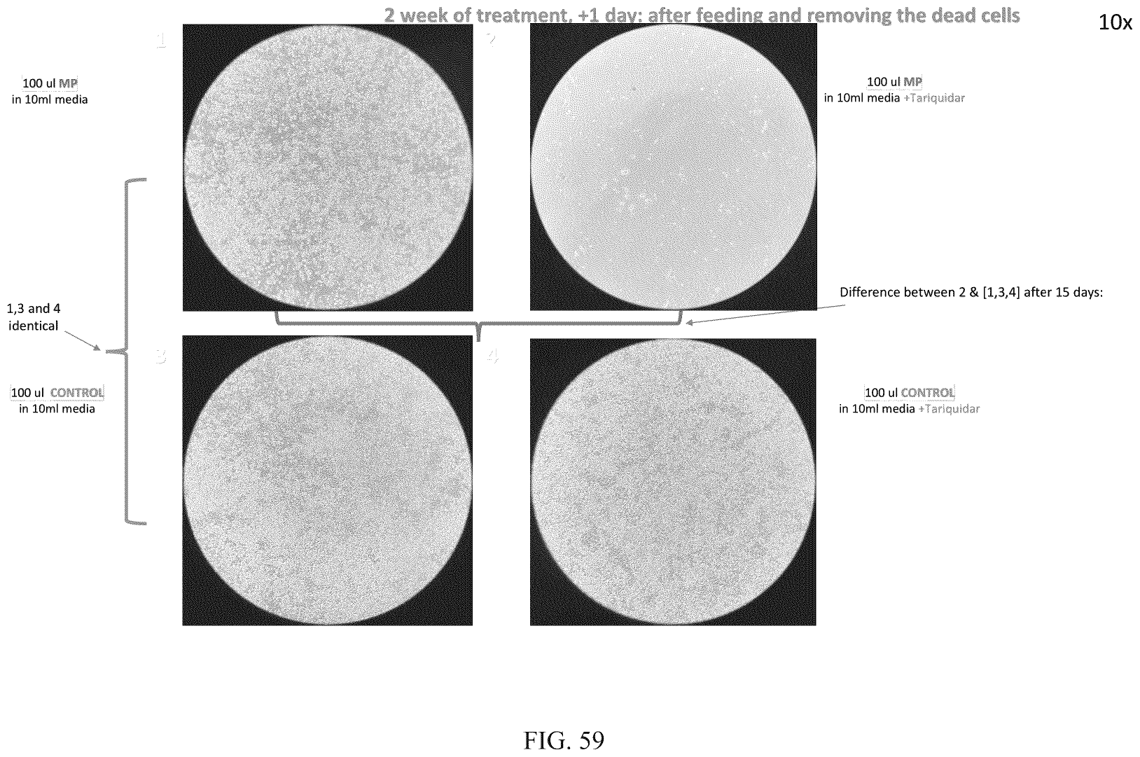

FIG. 59 shows the progress of one experiment for testing one embodiment of MetPhyte and Tariquidar with different amounts of MetPhyte.

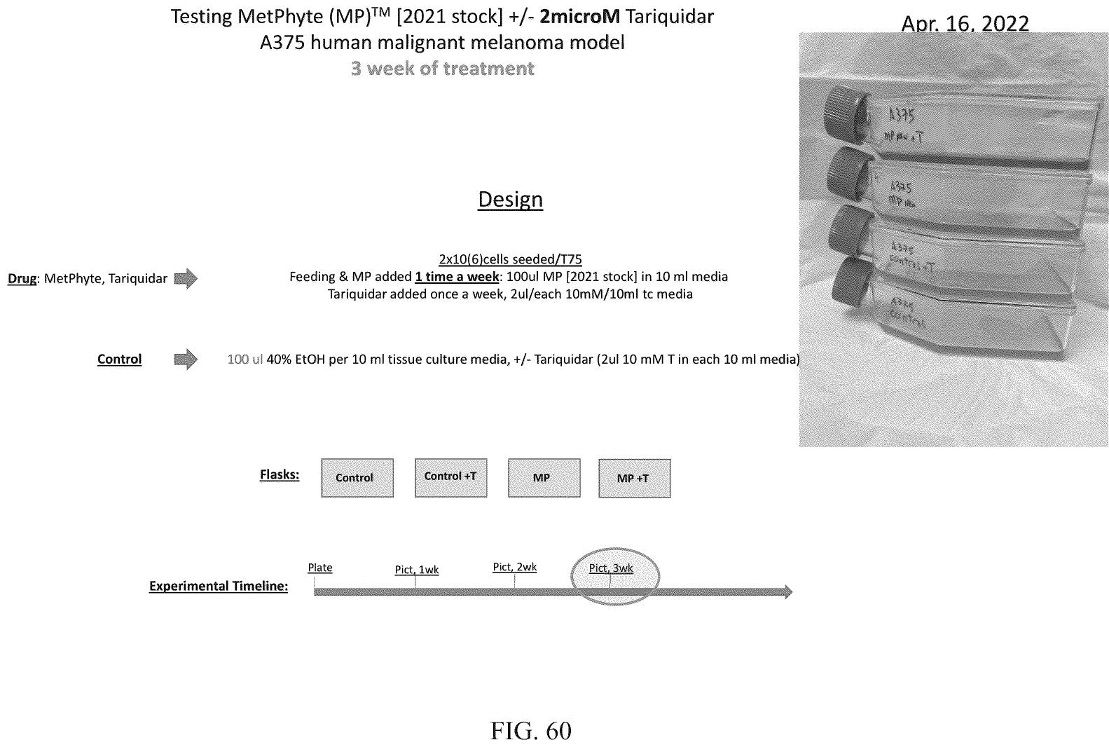

FIG. 60 shows an outline for an experiment for testing one embodiment of MetPhyte and Tariquidar.

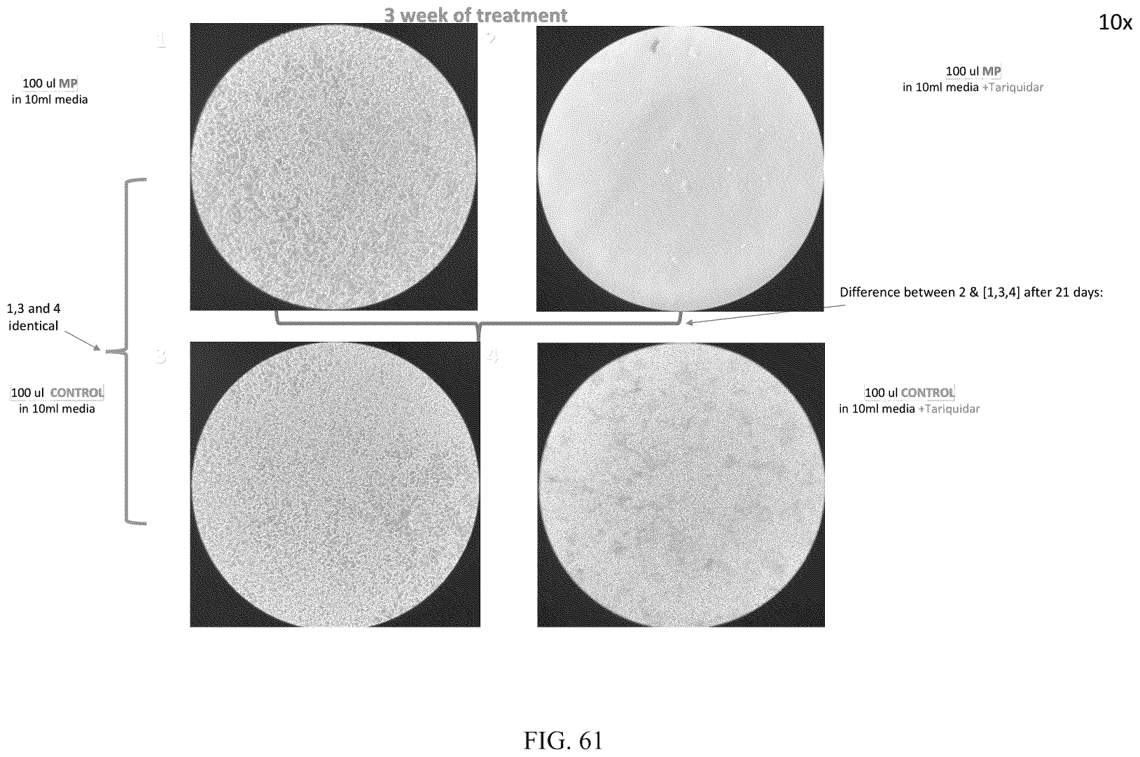

FIG. 61 shows the progress of one experiment for testing one embodiment of MetPhyte and Tariquidar with different amounts of MetPhyte.

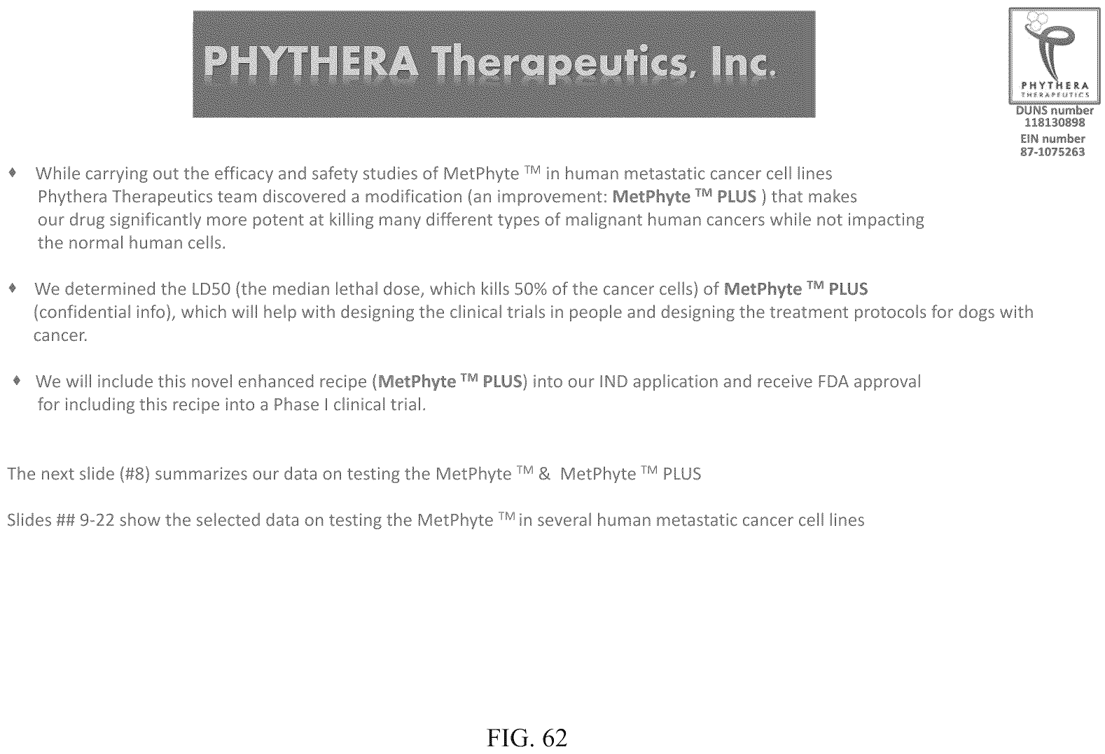

FIG. 62 provides an overview of the disclosure of the present application, including motivation to application thereof.

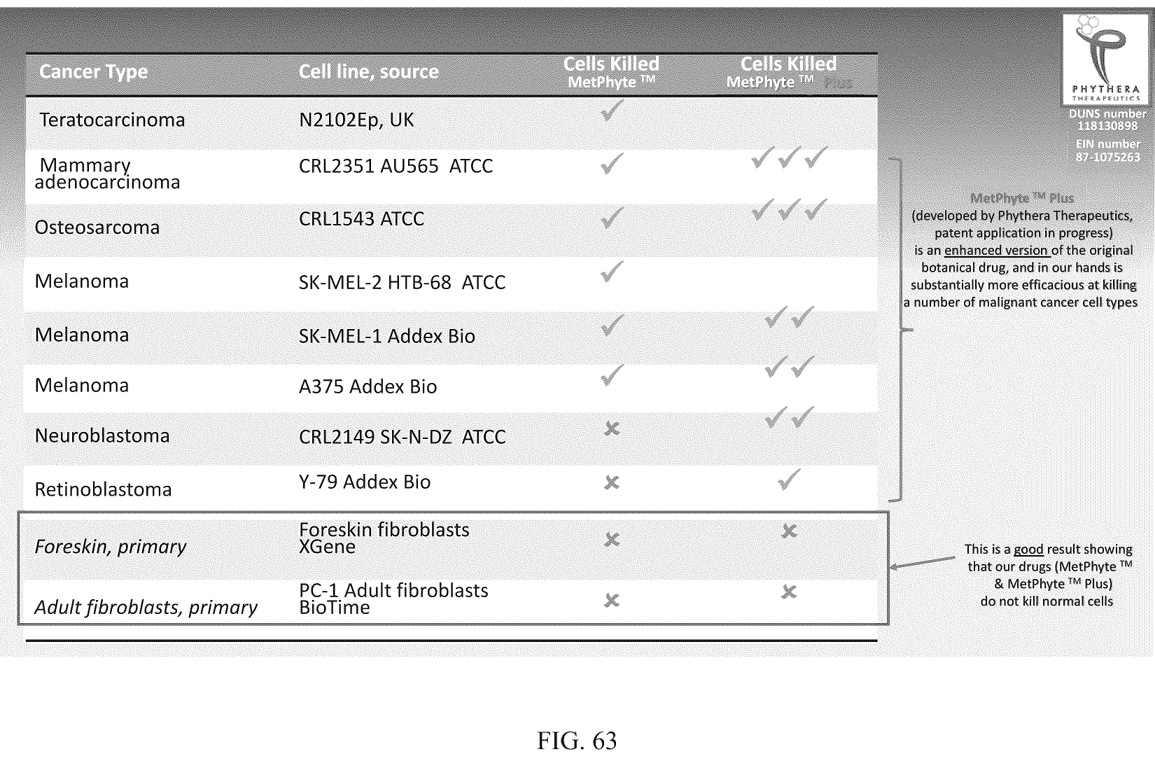

FIG. 63 is a summary of the efficacy of MetPhyte and MetPhyte PLUS on different cell lines.

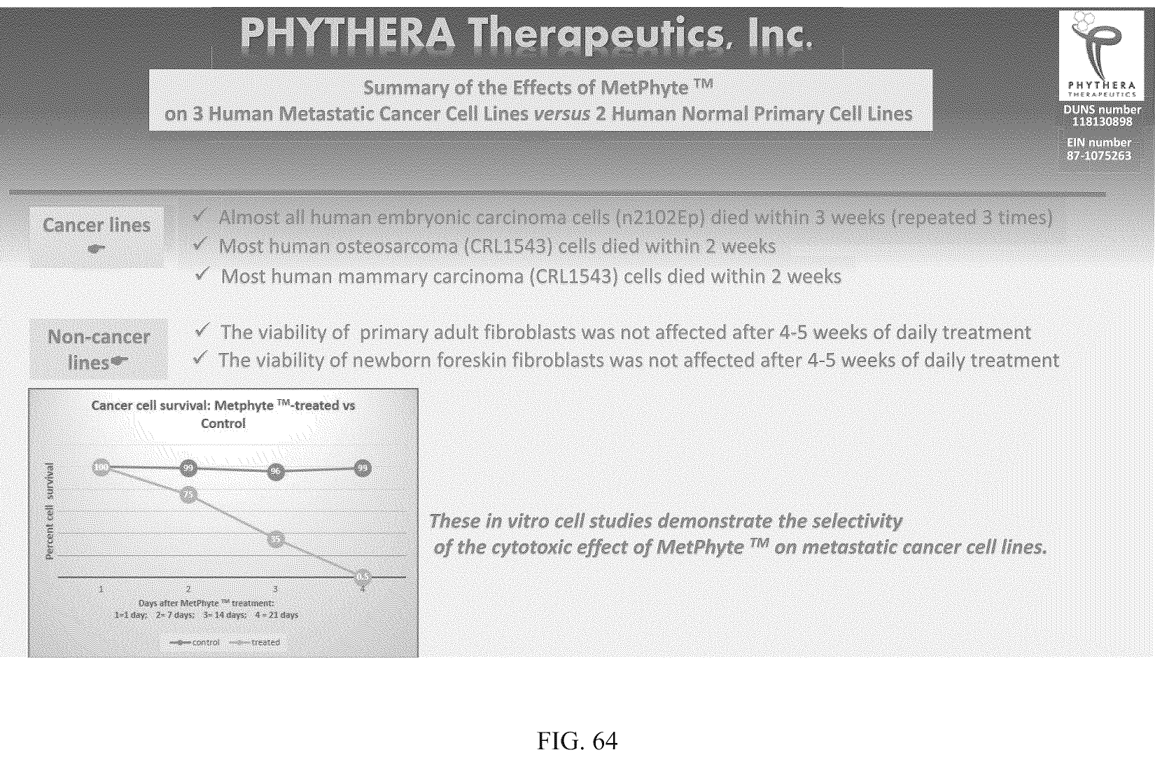

FIG. 64 shows a summary of the efficacy of MetPhyte.

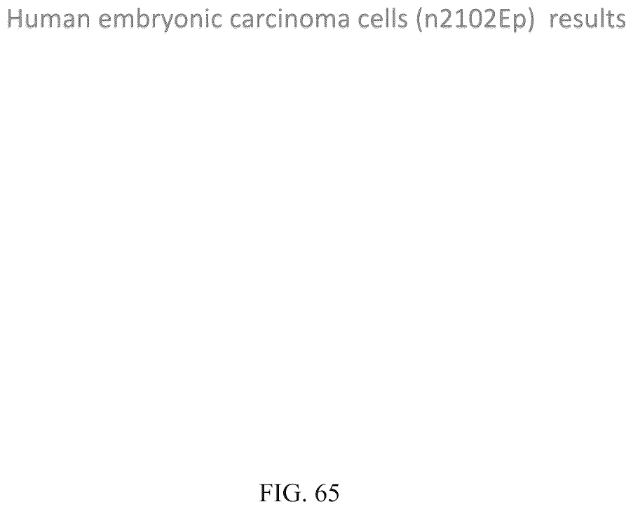

FIG. 65 is a title page for subsequent figures showing efficacy of MetPhyte on human embryonic carcinoma cells (n2102Ep).

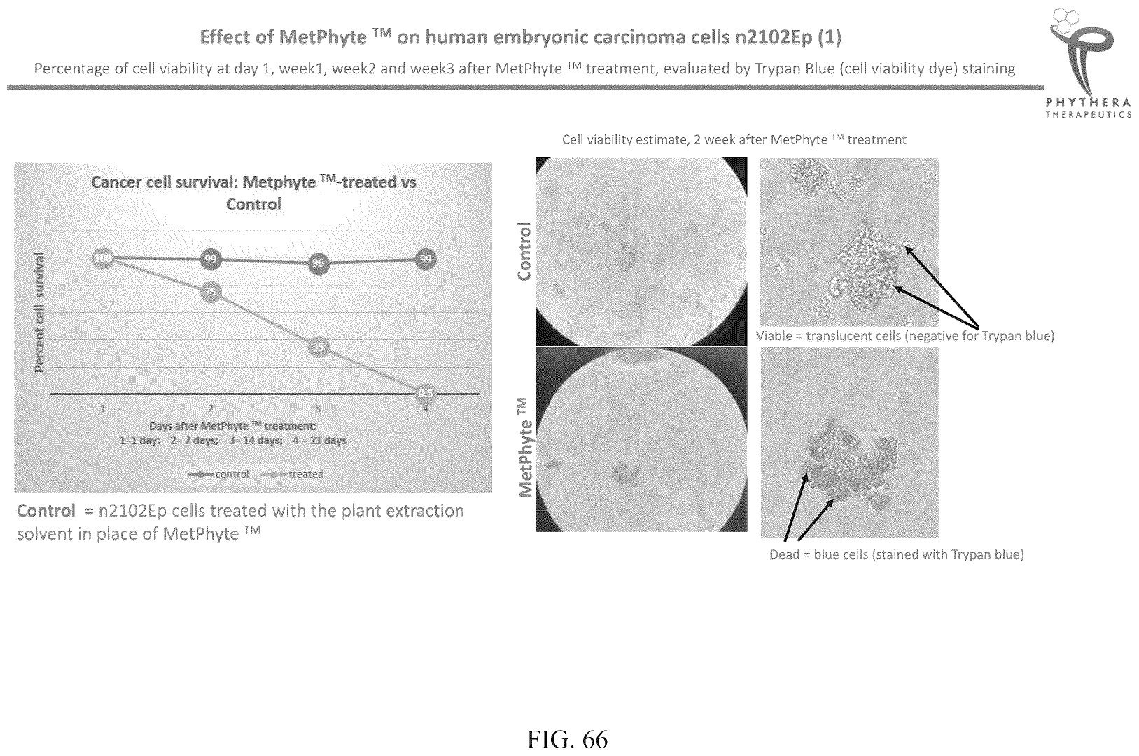

FIG. 66 shows the progress of one experiment for testing one embodiment of MetPhyte on human embryonic carcinoma cells n2102Ep.

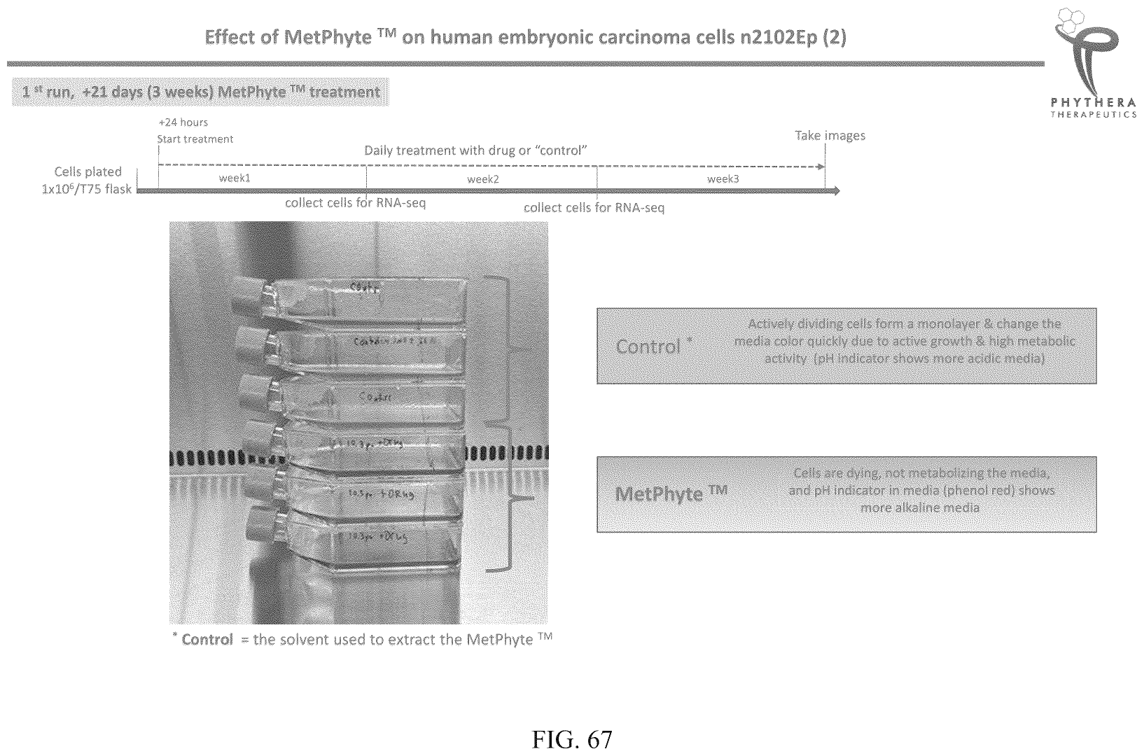

FIG. 67 shows the progress of one experiment for testing one embodiment of MetPhyte on human embryonic carcinoma cells n2102Ep.

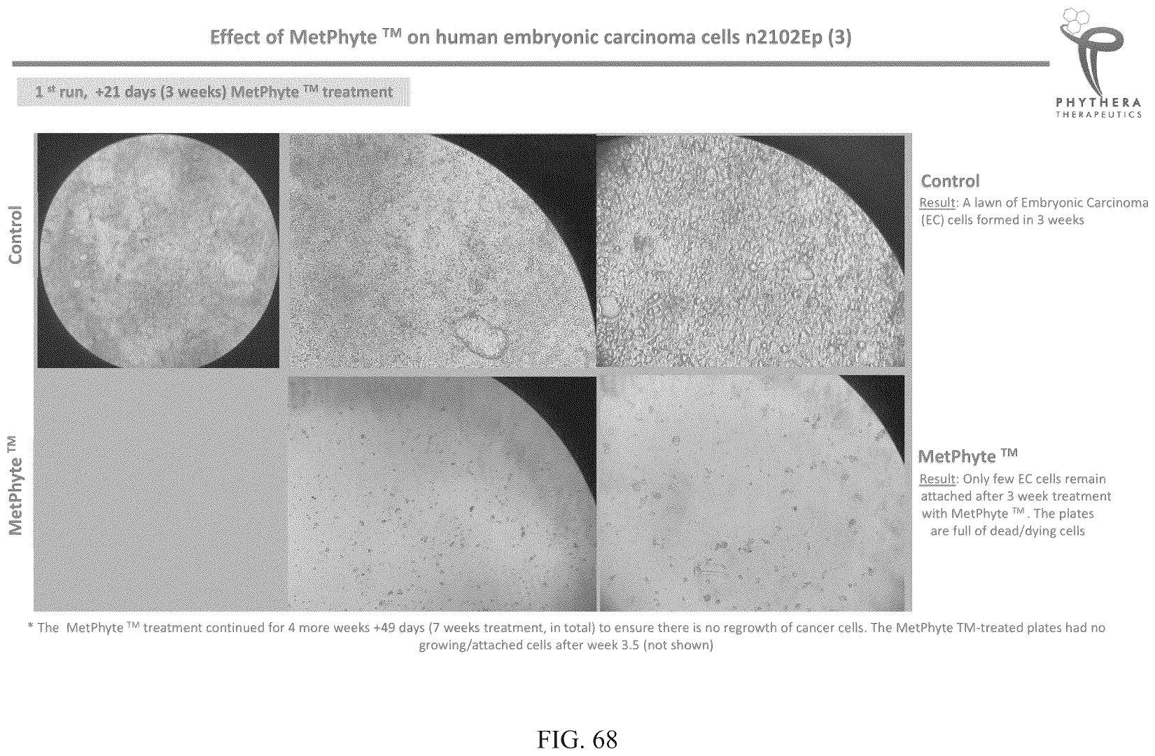

FIG. 68 shows the progress of one experiment for testing one embodiment of MetPhyte on human embryonic carcinoma cells n2102Ep.

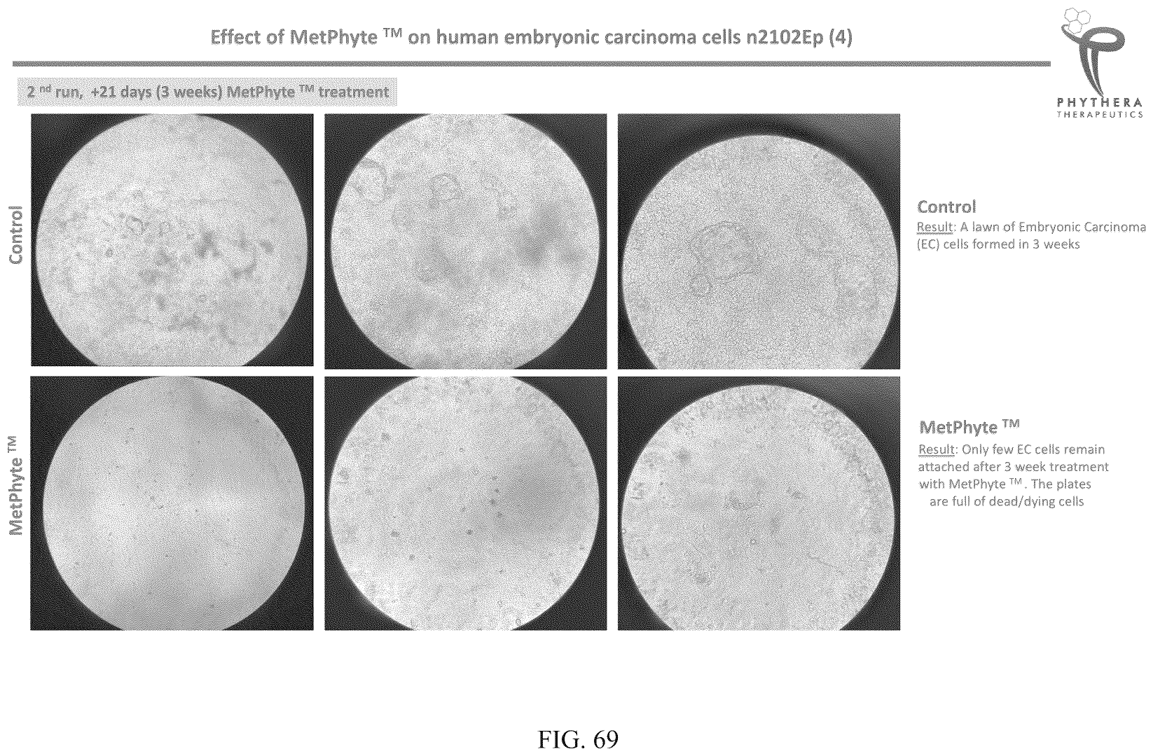

FIG. 69 shows the progress of one experiment for testing one embodiment of MetPhyte on human embryonic carcinoma cells n2102Ep.

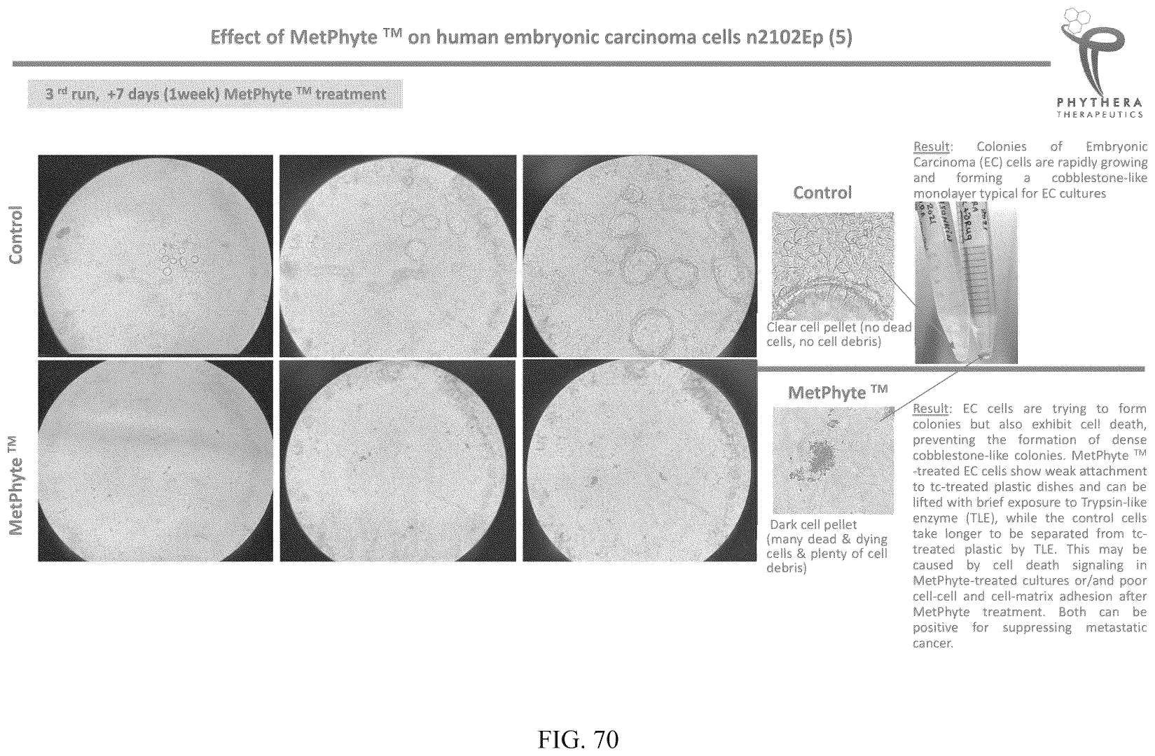

FIG. 70 shows the progress of one experiment for testing one embodiment of MetPhyte on human embryonic carcinoma cells n2102Ep.

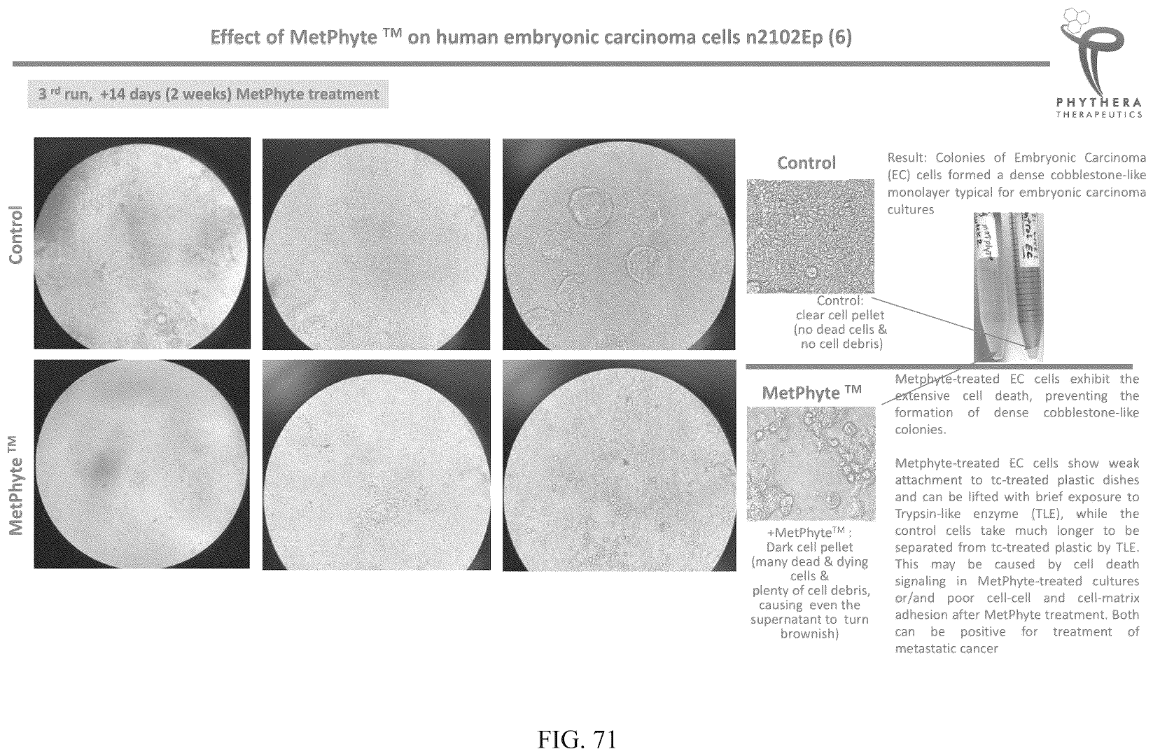

FIG. 71 shows the progress of one experiment for testing one embodiment of MetPhyte on human embryonic carcinoma cells n2102Ep.

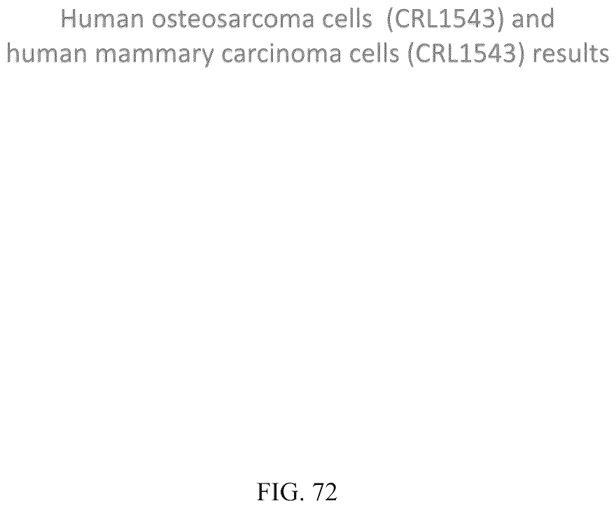

FIG. 72 is a title page for subsequent figures showing efficacy of MetPhyte on human osteosarcoma (CRL1543) and human mammary carcinoma (CRL1543) cells.

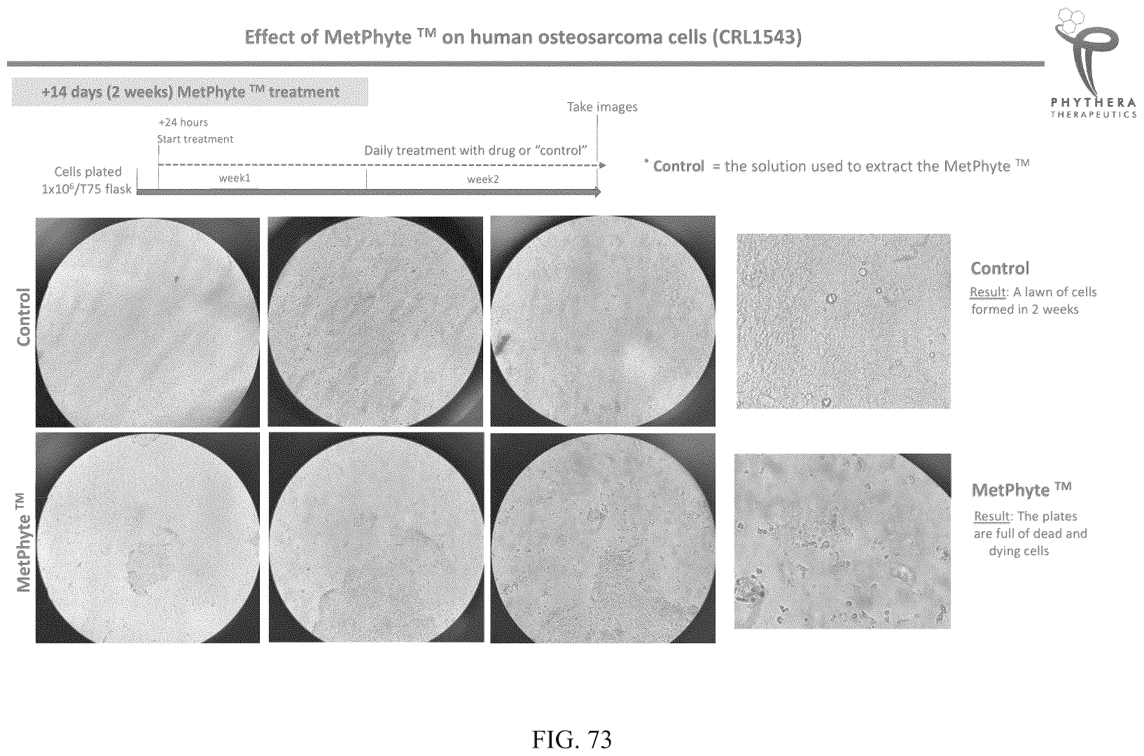

FIG. 73 shows the progress of one experiment for testing one embodiment of MetPhyte on human osteosarcoma cells n2102Ep.

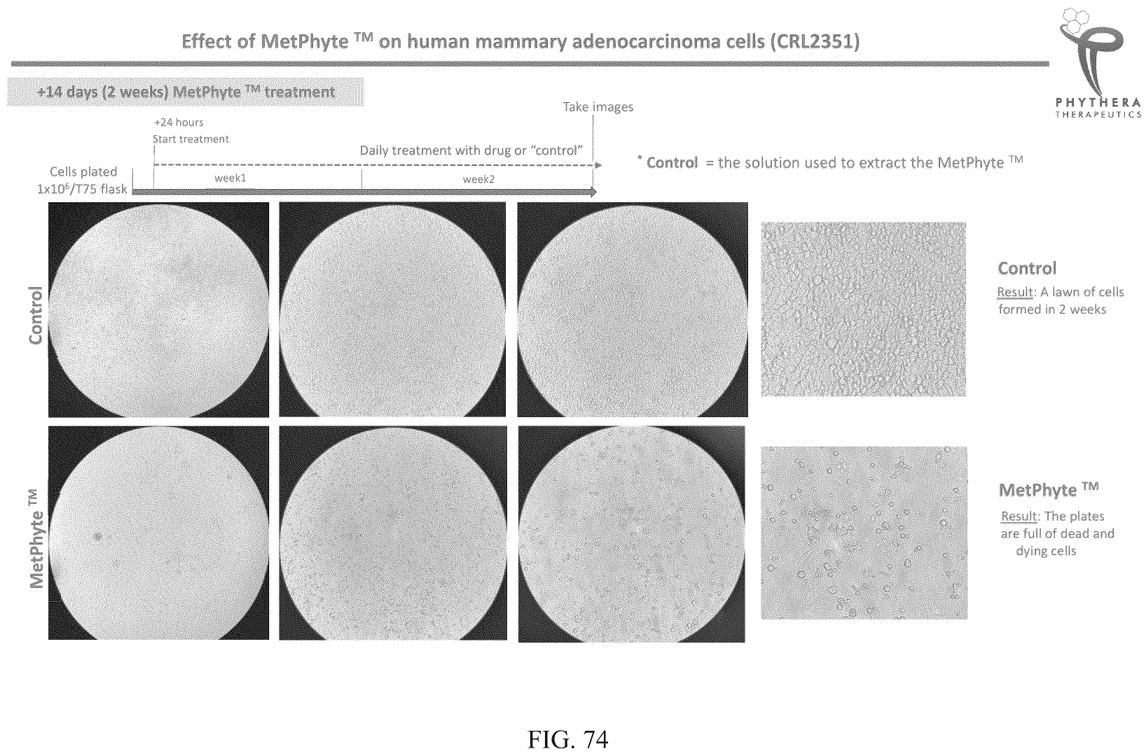

FIG. 74 shows the progress of one experiment for testing one embodiment of MetPhyte on human mammary adenocarcinoma cells CRL2351.

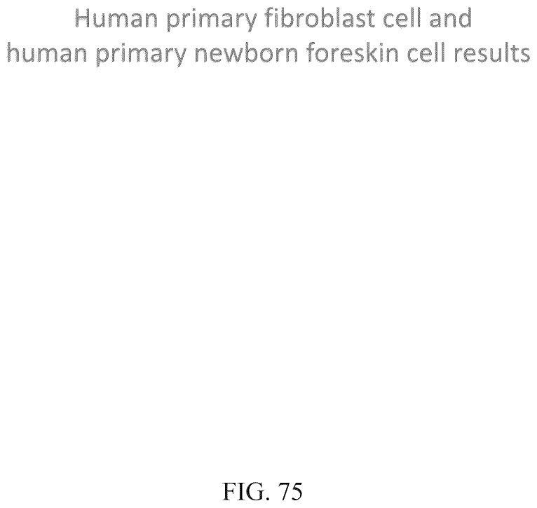

FIG. 75 is a title page for subsequent figures showing efficacy of MetPhyte on human primary fibroblast cell and human primary newborn foreskin cell results.

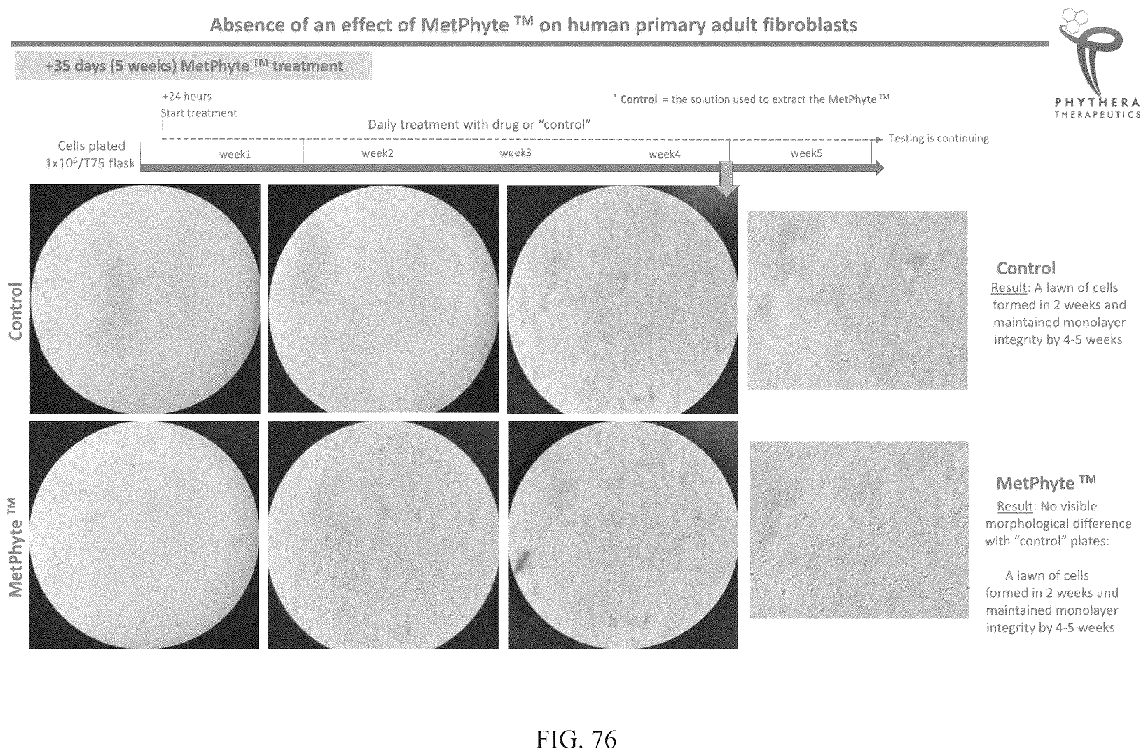

FIG. 76 shows the progress of one experiment for testing one embodiment of MetPhyte on human primary adult fibroblasts.

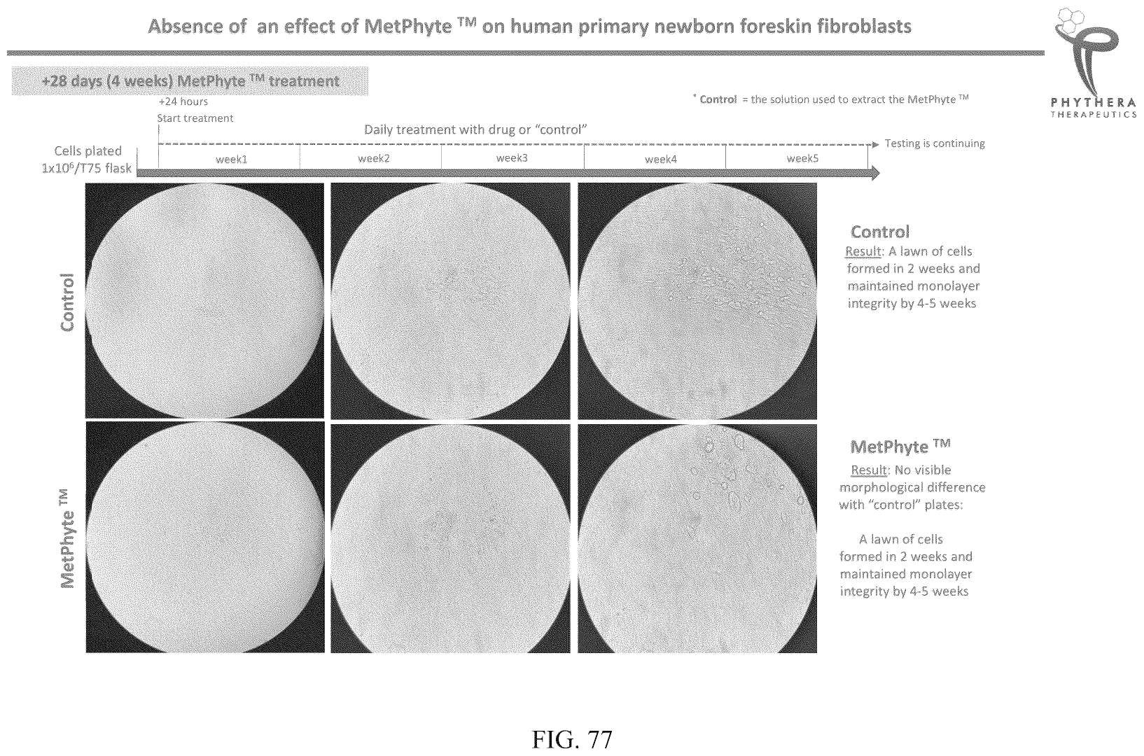

FIG. 77 shows the progress of one experiment for testing one embodiment of MetPhyte on human primary newborn foreskin fibroblasts.

FIG. 78 is a chart identifying a series of gene related cancers and conditions and potential pathways through which MetPhyte may have an effect.

### Alternative Uses

Cell replacement, or any type of cell therapy (to control over-proliferation of cells, a very important problem in cell therapy treatments); ophthalmology, for controlling wet Age-related macular degeneration (Causing blindness) by suppressing over-proliferation of vasculature, causing separation of RPE (retinal pigment epithelium) and photoreceptors (which eventually causes photoreceptor cell death and blindness as a result); ophthalmology, by controlling over-proliferation of epiretinal/subretinal membrane cells (any, but mostly retinal glia)(proliferating vitreoretinopathy) https://eyewiki.aao.org/Proliferative_Vitreoretinopathy; Ophthalmology—Retinal pigment epithelium proliferation (to suppress this process) https://www.ncbi.nlm.nih.gov/pmc/articles/PMC6882585/; Diabetic foot—to reduce cell proliferation (any-vasculature, keratinocytes, fibroblasts—any cells), immune-modulate the pathology site and save feet from amputation https://www.frontiersin.org/articles/10.3389/fendo.2021.744868/full; For immunomodulation in the eye (local, e.g., intra-ocular, to reduce xehogeneic graft rejection, or extra-ocular, to reduce rejection after cornea transplantation, or modulate any ocular pathology caused by immune problems, https://www.ncbi.nlm.nih.gov/pmc/articles/PMC4886870/; Or Immunomodulation anywhere in the body, including for reducing graft-host rejection, any other diseases caused by over-activation of immune system (e.g., but not limited to, auto-immune diseases, for example but not limited to atopic dermatitis/eczema, Pityriasis rosea/rosacea, psoriasis, colitis etc. including but not limited to, when immune cells over-proliferate/became over-active, causing pathology; Or/and Immunomodulation, to improve the outcome of infectious diseases. To summarize 1-6: MetPhyte and MetPhyte PLUS treatment may be productive for modulating the outcomes/treating any diseases, caused by unwanted cell proliferation and unwanted proliferation of any infectious agents. https://courses.lumenlearning.com/suny-ap2/chapter/diseases-associated-with-depressed-or-overactive-immune-responses/; https://www.urmc.rochester.edu/encyclopedia/content.aspx (over-active immune system—any).

Unless otherwise stated, all measurements, values, ratings, positions, magnitudes, sizes, locations, and other specifications that are set forth in this specification, including in the claims that follow, are approximate, not exact. They are intended to have a reasonable range that is consistent with the functions to which they relate and with what is customary in the art to which they pertain.

The foregoing description of the preferred embodiment has been presented for the purposes of illustration and description. While multiple embodiments are disclosed, still other embodiments will become apparent to those skilled in the art from the above detailed description. These embodiments are capable of modifications in various obvious aspects, all without departing from the spirit and scope of protection. Accordingly, the detailed description is to be regarded as illustrative in nature and not restrictive. Also, although not explicitly recited, one or more embodiments may be practiced in combination or conjunction with one another. Furthermore, the reference or non-reference to a particular embodiment shall not be interpreted to limit the scope of protection. It is intended that the scope of protection not be limited by this detailed description, but by the claims and the equivalents to the claims that are appended hereto.

Except as stated immediately above, nothing that has been stated or illustrated is intended or should be interpreted to cause a dedication of any component, step, feature, object, benefit, advantage, or equivalent, to the public, regardless of whether it is or is not recited in the claims.

What is claimed is:

1. A composition comprising:
a botanical substance; and
a modulator.

2. The composition of claim 1, wherein said botanical substance has antitumor properties.

3. The composition of claim 1, wherein said botanical substance is a Chelidonium majus extract.

4. The composition of claim 3, wherein said modulator is tariquidar.

5. The composition of claim 3, wherein said modulator is a transporter enhancer, such that said modulator causes an increase in uptake into one or more cells of said Chelidonium majus extract.

6. The composition of claim 3, wherein said modulator is a transporter inhibitor, such that said modulator causes a decrease in outflow from one or more cells of said Chelidonium majus extract.

7. The composition of claim 4, wherein said Chelidonium majus extract and tariquidar are molecularly linked to generate a linked structure.

8. A method of treating a condition comprising the step of:
administering a composition;
wherein said composition comprises a Chelidonium majus extract and a tariquidar component; and
wherein said condition is a cancer.

9. The method of treating a condition of claim 8, wherein said cancer is selected from the group of cancers consisting of: breast cancer; prostate cancer; lung cancer;
gastrointestinal cancer; and skin cancer.

10. The method of treating a condition of claim 8, wherein said condition is related to a gene selected from the group of genes consisting of: STAG1A; STAG1B; FAM72C; ATF7-NPFF; SLX1B; MTRNR2L10; TMSB15A; Tb15; TbNB; TMSL8; TMSNB; TMSB15; TMSB15B; GP2; CSAG3; DNAJC25-GNG10; PIF1; BIVM; ERCC5; CSNK2A3; CT47A10; DRD2; BRINP2; PNPLA3; KLK13; and SERPINA6.

11. The method of treating a condition of claim 8, wherein said administration of said composition is by application to a user's skin.

12. The method of treating a condition of claim 8, wherein said administration of said composition is conducted intravenously.

13. The method of treating a condition of claim 8, wherein said administration of said composition is conducted orally.

14. The method of treating a condition of claim 8, wherein said administration of said composition is conducted by introducing said composition directly to a tumor.

15. The method of treating a condition of claim 8, wherein preferably 0.1-10 g in dry weight of said Chelidonium majus extract and 2-4 mg of said tariquidar component per kg of a user of said composition is administered per day.

16. A method of treating a condition comprising the steps of:
administering to a user a Chelidonium majus extract; and
administering to said user a modulator.

17. The method of treating a condition of claim 16, wherein said modulator is tariquidar.
